SEQUENCE LISTING (211 sequences)

SEQ ID NO: 1 - DNA, 30 bp, Artificial Sequence
  KPC_forward
cccatctcggaaaaatatctgacaacaggc

SEQ ID NO: 2 - DNA, 35 bp, Artificial Sequence
  KPC_reverse
ctccagctcccagcggtccagacggaacgtggtat

SEQ ID NO: 3 - DNA, 48 bp, Artificial Sequence
  KPC_probe
cgtgcaatacagtgataacgccgccgccaatttgttgctgaaggagtt

SEQ ID NO: 4 - DNA, 30 bp, Artificial Sequence
  OXA-48-like_forward
ccttatcggctgtgtttttggtggcatcga

SEQ ID NO: 5 - DNA, 31 bp, Artificial Sequence
  OXA-48-like_reverse
cccgtttaagattattggtaaatccttgctg

SEQ ID NO: 6 - DNA, 48 bp, Artificial Sequence
  OXA-48-like_probe
tggcaagaaaacaaaagttggaatgctcactttactgaacataaatca

SEQ ID NO: 7 - DNA, 35 bp, Artificial Sequence
  NDM_forward
gaccagaccgcccagatcctcaactggatcaagca

SEQ ID NO: 8 - DNA, 32 bp, Artificial Sequence
  NDM_reverse
ctggttcgacaacgcattggcataagtcgcaa

SEQ ID NO: 9 - DNA, 49 bp, Artificial Sequence
  NDM_probe
ccccgccgcatgcagcgcgtccataccgcccatcttgtcctgatgcgcg

SEQ ID NO: 10 - DNA, 29 bp, Artificial Sequence
  IMP_forward_1
ggaatagagtggcttaattctcaatctat

SEQ ID NO: 11 - DNA, 29 bp, Artificial Sequence
  IMP_forward_2
ggaatagagtggcttaattctcgatctat

SEQ ID NO: 12 - DNA, 30 bp, Artificial Sequence
  IMP_reverse
aaaaaacttcaattttatttttaactagcc

SEQ ID NO: 13 - DNA, 30 bp, Artificial Sequence
  IMP_reverse_2
aaaaaatttcaatctttttcttaactaacc

SEQ ID NO: 14 - DNA, 30 bp, Artificial Sequence
  IMP_reverse_3
aaaaaacttcaattttgtttttaattagtg

SEQ ID NO: 15 - DNA, 30 bp, Artificial Sequence
  IMP_reverse_4
aaaaaatttcaattttatttttaagtagcc

SEQ ID NO: 16 - DNA, 30 bp, Artificial Sequence
  IMP_reverse_5
agaaaacttcaattttgtttttgataagtg

SEQ ID NO: 17 - DNA, 30 bp, Artificial Sequence
  IMP_reverse_6
aaaacacttctattttatctttcgccagcc

SEQ ID NO: 18 - DNA, 30 bp, Artificial Sequence
  IMP_reverse_7
aaaaaacttcaattttgttttttattagtg

SEQ ID NO: 19 - DNA, 27 bp, Artificial Sequence
  IMP_reverse_8
ttcaattttatttttaactagccaata

SEQ ID NO: 20 - DNA, 27 bp, Artificial Sequence
  IMP_reverse_9
ttcaatctttttcttaactaaccaata

SEQ ID NO: 21 - DNA, 27 bp, Artificial Sequence
  IMP_reverse_10
ttcaattttgtttttaattagtgaata

SEQ ID NO: 22 - DNA, 27 bp, Artificial Sequence
  IMP_reverse_11
ttctattttatctttcgccagccaata

SEQ ID NO: 23 - DNA, 50 bp, Artificial Sequence
  IMP_probe
cccacgtatgcatctgaattaacaaatgaactgcttaaaaaagacggtaa

SEQ ID NO: 24 - DNA, 33 bp, Artificial Sequence
  VIM_forward
ccagattgccgatggtgtttggtcgcatatcgc

SEQ ID NO: 25 - DNA, 35 bp, Artificial Sequence
  VIM_reverse
ggaagtccaatttgcttttcaatctccgcgagaag

SEQ ID NO: 26 - DNA, 47 bp, Artificial Sequence
  VIM_probe
ctcattgtccgtgatggtgatgagttgcttttgattgatacagcgtg

SEQ ID NO: 27 - DNA, 48 bp, Artificial Sequence
  VIM_probe_2
cttatcgtccgcgatgctgatgagttgcttcttattgatacagcgtgg

SEQ ID NO: 28 - DNA, 29 bp, Artificial Sequence
  IMI_forward
cttgaacgatatatcggtggtcctgaggg

SEQ ID NO: 29 - DNA, 31 bp, Artificial Sequence
  IMI_reverse
ttcatcgccaggaatagctgtgtttagatct

SEQ ID NO: 30 - DNA, 29 bp, Artificial Sequence
  IMI_reverse_2
gtgtcacgttcatcgccaggaatagctgt

SEQ ID NO: 31 - DNA, 49 bp, Artificial Sequence
  IMI_probe
ctaaattcatgcggtcgattggagataaagattttagactcgatcgttg

SEQ ID NO: 32 - DNA, 35 bp, Artificial Sequence
  CTX-M-15_forward
ccgaatctgttaaatcagcgagttgagatcaaaaa

SEQ ID NO: 33 - DNA, 35 bp, Artificial Sequence
  CTX-M-14_forward
cacgtcaacggcacaatgacgctggcagaactgag

SEQ ID NO: 34 - DNA, 35 bp, Artificial Sequence
  CTX-M-15_reverse
ctagccgggccgccaacgtgagcaatcagcttatt

SEQ ID NO: 35 - DNA, 35 bp, Artificial Sequence
  CTX-M-14_reverse
tccagacgaaacgtctcatcgccgatcgcgcgggc

SEQ ID NO: 36 - DNA, 47 bp, Artificial Sequence
  CTX-M-15_probe
gattgcggaaaagcacgtcaatgggacgatgtcactggctgagctta

SEQ ID NO: 37 - DNA, 50 bp, Artificial Sequence
  CTX-M-14_probe
agtacagcgacaataccgccatgaacaaattgattgcccagctcggtggc

SEQ ID NO: 38 - DNA, 798 bp, Shewanella sp.
atgcgtgcgttagccttatcggctgtgttgctggtgacaacgatgattggcatgcctgcggtggcaaaggagtggcaagagaacaagagctggaatgctcactttagcgaacataaaacccaaggcgtggttgtgctgtggaacgagaatacacagcagggttttactaacgatcttaaacgggcaaaccaagcatttttacctgcatcgacctttaagatcccaaacagtttaattgccttggacttaggtgtggttaaggatgagcatcaagtctttaaatgggatggacagacgcgagatatcgccgcatggaatcgcgaccatgatttaatcaccgcgatgaagtattcggttgtgcctgtttatcaagaatttgcccgccaaattggcgaggctcgtatgagtaaaatgctgcacgccttcgattatggcaatgaggatatctcgggcaatttagacagcttttggctcgatggtggtattcgaatttcggctacccagcaaatcgcttttttacgcaagctgtaccacaacaagctgcacgtttctgagcgtagtcagcgcatcgtcaagcaagccatgctgaccgaggcaaatgccgattatattatccgagcgaaaactggttactcgacgcgaattgagccgaaaattggttggtgggttggctgggtcgaactcgatgacaatgtgtggttcttcgcgaccaatatggatatgccttccgctgagggcctagggttacgtcaaagcattacgaaaacagtgctgaaacaggaaaaaattattccttag

SEQ ID NO: 39 - DNA, 798 bp, Shewanella sp.
atgcgtgcgttagccttatcggctgtgttgatggtgacaacgatgattggcatgcctgcggtggcaaaggagtggcaagagaacaagagctggaatgctcactttagcgaacataaaacccaaggcgtggttgtgctctggaacgagaatacacagcagggttttactaacgatcttaaacgggcaaaccaagcatttttaccggcatcgacctttaagatcccaaacagtttaattgccttggacttaggtgtggttaaggatgagcatcaggtctttaaatgggatggacagacgcgagatatcgccgcatggaatcgtgaccatgatttaatcaccgcgatgaagtattcggttgtgcctgtttatcaagaatttgcccgccaaattggcgaggctcgtatgaataaaatgctgcacgccttcgattatggcaatgaggatatctcgggcaatttggacagtttttggctcgatggtggtattcgcatttcggctacccagcaaatcgcttttttacgcaagctgtaccacaacaagctgcacgtttctgagcgtagtcagcgcatcgtgaaacaagccatgctgaccgaggcaaatgccgactatatcatccgagcgaaaaccggctattcggtcagaattgaaccgaaaatcggttggtgggttggctgggtcgaactggatgataatgtgtggtttttcgcgaccaatatggatatgccttccgctgagggcttagggttacgtcaaagcattacgaaagcagtgctgaaacaggaaaaaattattccttag

SEQ ID NO: 40 - DNA, 798 bp, Shewanella sp.
atgcgtgcgttagccttatcggctgtgttgatggtgacaacgatgattggcatgcctgcggtggcaaaggagtggcaagagaacaagagttggaatgctcactttagcgaacataaaacccaaggcgtggttgtgctctggaacgagaataaacagcagggttttactaacgatcttaaacgggcaaaccaagcatttttaccggcatcgacttttaagatcccaaacagtttaattgccttggacttaggtgtggttaaggatgagcatcaggtctttaaatgggatggacagacgcgagatatcgccgcgtggaatcgtgaccatgacttaattaccgcgatgaagtactcggttgtgcctgtttatcaagaatttgcccgccaaattggcgaggcgcgtatgagtaaaatgctgcatgccttcgattatggcaatgaggatatctcgggcaatttggacagtttttggctcgatggtggtattcgcatttcggctacccagcaaatcgcttttttacgcaagctctaccacaacaagctgcacgtctctgagcgtagtcagcgcatcgtcaaacaagccatgctgaccgaagcaaatgccgactatatcattcgggcgaaaacgggttattcggtcagaattgagccgaaaatcggttggtgggttggctgggtcgaactcgatgacaatgtgtggttcttcgcgaccaatatggatatgccttccgctgagggcttagggttacgtcaaagcattacgaaagcagtgctaaaacaggaaaaaattattccttag

SEQ ID NO: 41 - DNA, 798 bp, Citrobacter freundii
atgcgtgtattagccttatcggctgtgtttttggtggcatcgattatcggaatgccagcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgtgatatcgccgcttggaatcgtgaccatgacttaattaccgcgatgaagtactcagttgtgcctgtttatcaagaatttgcccgccaaattggtgaggcacgtatgagtaaaatgctgcacgccttcgattatggcaatgaggatatctcgggcaatgtagacagtttttggctcgatggtggtattcgcatttcggctacccagcaaatcgcttttttacgcaagctgtatcacaacaagctgcacgtttctgagcgtagtcagcgcatcgtgaaacaagccatgctgaccgaagccaatggcgactatattattcgggctaaaacgggatactcgactagaatcgaacctaagattggctggtgggttggttgggttgaacttgatgataatgtgtggttttttgcgatgaatatggatatgcccacatcggatggtttagggctgcgccaagccatcacaaaagaagtgctcaaacaggagaaaattattccctag

SEQ ID NO: 42 - DNA, 798 bp, Klebsiella pneumoniae; identical to SEQ ID NO: 41

SEQ ID NO: 43 - DNA, 798 bp, Enterobacter cloacae; identical to SEQ ID NO: 41

SEQ ID NO: 44 - DNA, 798 bp, Escherichia coli
atgcgtgtattagccttatcggctgtgtttttggtggcatcgattatcggaatgccagcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgtgatatcgccgcttggaatcgtgaccatgacttaattaccgcgatgaagtactcagttgtgcctgtttatcaagaatttgcccgccaaattggtgaggcacgtatgagtaaaatgctgcacgccttcgattatggcaatgaggatatctcgggcaatgtagacagtttttggctcgatggtggtattcgcatttcggctacccagcaaatcgcttttttacgcaagctgtatcacaacaagctgcacgtttctgagcgtagtcagcgcatcgtgaaacaagccatgctgaccgaagccaatggcgactatattattcgggctaaaacgggatactcgactagtatcgaacctaagattggctggtgggttggttgggttgaacttgatgataatgtgtggttttttgcgatgaatatggatatgcccacatcggatggtttagggctgcgccaagccatcacaaaagaagtgctcaaacaggagaaaattattccctag

SEQ ID NO: 45 - DNA, 786 bp, Klebsiella pneumoniae
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggcgactatattattcgggctaaaactggatccaatactaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 46 - DNA, 786 bp, Enterobacter cloacae
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggcgactatattattcgggctaaaactggatacgatactaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 47 - DNA, 798 bp, Shewanella xiamenensis
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgatggtggtattcgaatttcggccactgagcaaatcagctttttaagaaagctgtatcacaataagttacacgtatcggagcgcagtcagcgcatcgtgaaacaagccatgctgaccgaagccaatggcgactatattattcgggctaaaacgggatactcgactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 48 - DNA, 798 bp, Shewanella xiamenensis
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacatcggcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgatggtggtattcgaatttcggccactgagcaaatcagctttttaagaaagctgtatcacaataagttacacgtatcggagcgcagccagcgtattgtcaaacaagccatgctgaccgaagccaatggcgactatattattcgggctaaaacgggatactcgactagaatcgaacctaagattggctggtgggtcggttgggttgaactggatgataatgtgtggttttttgcgatgaatatggatatgcccacatcggatggtttagggctgcgccaagccatcacaaaagaagtgctcaaacaggaaaaaattattccctag

SEQ ID NO: 49 - DNA, 798 bp, Klebsiella pneumoniae; identical to SEQ ID NO: 48

SEQ ID NO: 50 - DNA, 798 bp, Shewanella xiamenensis
atgcgtgtattagccttatcggctgtgtttttggtggcatcgattatcggaatgcctgcggtagcaaaggaatggcaagaaaacaaaagttggaatgctcactttactgaatataaatcacagggcgtagttgcgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtaggcagtttctggctcgacggtggtattcgaatttcggccactgagcaaatcagctttttaagaaagctgtatcacaataagttacacgtatcggagcgcagccagcgtattgtcaaacaagccatgctgaccgaagccaatggcgactatattattcgggctaaaactggatactcgactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 51 - DNA, 798 bp, Enterobacter spp.
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggcgactatattattcgggctaaaactggatacgagactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 52 - DNA, 798 bp, Klebsiella pneumoniae
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaatactttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 53 - DNA, 798 bp, Klebsiella pneumoniae
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcggctagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 54 - DNA, 798 bp, Klebsiella pneumoniae; identical to SEQ ID NO: 53

SEQ ID NO: 55 - DNA, 798 bp, Klebsiella pneumoniae
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 56 - DNA, 798 bp, Enterobacter cloacae; identical to SEQ ID NO: 55

SEQ ID NO: 57 - DNA, 798 bp, Escherichia coli; identical to SEQ ID NO: 55

SEQ ID NO: 58 - DNA, 798 bp, Enterobacter cloacae; identical to SEQ ID NO: 55

SEQ ID NO: 59 - DNA, 798 bp, Klebsiella pneumoniae; identical to SEQ ID NO: 55

SEQ ID NO: 60 - DNA, 798 bp, Klebsiella pneumoniae; identical to SEQ ID NO: 55

SEQ ID NO: 61 - DNA, 798 bp, Klebsiella pneumoniae; identical to SEQ ID NO: 55

SEQ ID NO: 62 - DNA, 798 bp, Klebsiella pneumoniae; identical to SEQ ID NO: 55

SEQ ID NO: 63 - DNA, 798 bp, Klebsiella pneumoniae; identical to SEQ ID NO: 55

SEQ ID NO: 64 - DNA, 798 bp, Klebsiella pneumoniae
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactggaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 65 - DNA, 798 bp, Pseudomonas aeruginosa
atgtttcaaattcgcagctttctggttggtatcagtgcattcgtcatggccgtacttggatcagcagcatattccgcacagcctggcggtgaatatccgacagtagatgacataccggtaggggaagttcggctgtacaagattggcgatggcgtttggtcgcatatcgcaactcagaaactcggtgacacggtgtactcgtctaatggacttatcgtccgcgatgctgatgagttgcttcttattgatacagcgtggggggcgaagaacacggtagcccttctcgcggagattgaaaagcaaattggacttccagtaacgcgctcaatttctacgcacttccatgacgatcgagtcggtggagttgatgtcctccgggcggctggagtggcaacgtacacctcacccttgacacgccagctggccgaagcggcgggaaacgaggtgcctgcgcactctctaaaagcgctctcctctagtggagatgtggtgcgcttcggtcccgtagaggttttctatcctggtgctgcgcattcgggcgacaatcttgtggtatacgtgccggccgtgcgcgtactgtttggtggctgtgcagttcatgaggcgtcacgcgaatccgcgggtaatgttgccgatgccaatttggcagaatggcctgctaccattaaacgaattcaacagcggtatccggaagcagaggtcgtcatccccggccacggtctaccgggcggtctggaattgctccaacacacaactaacgttgtcaaaacgcacaaagtacgcccggtggccgagtaa

SEQ ID NO: 66 - DNA, 801 bp, Pseudomonas aeruginosa
atgttaaaagttattagtagtttattgttctacatgaccgcctctctaatggctgtagctagtccgttagcccattccggggagtcgagaggtgagtatccgacagtcagcgaaattccggtcggagaagttcggctgtaccagattgacgatggtgtttggtcgcatatcgcaacgcatacgtttgatggcgtggtgtacccgtccaatggtctcattgtccgtgatggcgatgagttgcttttgattgatacagcttggggtacgaaaaacacagtggcccttctcgcggagattgagaagcaaattggacttcccgtaacgcgttcagtctccacgcactttcatgacgaccgcgtcggcggagttgatgcccttagggcggctggagtggcgacgtacgcatcgccctcgacacgccgtctagccgaggcagaggggaacgaggttcccacacactctctagaagggctctcatcgagtggggacgcagtgcgtttcggtccagtagagctcttctatcctggtgctgcgcattcgaccgacaatctggttgtatacgtcccgtcagcgaacgtgctatacggtggttgtgccgttctggaattgtcacgcacatccgcgggaaacgtggccgatgccgacctggctgaatggcccggttccgttgagcggattcaacaacattacccagaagcagaggtggtcattcccgggcacggtctaccgggcggtctagacttgctccagcacacagcgaacgttgtcaaagcacacacaaatcgctcagtcgccgagtag

SEQ ID NO: 67 - DNA, 789 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcggtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagcgaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 68 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcggcacgccggctagccgaggtagaggggaacgagattcccacgcactctctagaaggactctcatcgagcggggacgcggtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccagaagcacagttcgtcattccggggcacggcctgccgggcggtctagacttgctcaagcacacaacgaatgttgtaaaagcgcacacaaatcgctcagtcgttgagtag

SEQ ID NO: 69 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgaagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggagcgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 70 - DNA, 801 bp, Pseudomonas aeruginosa; identical to SEQ ID NO: 69

SEQ ID NO: 71 - DNA, 801 bp, Acinetobacter baumannii
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggagcgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 72 - DNA, 801 bp, Pseudomonas aeruginosa; identical to SEQ ID NO: 71

SEQ ID NO: 73 - DNA, 801 bp, Acinetobacter haemolyticus; identical to SEQ ID NO: 71

SEQ ID NO: 74 - DNA, 801 bp, Enterobacter cloacae
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgattcatgagttgtcacgcacgtctgcggggaacgtggccgatgccgatctggctgaatggcccacctccattgagcggattcaacaacgctacccggaagcacagttcgtcattccggggcacggcctgccgggcggtctagacttgctcaagcacacaacgaatgttgtaaaagcgcacacaaatcgctcagtcgttgagtag

SEQ ID NO: 75 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacgctacccggaagcacagttcgtcattccggggcacggcctgccgggcggtctagacttgctcaagcacacaacgaatgttgtaaaagcgcacacaaatcgctcagtcgttgagtag

SEQ ID NO: 76 - DNA, 801 bp, Pseudomonas aeruginosa
atgttcaaacttttgagtaagttattggtctatttgaccgcgtctatgatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 77 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctttggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 78 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggttgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 79 - DNA, 801 bp, Enterobacter cloacae
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcaagcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 80 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgatacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 81 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagtacgtcattccggggcacggcctgccgggcggtctagacttgctcaagcacacaacgaatgttgtaaaagcgcacacaaatcgctcagtcgttgagtag

SEQ ID NO: 82 - DNA, 801 bp, Pseudomonas putida
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcggtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggagcgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 83 - DNA, 801 bp, Pseudomonas putida; identical to SEQ ID NO: 82

SEQ ID NO: 84 - DNA, 801 bp, Klebsiella pneumoniae
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcactcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 85 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcggtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 86 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcaacgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 87 - DNA, 801 bp, Pseudomonas aeruginosa
atgttcaaacttttgagtaagttattggtctatttgaccgcgtctatcatggctattgcgagtccgctcgctttttccgtagattctagcggtgagtatccgacagtcagcgaaattccggtcggggaggtccggctttaccagattgccgatggtgtttggtcgcatatcgcaacgcagtcgtttgatggcgcagtctacccgtccaatggtctcattgtccgtgatggtgatgagttgcttttgattgatacagcgtggggtgcgaaaaacacagcggcacttctcgcggagattgagaagcaaattggacttcctgtaacgcgtgcagtctccacgcactttcatgacgaccgcgtcggcggcgttgatgtccttcgggcggctggggtggcaacgtacgcatcaccgtcgacacgccggctagccgaggtagaggggaacgagattcccacgcactctctagaaggactctcatcgagcggggacgcagtgcgcttcggtccagtagaactcttctatcctggtgctgcgcattcgaccgacaacttagttgtgtacgtcccgtctgcgagtgtgctctatggtggttgtgcgatttatgagttgtcacgcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 88 - DNA, 801 bp, Pseudomonas aeruginosa; identical to SEQ ID NO: 87

SEQ ID NO: 89 - DNA, 801 bp, Pseudomonas aeruginosa
atgttaaaagttattagtagtttattggtctatttgaccgcgtctgtcatggctgtagctagtccgttagcccattccggggagccgagtggtgagtatccgacagtcaacgaaattccggtcggagaggtccggctttaccagattgctgatggtgtttggtcgcatatcgcaacgcagtcgtttgatggcgcggtctacccatccaatggtctcattgtccgtgatggtgatgagttgcttttgattgatacagcgtggggtgcgaaaaacacagcggcccttctcgcggagattgagaagcaaattggacttcccgtaacgcgtgcagtctccacgcactttcatgacgaccgcgtcggcggcgttgatgtccttcggaaggctggagtggcaacgtacgcatcaccgtcgacacgccggctagccgaggcagaggggaacgagattcccacgcactctctagaaggactctcatcgagcggggacgcagtgcgcttcggtccagtagagctcttctatcccggtgctgcgcattcgaccgacaatctggttgtatacgtcccgtcagcgaacgtgctatacggtggttgtgccgttcttgcgttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcacagcacacaaaaatcgctcagtcgttgagtag

SEQ ID NO: 90 - DNA, 801 bp, Klebsiella pneumoniae
atgttaaaagttattagtagtttattggtctacatgaccgcgtctgtcatggctgtagctagtccgttagcccattccggggagccgagtggtgagtatccgacagtcaacgaaattccggtcggagaggtccggctttaccagattgctgatggtgtttggtcgcatatcgcaacgcagtcgtttgatggcgcggtctacccatccaatggtctcattgtccgtgatggtgatgagttgcttttgattgatacagcgtggggtgcgaaaaacacagcggcccttctcgcggagattgagaagcaaattggacttcccgtaacgcgtgcagtctccacgcactttcatgacgaccgcgtcggcggcgttgatgtccttcggaaggctggagtggcaacgtacgcatcaccgtcgacacgccggctagccgaggcagaggggaacgagattcccacgcactctctagaaggactctcatcgagcggggacgcagtgcgcttcggtccagtagagctcttctatcccggtgctgcgcattcgaccgacaatctggttgtatacgtcccgtcagcgaacgtgctatacggtggttgtgccgttcttgcgttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcacagcacacaaaaatcgctcagtcgccgagtag

SEQ ID NO: 91 - DNA, 801 bp, Klebsiella pneumoniae
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcaagcacgtctgcggggaacgtggccgatgccgatctggctgaatggcccacctccattgagcggattcaacaacactacccggaagcacagttcgtcattccggggcacggcctgccgggcggtctagacttgctcaagcacacaacgaatgttgtaaaagcgcacacaaatcgctcagtcgttgagtag

SEQ ID NO: 92 - DNA, 801 bp, Klebsiella pneumoniae
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcaatctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcaagcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 93 - DNA, 801 bp, Pseudomonas aeruginosa
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatctcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 94 - DNA, 801 bp, Klebsiella pneumoniae
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatctcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcaagcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 95 - DNA, 801 bp, Klebsiella pneumoniae; identical to SEQ ID NO: 94

SEQ ID NO: 96 - DNA, 801 bp, Klebsiella pneumoniae
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcttgagttgtcaagcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 97 - DNA, 801 bp, Pseudomonas aeruginosa
atgttaaaagttattagtagtttattggtctacatgaccgcgtctgtcatggctgtcgcaagtccgttagcccattccggggagccgagtagtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 98 - DNA, 801 bp, Pseudomonas aeruginosa; identical to SEQ ID NO: 97

SEQ ID NO: 99 - DNA, 801 bp, Escherichia coli
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaaagtgctatacggtggttgtgccgttcatgagttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 100 - DNA, 801 bp, Escherichia coli; identical to SEQ ID NO: 99

SEQ ID NO: 101 - DNA, 801 bp, Pseudomonas aeruginosa
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcttgagttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 102 - DNA, 801 bp, Aeromonas hydrophila
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcacgcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 103 - DNA, 801 bp, Pseudomonas aeruginosa; identical to SEQ ID NO: 102

SEQ ID NO: 104 - DNA, 801 bp, Pseudomonas aeruginosa; identical to SEQ ID NO: 102

SEQ ID NO: 105 - DNA, 801 bp, Escherichia coli
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaaagtgctatacggtggttgtgccgttcatgagttgtcaagcacgtctgcggggaacgtggccgatgccgatctggctgaatggcccacctccgttgagcggattcaaaaacgctacccggaagcagaggtcgtcattcccgggcacggtctaccgggcggtctagacttgctccagcacacagcgaacgttgtcaaagcacacaaaaatcgctcagtcgccgagtag

SEQ ID NO: 106 - DNA, 800 bp, Klebsiella pneumoniae
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctattcggtggttgtgccgttcatgagttgtcaagcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagta

SEQ ID NO: 107 - DNA, 801 bp, Klebsiella oxytoca
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcaagcacgtctgcggggaacgtgaccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 108 - DNA, 801 bp, Klebsiella oxytoca
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagcggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcaagcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 109 - DNA, 801 bp, Klebsiella pneumoniae
atgttaaaagttattagtagtttattggtctacatgaccgcgtctgtcatggctgtcgcaagtccgttagcccattccggggagccgagtggtgagtatccgacagtcaacgaaattccggtcggagaggtccgactttaccagattgccgatggtgtttggtcgcatatcgcaacgcagtcgtttgatggcgcggtctacccgtccaatggtctcattgtccgtgatggtgatgagttgcttttgattgatacagcgtggggtgcgaaaaacacagcggcacttctcgcggagattgaaaagcaaattggacttcccgtaacgcgtgcagtctccacgcactttcatgacgaccgcgtcggcggcgttgatgtccttcgggcggctggggtggcaacgtacgcatcaccgtcgacacgccggctagccgaggcagaggggaacgagattcccacgcattctctagaaggactctcatcgagcggggacgcagtgcgcttcggtccagtagagctcttctatcctggtgctgcgcattcgaccgacaatctggttgtatacgtcccgtcagcgaacgtgctatacggtggttgtgccgttcatgagttgtcaagcacgtctgcggggaacgtggccgatgccgatctggctgaatggcccacctccgttgagcggattcaaaaacactacccggaagcagaggtcgtcattcccgggcacggtctaccgggcggtctagacttgctccagcacacagcgaacgttgtcaaagcacacaaaaatcgctcagtcgccgagtag

SEQ ID NO: 110 - DNA, 801 bp, Pseudomonas aeruginosa; identical to SEQ ID NO: 109

SEQ ID NO: 111 - DNA, 801 bp, Proteus mirabilis
atgttaaaagttattagtagtttattggtctacatgaccgcgtctgtcatggctgtagctagtccgttagcccattccggggagccgagtggtgagtatccgacagtcaacgaaattccggtcggagaggtccggctttaccagattgctgatggtgtttggtcgcatatcgcaacgcagtcgtttgatggcgcggtctacccatccaatggtctcattgtccgtgatggtgatgagttgcttttgattgatacagcgtggggtgcgaaaaacacagcggcccttctcgcggagattgagaagcaaattggacttcccgtaacgcgtgcagtctccacgcactttcatgacgaccgcgtcggcggcgttgatgtccttcggaaggctggagtggcaacgtacgcatcaccgtcgacacgccggctagccgaggcagaggggaacgagattcccacgcactctctagaaggactctcatcgagcggggacgcagtgcgcttcggtccagtagagctcttctatcccggtgctgcgcattcgaccgacaatctggttgtatacgtcccgtcagcgaacgtgctatacggtggttgtgccgttcttgcgttgtcacgcacgtctgcggggaacgtggccgatgccgatctggctgaatggcccacctccgttgagcggattcaaaaacactacccggaagcacagttcgtcattccggggcacggcctgccgggcggtctagacttgctcaagcacacaacgaatgttgtaaaagcgcacacaaatcgctcagtcgttgagtag

SEQ ID NO: 112 - DNA, 801 bp, Enterobacter cloacae; identical to SEQ ID NO: 90

SEQ ID NO: 113 - DNA, 801 bp, Klebsiella pneumoniae; identical to SEQ ID NO: 99

SEQ ID NO: 114 - DNA, 801 bp, Pseudomonas aeruginosa; identical to SEQ ID NO: 90

SEQ ID NO: 115 - DNA, 849 bp, Klebsiella pneumoniae
cgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgatgatgcc

SEQ ID NO: 116 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttcggtggtcacccatctcggaaaaatatctgacaacaggcatgacggttttggagctgtccgcggccgccgtgcaatacagtgataacgccgccgccaatttgttgctgaaggagttgggcggcccggccaaactgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagggtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggcgtcaacgggcagtaa

SEQ ID NO: 117 - DNA, 876 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatgcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 118 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggttcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgtcgcagcggcagcagcttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 119 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagatgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 120 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaagcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 121 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagcttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 122 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 123 - DNA, 881 bp, Enterobacter sp.
tgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttcggtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagggtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 124 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttctgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 125 - DNA, 882 bp, Pseudomonas aeruginosa
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttcggtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 126 - DNA, 882 bp, Enterobacter cloacae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctgggcacacccatccgttacggcaaaaacgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggactgctgggagctggagctgaactccgctatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagtacagcgaggccgtcatcgccgctgcggctggactcgcgctcgagggattgggcgtcaacgggcagtaa

SEQ ID NO: 127 - DNA, 882 bp, Enterobacter cloacae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctgggcacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgctatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggcgtcaacgggcagtaa

SEQ ID NO: 128 - DNA, 854 bp, Escherichia coli
gccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagcgtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggc

SEQ ID NO: 129 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagggtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaa

SEQ ID NO: 130 - DNA, 882 bp, Acinetobacter baumannii
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttcggtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggcgtcaacgggcagtaa

SEQ ID NO: 131 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagggtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggcgtcaacgggcagtaa

SEQ ID NO: 132 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggcgtcaacgggcagtaa

SEQ ID NO: 133 - DNA, 882 bp, Klebsiella pneumoniae; identical to SEQ ID NO: 132

SEQ ID NO: 134 - DNA, 882 bp, Klebsiella pneumoniae
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatagataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagtacagcgaggccgtcatcgccgctgcggctagactcgcgctcgagggattgggcgtcaacgggcagtaa

SEQ ID NO: 135 - DNA, 813 bp, Klebsiella pneumoniae
atggaattgcccaatattatgcacccggtcgcgaagctgagcaccgcattagccgctgcattgatgctgagcgggtgcatgcccggtgaaatcagcccgacgattgaccagcaaatggaaactggcgaccaacggtttggcgatctggttttccgccagctcgcaccgaatgtctggcagcacacttcctatctcgacatgccgagtttcggggcagtcacttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgaccgcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 136 - DNA, 813 bp, Escherichia coli
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatgggcgcgctgcatgcggcggggattgcgacttatgccaatgcgttgtcgaaccagcttgccccgcaagaggggctggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 137 - DNA, 813 bp, Escherichia coli
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatgaacgcgctgcatgcggcggggattgcgacttatgccaatgcgttgtcgaaccagcttgccccgcaagaggggctggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 138 - DNA, 871 bp, Escherichia coli
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctgttggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggctggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctgagccatggctgaccacgtcacccccaatctgccatcgcgcgatttcgatgtgacagaga

SEQ ID NO: 139 - DNA, 813 bp, Escherichia coli
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggctggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 140 - DNA, 813 bp, Klebsiella pneumoniae
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaaaaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 141 - DNA, 813 bp, Escherichia coli
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccaatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 142 - DNA, 813 bp, Escherichia coli
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagtgcgcgcgtttggtgcggcgttccccaaggccagcatgatcgtgatgagccattccgcccccgatagccgcgccgcaatcactcatacggcccgcatggccgacaagctgcgctga

SEQ ID NO: 143 - DNA, 813 bp, Klebsiella pneumoniae
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 144 - DNA, 813 bp, Acinetobacter baumannii
atggaattgcccaatattatgcacccggtcgcgaagctgagcaccgcattagccgctgcattgatgctgagcgggtgcatggccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 145 - DNA, 741 bp, Pseudomonas aeruginosa
atgaaaaaattatttgttttatgtgtattcttcctttgcaacattgctgctgcagatgattctttgcctgatttaaaaattgagaagcttgaaaaaggcgtttatgttcatacttcgtttgaagaagttaaaggttggggtgtagtcacaaaacacggtttagtggttcttgtaaagaatgatgcttatctgatagatactccaattaccgctaaagatactgaaaaattagttaattggtttattgagcacggctatagaatcaaaggcagtatttccacacatttccatggcgacagtacggctggaatagagtggcttaattctcaatctatctccacgtatgcctctgaattaacaaatgaacttctaaaaaaagacaataaggtgcaagctacaaattcttttagtggagttagttattcacttatcaaaaacaaaattgaagttttctatccaggtccaggacacactcaagataacgtagtggtttggttacctgaaaagaaaattttattcggtggttgctttgttaaaccggacggtcttggaaatttaggggatgcaaatttagaagcttggccaaagtccgctaaaatattaatgtctaaatatggtaaagcaaaactggttgtttcaagtcatagtgaaattggaaacgcatcactcttgcagcgcacatgggagcaggctgttaaagggttaaatgaaagtaaaaaaccgttacagccaagtagctaa

SEQ ID NO: 146 - DNA, 741 bp, Pseudomonas aeruginosa; identical to SEQ ID NO: 145

SEQ ID NO: 147 - DNA, 741 bp, Pseudomonas aeruginosa; identical to SEQ ID NO: 145

SEQ ID NO: 148 - DNA, 741 bp, Pseudomonas aeruginosa; identical to SEQ ID NO: 145

SEQ ID NO: 149 - DNA, 738 bp, Pseudomonas aeruginosa
atgaaaaaaatatttgtgttatttgtatttttgttttgcagtattactgccgccggagagtctttgcctgatataaaaattgagaaacttgacgaagatgtttatgttcatacttcttttgaaaagataaccggctggggtgttattactaaacacggcttggtggttcttgtaaatactgatgcctatataattgacactccatttacagctaaagatactgaaaaattagtccgctggtttgtggggcgtggttataaaatcaaaggcagtatttcctcacattttcatagcgatagcgcaggtggaattgagtggcttaattctcaatctatccccacatatgcatctaaattaacaaatgagcttcttaaaaagaacggtaatgcgcaagccgaaaactcatttagtggcgttagctattggctagttaaacataaaattgaagttttctatccaggaccagggcacactcaggataatgtagtggtttggttgcctgaaaagaaaattttatttggcggttgttttattaagccggacggtcttggttatttgggagacgcaaatctagaagcatggcctaagtccgcagaaacattaatgtctaagtatggtaatgcaaaactggttgtttcgagtcatagtgaaattgggggcgcatcactattgaagcgcacttgggagcaggctgttaaggggctaaaagaaagtaaaaaccatcacagccccaaataa

SEQ ID NO: 150 - DNA, 741 bp, Klebsiella pneumoniae
atgaaaaaattatttgttttatgtgtattcttcttctgcaacattgcagttgcagaagaatctttgcctgatttaaaaattgagaagcttgaagaaggcgtttatgttcatacttcgtttgaagaagttaaaggttggagtgtggtcactaaacacggtttggtggttcttgtgaaaaatgacgcctatctgattgatactccaattactgctaaagatactgaaaaattagtcaattggtttgttgagcggggctataaaatcaaaggcagtatttccacacatttccatggtgacagtacggctggaatagagtggcttaattctcaatctatccccacatatgcttctgaattaacaaatgaacttcttaaaaaagacaataaggtacaagctaaacactctttttatggggttagttattcactaataaaaaacaaaattgaagttttttatccaggcccagggcacactcaagataacgtagtggtttggttacctgaaaagaaaattttattcggtggttgctttgttaaaccggacggtcttggctatttgggggacgcaaatttagaagcttggccaaagtccgctaaaatattaatgtctaaatatggtaaagcaaaactagttgtgtcgagtcatagtgatattggagatgtatcactcttgaaacgtacatgggagcaggctgttaaagggctgaatgaaagtaaaaaatcatcacagccaagcgactaa

SEQ ID NO: 151 - DNA, 741 bp, Acinetobacter baumannii
atgaaaaaattatttgttttatgtgtattcttcttctgcaacattgcagttgcagaagaatctttgcctgatttaaaaattgagaagcttgaagaaggcgtttatgttcatacttcgtttgaagaagttaaaggttggagtgtggtcactaaacacggtttggtggttcttgtgaaaaatgacgcctatctgattgatactccaattactgctaaagatactgaaaaattagtcaattggtttgttgagcggggctataaaatcaaaggcagtatttccacacatttccatggtgacagtacggctggaatagagtggcttaattctcaatctatccccacatatgcttctgaattaacaaatgaacttcttaaaaaagacaataaggtacaagctaaacactcttttaatggggttagttattcactaattaaaaacaaaattgaagttttttatccaggcccagggcacactcaagataacgtagtggtttggttacctgaaaagaaaattttattcggtggttgctttgttaaaccggacggtcttggctatttgggggacgcaaatttagaagcttggccaaagtccgctaaaatattaatgtctaaatatggtaaagcaaaactagttgtgtcgagtcatagtgatattggagatgtatcactcttgaaacgtacatgggagcaggctgttaaagggctgaatgaaagtaaaaaatcatcacagccaagcgactaa

SEQ ID NO: 152 - DNA, 741 bp, Pseudomonas aeruginosa; identical to SEQ ID NO: 151

SEQ ID NO: 153 - DNA, 741 bp, Pseudomonas aeruginosa
atgaaaaaattatttgttttatgtgtattcttcttctgcaacattgcagttgcagaagaatctttgcctgatttaaaaattgagaagcttgaagaaggcgtttatgttcatacttcgtttgaagaagttaaaggttggagtgtggtcactaaacacggtttggtggttcttgtgaaaaatgacgcctatctgattgatactccaattactgctaaagatactgaaaaattagtcaattggtttgttgagcggggctataaaatcaaaggcagtatttcaacacatttccatggtgacagtacggctggaatagagtggcttaattctcaatctatccccacatatgcttctgaattaacaaatgaacttcttaaaaaagacaataaggtacaagctaaacactcttttaatggggttagttattcactaattaaaaacaaaattgaagttttttatccaggcccagggcacactcaagataacgtagtggtttggttacctgaaaagaaaattttattcggtggttgctttgttaaaccggacggtcttggctatttgggggacgcaaatttagaagcttggccaaagtccgctaaaatattaatgtctaaatatggtaaagcaaaactagttgtgtcgagtcatagtgatattggagatgtatcactcttgaaacgtacatgggagcaggctgttaaagggctgaatgaaagtaaaaaatcatcacagccaagcgactaa

SEQ ID NO: 154 - DNA, 738 bp, Pseudomonas aeruginosa
atgaaaaaactatttgttttatgtatatttttgttttgtagcattactgccgcaggagcgtctttgcctgatttaaaaattgagaagcttgaagagggtgtttatgttcatacatcgtttgaagaagttaacggctggggtgttgcttctaaacacggtttggtggttcttgtaaatactgacgcctatctgattgacactccatttactgctaaagatactgaaaagttagtcaattggtttgtggagcgcggctataaaatcaaaggcagtatttcctcacatttccatagcgacagcacgggtggaatagagtggcttaattctcaatctattcccacgtatgcatctgtattaacaaatgaacttctcaaaaaagacggtaaggtgcaagctaaaaactcatttagcggagttagctattggctagttaaaaataaaattgaagttttttatccaggcccagggcacactcaagataacgtagtggtttggctacctaaaaataaaatcttatttggtggttgttttgttaaaccatatggtcttggtaatctagatgacgcaaatgttgaagcatggccacattcggctgaaaaattaatatctaagtatggtaatgcaaaactggttgttccaagccatagtgacataggagatgcgtcgctcttgaagcttacgtgggaacaggcggtaaaagggcttaatgaaagcaaaaaaagtaacactgttcattaa

SEQ ID NO: 155 - DNA, 869 bp, Acinetobacter baumannii
atgaaaaaactatttgttttatgtatatttttgttttgtagcattactgccgcaggagcgtctttgcctgatttaaaaattgagaagcttgaagagggtgtttatgttcatacatcgtttgaagaagttaacggctggggtgttgtttctaaacacggtttggtggttcttgtaaatactgacgcctatctgattgacactccatttactgctaaagatactgaaaagttagtcaattggtttgtggagcgcggctataaaatcaaaggcagtatttcctcacatttccatagcgacagcacgggtggaatagagtggcttaattctcaatctattcccacgtatgcatctgtattaacaaatgaacttctcaaaaaagacggtaaggtgcaagctaaaaactcatttagcggagttagctattggctagttaaaaataaaattgaagttttttatccaggcccagggcacactcaagataacgtagtggtttggctacctaaaaataaaatcttatttggtggttgttttgttaaaccatatggtcttggtaatctagatgacgcaaatgttgaagcatggccacattcggctgaaaaattaatatctaagtatggtaatgcaaaactggttgttccaagccatagtgacataggagatgcgtcgctcttgaagcttacgtgggaacaggcggtaaaagggcttaatgaaagcaaaaaaagtaacactgttcattaaccaaatttctaacaagtcgctgaagcatcgcaccttcggtgctggacagtttttaagtcgcgcttttgtggttttgctacgcaaaaggtttccacaaaatcacaacttaaaaactgccgcttagctcggcg

SEQ ID NO: 156 - DNA, 738 bp, Pseudomonas aeruginosa
atgaaaaaactatttgttttatgtatatttttgttttgtagcattactgccgcaggagcgtctttgcctgatttaaaaattgagaagcttgaagagggtgtttatgttcatacatcgtttgaagaagttaacggctggggtgttttttctaaacacggtttggtggttcttgtaaatactgacgcctatctgattgacactccatctactgctaaagatactgaaaagttagtcaattggtttgtggagcgcggctataaaatcaaaggcagtatttcctcacatttccatagcgacagcacgggtggaatagagtggcttaattctcaatctattcccacgtatgcatctgtattaacaaatgaacttctcaaaaaagacggtaaggtgcaagctaaaaactcatttagcggagttagctattggctagttaaaaataaaattgaagttttttatccaggcccagggcacactcaagataacgtagtggtttggctacctaaaaataaaatcttatttggtggttgttttgttaaaccatatggtcttggtaatctagatgacgcaaatgttgaagcatggccacattcggctgaaaaattaatatctaagtatggtaatgcaaaactggttgttccaagccatagtgacataggagatgcgtcgctcttgaagcttacgtgggaacaggcggtaaaagggcttaatgaaagcaaaaaaagtaacactgttcattaa

SEQ ID NO: 157 - DNA, 738 bp, Pseudomonas aeruginosa
atgaaaaaactatttgttttatgtatatttttgttttgtagcattactgccgcaggagcgtctttgcctgatttaaaaattgagaagcttgaagagggtgtttatgttcatacatcgtttgaagaagttaacggctggggtgttttttctaaacacggtttggtggttcttgtaaatactgacgcctatctgattgacactccatttactgctaaagatactgaaaagttagtcaattggtttgtggagcgcggctataaaatcaaaggcagtatttcctcacatttccatagcgacagcacgggtggaatagagtggcttaattctcaatctattcccacgtatgcatctgtattaacaaatgaacttctcaaaaaagacggtaaggtgcaagctaaaaactcatttagcggagttagctattggctagttaaaaataaaattgaagttttttatccaggcccagggcacactcaagataacgtagtggtttggctacctaaaaataaaatcttatttggtggttgttttgttaaaccatatggtcttggtaatctagatgacgcaaatgttgaagcatggccacattcggctgaaaaattaatatctaagtatggtaatgcaaaactggttgttccaagccatagtgacataggagatgcgtcgctcttgaagcttacgtgggaacaggcggtaaaagggcttaatgaaagcaaaaaaagtaacactgttcattaa

SEQ ID NO: 158 - DNA, 741 bp, Pseudomonas fluorescens
atgaagaaattatttgttttatgtgtgtttttgttttgtagcattactgccgcaggagagtctttgcccgatttaaaaattgaaaagcttgaagaaggtgtttatgttcatacatcgtttgaagaagttaatggttggggcgttgtttctaaacacggtttggttattcttgtgaatactgacgcctatctgattgacactccattcacggctaaagatactgaaaagttagtcacctggtttgtggagcgcggctataaaatcaaaggtagcatttcctcacatttccatagcgacagcacgggtggaatagagtggcttaattctcaatcaattcccacgtatgcatctgaattaacaaatgaccttcttaaacaaaacggtaaggtacaagctaaaaactcatttagcggagttagttattggttagttaaaaataaaattgaagttttctatcccggccccgggcacactcaagataacgtagtggtttggttgcctgaaaagaaaattttatttggtgggtgctttgttaaaccgtacggtcttggaaatctcgatgacgcaaatgttgtagcatggccacattctgctgaaatattaatgtctaggtatggtaatgcaaaactggttgttccaagccatagtgacatcggagatgcgtcgctcttgaagcttacatgggagcaggctgttaaagggctaaaagaaagtaaaaaaccatcagagccaagtaactaa

SEQ ID NO: 159 - DNA, 741 bp, Pseudomonas aeruginosa
atgaaaaaattatttgttttatgtatctttttgttttgtagcattactgccgcaggagagtctttgcctgatttaaaaattgagaagcttgaagacggtgtttatgttcatacatcgtttgaagaagttaacggttggggtgttgttactaaacacggtttggtgtttcttgtaaacacagacgcctatctgattgacactccatttgctgctaaagacactgaaaagttagtaaattggtttgtggagcgcggttataaaataaaaggcagtatttcctcacattttcatagcgacagctcgggtggaatagaatggcttaactctcaatctattcccacgtatgcatctgaattaacaaacgaacttcttaaaaagaacggtaaggtgcaagctaaaaactcatttagcggagttagttattggctacttaaaaataaaattgaaattttttatccgggccctgggcacactcaagataacgtagtggtttggttgcctgaaaagaaaattttatttggtgggtgttttgttaaaccgtacggtcttggaaatctcgatgatgcaaatgttgaagcgtggccacattctgctgaaatattaatgtctaggtatggtaatgcaaaactggttgttccaagccatagtgacgtcggagatgcgtcgctcttgaagcttacatgggagcaggctgttaaagggctaaaagaaagtaaaaaaccatcacagccaagtaactaa

SEQ ID NO: 160 - DNA, 738 bp, Pseudomonas aeruginosa
atgagcaagttatttgtattctttatgtttttgttttgtagcattactgccgcaggagagtctttgccagatttaaaaattgagaagcttgacgaaggcgtttatgttcatacttcgtttgaagaagttaacggttggggtgttattcctaaacacggcttggtggttcttgtaaatactgatgcctatctgatagacactccatttactgctaaagatactgaaaatttagttaattggtttgttgagcgcggctatagaataaaaggcagtatttcctcacatttccatagcgacagcacgggtggaatagagtggcttaattctcaatctatccccacgtatgcatctgaattaacaaatgaacttcttaaaaaagacggtaaggtacaagctaaatattcatttagcggagttagctattggctagttaagaaaaagattgaagttttttatcctggtccagggcacgctccagataacgtagtggtttggctgcctgaaaatagagttttgttcggtggttgttttgttaaaccctacggtctaggtaatttgggtgacgcaaatttagaagcttggccaaaatccgccaaattattaatgtcaaaatatagtaaggcaaaactggttgtaccaagtcatagtgacataggagattcgtcgctcttgaagcttacatgggagcagacggtaaaaggattcaatgaaagcaaaaaaagtaccactgcacattaa

SEQ ID NO: 161 - DNA, 738 bp, Pseudomonas aeruginosa
atgagcaagttatttgtattctttatgtttttgttttgtagcattactgccgcaggagagtctttgccagatttaaaaattgagaagcttgacgaaggcgtttatgttcatacttcgtttgaagaagttaacggttggggtgttattcctaaacacggcttggtggttcttgtaaatactgatgcctatctgatagacactccatttactgctaaagatactgaaaatttagttaattggtttgttgagcgcggctatagaataaaaggcagtatttcctcacatttccatagcgacagcacgggtggaatagagtggcttaattctcaatctatccccacgtatgcatctgaattaacaaatgaacttcttaaaaaagacggtaaggtacaagctaaatattcatttagcggagttagctattggctagttaagaaaaagattgaagttttttatcctggttcagggcacgctccagataacgtagtggtttggctgcctgaaaatagagttttgttcggtggttgttttgttaaaccctacggtctaggtaatttgggtgacgcaaatttagaagcttggccaaaatccgccaaattattaatgtcaaaatatagtaaggcaaaactggttgtaccaggtcatagtgacataggagattcgtcgctcttgaagcttacatgggagcagacggtaaaaggattcaatgaaagcaaaaaaagtaccactgcacattaa

SEQ ID NO: 162 - DNA, 738 bp, Pseudomonas aeruginosa
atgagcaagttatttgtattctttatgtttttgttttgtagcattactgccgcaggagagtctttgccagatttaaaaattgagaagcttgacgaaggcgtttatgttcatacttcgtttgaagaagttaacggttggggtgttattcctaaacacggcttggtggttcttgtaaatactgatgcctatctgatagacactccatttactgctaaagatactgaaaatttagttaattggtttgttgagcgcggctatagaataaaaggcagtatttcctcacatttccatagcgacagcacgggtggaatagagtggcttaattctcaatctatccccacgtatgcatctgaattaacaaatgaacttcttaaaaaagacggtaaggtacaagctaaatattcatttagcggagttagctattggctagttaagaaaaagattgaagttttttatcctggtccagggcacgctccagataacgtagtggtttggctgcctgaaaatagagttttgttcggtggttgttttgttaaaccctacggtctaggtaatttgggtgacgcaaatttagaagcttggccaaaatccgccaaattattaatgtcaaaatatagtaaggcaaaactggttgtaccaggtcatagtgacataggagattcgtcgctcttgaagcttacatgggagcagacggtaaaaggattcaatgaaagcaaaaaaagtaccactgcacattaa

SEQ ID NO: 163 - DNA, 741 bp, Pseudomonas aeruginosa
atgagcaagttatttgtattccttatttttttgttttgtagcattactgccgcagcagagtctttgccagatttaaaaattgagaagctcgacgaaggcgtttatgttcatacttcgtttgaagaagttaacggttggggtgttgttcctaaacatggcttggtggttcttgtaaatactgaggcctatctgattgacactccatttacggctaaagatactgaaaagttagtcacttggtttgtggaacgcggctataaaataaaaggcagtatttcctctcattttcatagcgacagcacaggcggaatagagtggcttaattctcaatctatccccacgtatgcatctgaattaacaaatgaacttcttaaaaaaggcggtaaagtacaagctaaaaattcatttagcggagttagctattggctagttaagaaaaagattgaagttttttatcctggtccagggcacactccagataacgtagtggtttggctacctgaaaatagagttttgttcggtggttgttttgttaaaccgtacggtcttggaaatctcgatgacgcaaatgttgaagcatggccacattctgctgaaatattaatgtctaggtatggtaatgcaaaactggttgttccaagccatagtgacatcggaaatgcgtcgctcttgaagcttacatgggagcaggctgttaaagggctaaaagaaagtaaaaaaccatcacagccaagtaactaa

SEQ ID NO: 164 - DNA, 741 bp, Pseudomonas aeruginosa
atgaacaagttatctgtattctttatgtttatgttttgtagcattactgccgcaggagagtctttgccagatttaaaaattgagaagcttgacgaaggtgtttatgttcatacttcgtttgaagaagttaacggttggggtgttgttcctaaacacggcttggtggttcttgtaaatactgaggcctatctgattgacactccatttacggcaaaagatactgaaaagttagtcacttggtttgtggagcgcggctataaaataaaaggcagtatttcctctcattttcatagcgacagcacgggcggaatagagtggcttaattctcaatctatccccacgtatgcatctgaattaacaaatgaacttcttaaaaaagacggtaaggtacaagctaaaaattcatttagcggaggtagctattggctagttaataataagattgaagttttttatcctggtccagggcacactccagataacgtagtggtttggctacctgaaaatagagttttgttcggtggttgttttgttaaaccgtacggtcttggtaatttgggtgacgcaaatttagaagcttggccaaagtccgccaaaatattaatgtctaaatatggtaaagcaaagttggttgtttcaagtcatagtgaaactgggaacgcatcactcttgaaacttacttgggagcaggctgttaaagggctaaaagaaagtaaaaaaccatcactgccaagtaactaa

SEQ ID NO: 165 - DNA, 741 bp, Pseudomonas aeruginosa; identical to SEQ ID NO: 164

SEQ ID NO: 166 - DNA, 741 bp, Acinetobacter baumannii
atgagcaagttatttgtattctttatgtttttgttttgtagcattactgccgcagcagagtctttgccagatttaaaaattgagaagcttgacgaaggcgtttatgttcatacttcgtttgaagaagttaacggttggggtgttgttcctaaacacggcttggtggttcttgtaaatactgaggcctatctgattgacactccatttacggctaaagatactgaaaagttagtcacttggtttgtggaacgcggctataaaataaaaggcagtatttcctctcattttcatagcgacagcacgggcggaatagagtggcttaattctcaatctatccccacgtatgcatctgaattaacaaatgaacttcttaaaaaagacggtaaagtacaagctaaaaattcatttagcggagctagctattggctagttaagaaaaagattgaagttttttatcctggtccagggcacactccagataacgtagtggtttggctacctgaaaatagagttttgttcggtggttgttttgttaaaccgtacggtctaggtaatttgggtgacgcaaatgtagaagcttggccaaagtccgccaaattattaatgtccaaatatggtaaggcaaaactggtagttccaagtcacagtgaagttggagacgcatcactcttgaaacgtacgttagaacaggcggttaaagggttaaacgaaagtaaaaaaccatcaaaaccaagtaactaa

SEQ ID NO: 167 - DNA, 741 bp, Acinetobacter baumannii; identical to SEQ ID NO: 166

SEQ ID NO: 168 - DNA, 741 bp, Klebsiella oxytoca
atgagcaagttatttgtattctttatgtttttgttttgtagcattactgccgcagcagagtctttgccagatttaaaaattgagaggcttgatgaaggcgtttatgttcatacttcgtttgaagaagttaacggttggggtgttgttcctaaacacggcttggtggttcttgtaaatactgaggcctatctgattgacactccatttacggctaaagatactgaaaagttagtcacttggtttgtgggacgcggctataaaataaaaggcagtatttcctctcattttcatagcgacagcacgggcggaatagagtggcttaattctcaatctatccccacgtatgcatctgaattaacaaatgaacttcttaaaaaagacggtaaggtacaagctaaaaattcatttggcggagttagctattggctagttaagaataagattgaagttttttatcctggtccagggcacactccagataacgtagtggtttggctacctgaaaatagagttttgttcggtggttgttttgttaaaccgtacggtcttggtaatttgggtgacgcaaatttagaagcttggccaaagtccgccaaattattaatgtccaaatatggtaaggcaaaactggttgttccaagtcacagtgaagttggagacgcatcactcttgaagcgaacattagaacatgcggttaaagggttaaatgaaagtaaaaaaccatcaaaaccaagtaactaa

SEQ ID NO: 169 - DNA, 543 bp, Pseudomonas aeruginosa
acttcgtttgaggaagttaacggctggggcgtggttcctaaacacggcttggtggttcttgtaaatactgacgcttatttgattgacactccatttacagctaaagatactgaaaagttagttacttggtttgtagagcgcggctataaaataaaaggcagtatctcctctcattttcatagcgacagcacgggcggaatagagtggcttaattctcaatctattccaacatatgcatctgaattaacaaatgaacttcttaaaaaagacggtaaggtacaagctaaaaattcatttagcggagccagctattggttagttaagaaaaagattgaaattttttatcctggcccagggcacactccagataacgtagtggtttggctacctgaacatagagttttgtttggtggttgttttgttaaaccgtatggtctaggtaatttgggtgacgcaaatttagaagcttggccaaagtctgccaaattattagtgtccaaatatggtaaggcaaaactggttccaagtcacagtgaagttgga

SEQ ID NO: 170 - DNA, 741 bp, Pseudomonas aeruginosa
atgaaaaagttatcagtattctttatgtttttgttttgtagcattgctgcctcaggagaggctttgccagatttaaaaattgagaagcttgacgaaggcgtttatgttcatacttcgtttgaggaagttaacggctggggcgtgtttcctaaacacggcttggtggttcttgtaaatactgacgcttatttgattgacactccatttacagctaaagatactgaaaagttagttacttggtttgtagagcgcggctataaaataaaaggcagtatctcctctcattttcatagcgacagcacgggcggaatagagtggcttaattctcaatctattccaacatatgcatctgaattaacaaatgaacttcttaaaaaagacggtaaggtacaagctaaaaattcatttagcggagccagctattggttagttaagaaaaagattgaaattttttatcctggcccagggcacactccagataacgtagtggtttggctacctgaacatagagttttgtttggtggttgttttgttaaaccgtatggtctaggtaatttgggtgacgcaaatttagaagcttggccaaagtctgccaaattattagtgtccaaatatggtaaggcaaaactggttgttccaagtcacagtgaagttggagatgcatcactcttgaaacgtacattagaacaggctgttaaaggattaaacgaaagtaaaaagctatcaaaaccaagtaactaa

SEQ ID NO: 171 - DNA, 741 bp, Pseudomonas aeruginosa
atgaaaaagttatcagtattctttatgtttttgttttgtagcattgctgcctcaggagaggctttgccagatttaaaaattgagaagcttgacgaaggcgtttatgttcatacttcgtttgaggaagttaacggctggggcgtggttcctaaacacggcttggtggttcttgtaaatactgacgcttatttgattgacactccatttacagctaaagatactgaaaagttagttacttggtttgtagagcgcggctataaaataaaaggcagtatctcctctcattttcatagcgacagcacgggcggaatagagtggcttaattctcaatctattccaacatatgcatctgaattaacaaatgaacttcttaaaaaagacggtaaggtacaagctaaaaattcatttagcggagccagctattggttagttaagaaaaagattgaaattttttatcctggcccagggcacactccagataacgtagtggtttggctacctgaacatagagttttgtttggtggttgttttgttaaaccgtatggtctaggtaatttgggtgacgcaaatttagaagcttggccaaagtctgccaaattattagtgtccaaatatggtaaggcaaaactggttgttccaagtcacagtgaagttggagatgcatcactcttgaaacgtacattagaacaggctgttaaaggattaaacgaaagtaaaaagctatcaaaaccaagtaactaa

SEQ ID NO: 172 - DNA, 741 bp, Proteus mirabilis
atgaaaaaattatttgttttatgtgtctttgtcttttgtagtattactgtcgcaggtgagactttgcctaatttgagagttgaaaagcttgaagaaggtgtttatgttcatacatcgtatgaagaagttaaaggttggggtgttgttactaaacacggtttggtggttctcataggcgctgacgcctatctgattgatactccatttactgctaaagatactgaaaagttagtcaattggtttgtggagcgcggctataaaataaaaggcactgtttcctcacatttccatagcgacagtacggggggaatagagtggcttaactctcagtctatccccacgtatgcgtctgaattaacgaatgaacttctgaaaaaagacggtaaggttcaagccaaaaactcatttgacggggttagttattggctggcgaaagataaaatagaagtgttttatcctggccctggccacactcaagacaacgtagtagtttggctgcctgaaaaggaaatattatttggcggttgctttgttaagcctcacggccttggtaatttgggtgacgcaaatttagaggcttggccagagtccgccaaaatattgatggaaaaatatggtaaagcaaagctggttgtttcaggtcatagcgaaaccggagacgcgacacacttgaagcgtacctgggagcaggctgttaaaggacttaaagaaagtaaaaagacattgcagccaagcaactaa

SEQ ID NO: 173 - DNA, 741 bp, Acinetobacter calcoaceticus
atgagcaagttatctgtattctttatatttttgttttgtagcattgctaccgcagcagagcctttgccagatttaaaaattgaaaaacttgatgaaggcgtttatgttcatacttcgtttgaagaagttaacgggtggggcgtttttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaagtcacagtgaagctggagacgcatcactcttgaaacttacattagagcaggcggttaaagggttaaacgaaagtaaaaaaccatcaaaactaagcaattaa

SEQ ID NO: 174 - DNA, 535 bp, Pseudomonas aeruginosa
aagaagttaacgggtggggcgtttttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaagtcacagtgaagctgg

SEQ ID NO: 175 - DNA, 741 bp, Pseudomonas aeruginosa
atgagcaagttatctgtattctttatatttttgttttgtagcattgctaccgcagcagagcctttgccagatttaaaaattgaaaaacttgatgaaggcgtttatgttcatacttcgtttgaagaagttaacgggtggggcgtttttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaagtcacagtgaagctggagacgcatcactcttgaaacttacattagagcaggcggttaaagggttaaacgaaagtaaaaaaccatcaaaactaagcaactaa

SEQ ID NO: 176 - DNA, 741 bp, Klebsiella pneumoniae
atgagcaagttatctgtattctttatatttttgttttgtagcattgctaccgcagcagagcctttgccagatttaaaaattgaaaaacttgatgaaggcgtttatgttcatacttcgtttgaagaagttaacgggtggggcgttgttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaggtcacagtgaagctggagacgcatcactcttgaaacttacattagagcaggcggttaaagggttaaacgaaagtaaaaaaccatcaaaactaagcaactaa

SEQ ID NO: 177 - DNA, 741 bp, Klebsiella pneumoniae
atgagcaagttatctgtattctttatatttttgttttgtagcattgctaccgcagcagagtctttgccagatttaaaaattgaaaaacttgatgaaggcgtttatgttcatacttcgtttgaagaagttaacgggtggggcgttgttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaagtcacagtgaagctggagacgcatcactcttgaaacttacattagagcaggcggttaaagggttaaacgaaagtaaaaaaccatcaaaactaagcaactaa

SEQ ID NO: 178 - DNA, 741 bp, Citrobacter youngae
atgagcaagttatctgtattctttatatttttgttttgtagcattgctaccgcagcagagcctttgccagatttaaaaattgaaaaacttgatgaaggcgtttatgttcatacttcgtttgaagaagttaacgggtggggcgttgttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaagtcacagtgaagctggagacgcatcactcttgaaacttacattagagcaggcggttaaagggttaaacgaaagtaaaaaaccatcaaaactaagcaactaa

SEQ ID NO: 179 - DNA, 741 bp, Acinetobacter baumannii; identical to SEQ ID NO: 178

SEQ ID NO: 180 - DNA, 741 bp, Pseudomonas putida
atgaagaaattatttgttttatgcatttttttgtttttaagtattactgcctcaggtgaggttttgcctgatttgaaaattgagaagcttgaagagggtgtttatcttcatacatcttttgaagaggttagcggttggggtgttgttactaaacatggtttggtagttcttgtaaataatgacgcctatctaattgacactccatttacaaataaagatactgaaaaattagttgcttggtttgtagggcgcggctttacaataaagggaagtgtttcctcacattttcatagcgacagtacgggtggaatagagtggcttaattctcaatctattcccacgtatgcatctgagttaacaaatgaacttctgaaaaagaacggtaaggtgcaagctacaaattcatttagcggggttagttattggctagttaaaaataaaattgaaattttttatcccggcccaggacatactcaagataacgtagtggtttggctacctgaaaacaaaattttattcggtggttgttttgttaaaccggacggtcttggtaatttggatgacgcaaatttaaaagcttggccaaagtccgcaaaaatattaatgtctaaatatggtaaagcaaagttagttgtttcaggtcatagtgaaattgggaacgcatcactcttgaaacttacttgggagcaggctgttaaagggctaaaagaaagtaaaaaaccattactgccaagtaactaa

SEQ ID NO: 181 - DNA, 741 bp, Acinetobacter baumannii
atgaagaaattatttgttttatgtgtatgcttcctttgtagcattactgccgcgggagcgcgtttgcctgatttaaaaatcgagaagcttgaagaaggtgtttatgttcatacatcgttcgaagaagttaacggttggggtgttgtttctaaacacggtttggtggttcttgtaaacactgacgcctatctgattgacactccatttactgctacagatactgaaaagttagtcaattggtttgtggagcgcggctataaaatcaaaggcactatttcctcacatttccatagcgacagcacagggggaatagagtggcttaattctcaatctattcccacgtatgcatctgaattaacaaatgaacttcttaaaaaagacggtaaggtgcaagctaaaaactcatttagcggagttagttattggctagttaaaaataaaattgaagttttttatcccggcccggggcacactcaagataacgtagtggtttggttacctgaaaagaaaattttattcggtggttgttttgttaaaccggacggtcttggtaatttgggtgacgcaaatttagaagcttggccaaagtccgccaaaatattaatgtctaaatatgttaaagcaaaactggttgtttcaagtcatagtgaaattggggacgcatcactcttgaaacgtacatgggaacaggctgttaaagggctaaatgaaagtaaaaaaccatcacagccaagtaactaa

SEQ ID NO: 182 - DNA, 741 bp, Serratia marcescens
atgaagaaattatttgttttatgtgtatgcttcctttgtagcattactgccgcaggagcggctttgcctgatttaaaaatcgagaagcttgaagaaggtgtttatgttcatacatcgttcgaagaagttaacggttggggtgttgtttctaaacacggtttggtggttcttgtaaacactgacgcctatctgattgacactccatttactgctacagatactgaaaagttagtcaattggtttgtggagcgcggctataaaatcaaaggcactatttcctcacatttccatagcgacagcacagggggaatagagtggcttaattctcaatctattcccacgtatgcatctgaattaacaaatgaacttcttaaaaaagacggtaaggtgcaagctaaaaactcatttagcggagttagttattggctagttaaaaataaaattgaagttttttatcccggcccggggcacactcaagataacgtagtggtttggttacctgaaaagaaaattttattcggtggttgttttgttaaaccggacggtcttggtaatttgggtgacgcaaatttagaagcttggccaaagtccgccaaaatattaatgtctaaatatggtaaagcaaaactggttgtttcaagtcatagtgaaattggggacgcatcactcttgaaacgtacatgggaacaggctgttaaagggctaaatgaaagtagaaaaccatcacagccaagtaactaa

SEQ ID NO: 183 - DNA, 741 bp, Pseudomonas aeruginosa
atgaagaaattatttgttttatgtgtatgcttcctttgtagcattactgccgcaggagcggctttgcctgatttaaaaatcgagaagcttgaagaaggtgtttatgttcatacatcgttcgaagaagttaacggttggggtgttttttctaaacacggtttggtggttcttgtaaacactgacgcctatctgattgacactccatttactgctacagatactgaaaagttagtcaattggtttgtggagcgcggctataaaatcaaaggcactatttcctcacatttccatagcgacagcacagggggaatagagtggcttaattctcaatctattcccacgtatgcatctgaattaacaaatgaacttcttaaaaaagacggtaaggtgcaagctaaaaactcatttagcggagttagttattggctagttaaaaataaaattgaagttttttatcccggcccggggcacactcaagataacgtagtggtttggttacctgaaaagaaaattttattcggtggttgttttgttaaaccggacggtcttggtaatttgggtgacgcaaatttagaagcttggccaaagtccgccaaaatattaatgtctaaatatgttaaagcaaaactggttgtttcaagtcatagtgaaattggggacgcatcactcttgaaacgtacatgggaacaggctgttaaagggctaaatgaaagtaaaaaaccatcacagccaagtaactaa

SEQ ID NO: 184 - DNA, 741 bp, Aeromonas punctata
atgaagaaattatttgttttatgtgtatgcttcctttgtagcattactgccgcaggagcggctttgcctgatttaaaaatcgagaagcttgaagaaggtgtttatgttcatacatcgttcgaagaagttaacggttggggtgttgtttctaaacacggtttggtggttcttgtaaacactgacgcctatctgattgacactccatttactgctacagatactgaaaagttagtcaattggtttgtggagcgcggctataaaatcaaaggcactatttcctcacatttccatagcgacagcacagggggaatagagtggcttaattctcaatctattcccacgtatgcatctgaattaacaaatgaacttcttaaaaaagacggtaaggtgcaagctaaaaactcatttagcggagttagttattggctagttaaaaataaaattgaagttttttatcccggcccggggcacactcaagataacgtagtggtttggttacctgaaaagaaaattttattcggtggttgttttgttaaaccggacggtcttggtaatttgggtgacgcaaatttagaagcttggccaaagtccgccaaaatattaatgtctaaatatgttaaagcaaaactggttgtttcaagtcatagtgaaattggggacgcatcactcttgaaacgtacatgggaacaggctgttaaagggctaaatgaaagtaaaaaaccatcacagccaagtaactaa

SEQ ID NO: 185 - DNA, 741 bp, Serratia marcescens
atgaagaaattatttgttttatgtgtatgcttcctttgtagcattactgccgcaggagcggctttgcctgatttaaaaatcgagaagcttgaagaaggtgtttatgttcatacatcgttcgaagaagttaacggttggggtgttgtttctaaacacggtttggtggttcttgtaaacactgacgcctatctgattgacactccatttactgctacagatactgaaaagttagtcaattggtttgtggagcgcggctataaaatcaaaggcactatttcctcacatttccatagcgacagcacagggggaatagagtggcttaattctcaatctattcccacgtatgcatctgaattaacaaatgaacttcttaaaaaagacggtaaggtgcaagctaaaaactcatttagcggagttagttattggctagttaaaaataaaattgaagttttttatcccggcccggggcacactcaagataacgtagtggtttggttacctgaaaagaaaattttattcggtggttgttttgttaaaccggacggtcttggtaatttgggtgacgcaaatttagaagcttggccaaagtccgccaaaatattaatgtctaaatatggtaaagcaaaactggttgtttcaagtcatagtgaaattggggacgcatcactcttgaaacgtacatgggaacaggctgttaaagggctaaatgaaagtaaaaaaccatcacagccaagtaactaa

SEQ ID NO: 186 - DNA, 741 bp, Klebsiella pneumoniae; identical to SEQ ID NO: 185

SEQ ID NO: 187 - DNA, 741 bp, Pseudomonas aeruginosa
atgaagaaattatttgttttatgtgtatgcttcttttgtagcattactgccgcaggatcgtctttacctgatttaaaaattgagaagcttgaagaaggtgtttttgttcatacatcgttcgaagaagttaacggttggggggttgttactaaacacggtttggtggtgcttgtaaacacagacgcctatctaattgacactccatttactgctacagacactgaaaaattagtcaattggtttgtggagcgcggctataaaatcaaaggcactatttcatcacatttccatagcgacagcacaggaggaatagagtggcttaattctcaatctattcccacgtatgcatctgaattaacaaatgaacttttgaaaaaatccggtaaggtacaagctaaatattcatttagcgaagttagctattggctagttaaaaataaaattgaagttttttatcctggcccaggtcacactcaagataacctagtggtttggttgcctgaaagtaaaattttattcggtggttgctttgttaaacctcacggtcttggcaatttaggtgacgcaaatttagaagcttggccaaagtccgccaaaatattaatgtctaaatatggcaaagcaaagcttgttgtttcaagtcatagtgagaaaggggacgcatcactattgaaacgtacatgggaacaagctcttaaagggcttaaagaaagtaaaaaaacatcatcaccaagtaactaa

SEQ ID NO: 188 - DNA, 747 bp, Pseudomonas aeruginosa
atgaagaaattatttgttttatgtgtatgcttcttttgtagcattactgccgcaggagcggctttacctgatttaaaaatcgagaagcttgaagaaggtgtttttgttcatacatcgttcgaagaggttaacggttggggggttgttactaaacacggtttagtggtgcttgtaaacacagacgcctatctaattgacactccatttactgctacagacactgaaaaattagtcaattggtttgtggagcgcggctatgaaatcaaaggcactatttcatcacatttccatagcgacagcacaggaggaatagagtggcttaattctcaatctattcccacgtatgcatctgaattaacaaatgaacttttgaaaaaatccggtaaggtacaagctaaatattcatttagcgaagttagctattggctagttaaaaataaaattgaagttttctaccctggcccaggtcacactcaagataacctagtggtttggttgcctgaaagtaaaattttattcggtggttgctttattaaacctcacggtcttggcaatttaggtgacgcaaatttagaagcttggccaaagtccgccaaaatattaatgtctaaatatggcaaagcaaagcttgttgtttcaagtcatagtgaaaaaggggacgcatcactaatgaaacgtacatgggaacaagcccttaaagggcttaaagaaagtaaaaaaacatcatcacaaagtacagcatcgtga

SEQ ID NO: 189 - DNA, 741 bp, Pseudomonas aeruginosa
atgaagaaattatttgttttatgtgtatgcttcttttgtagcattactgccgcaggagcggctttacctgatttaaaaatcgagaagcttgaagaaggtgtttttgttcatacatcgttcgaagaggttaacggttggggggttgttactaaacacggtttagtggtgcttgtaaacacagacgcctatctaattgacactccatttactgctacagacactgaaaaattagtcaattggtttgtggagcgcggctatgaaatcaaaggcactatttcatcacatttccatagcgacagcacaggaggaatagagtggcttaattctcaatctattcccacgtatgcatctgaattaacaaatgaacttttgaaaaaatccggtaaggtacaagctaaatattcatttagcgaagttagctattggctagttaaaaataaaattgaagttttctaccctggcccaggtcacactcaagataacctagtggtttggttgcctgaaagtaaaattttattcggtggttgctttattaaacctcacggtcttggcaatttaggtgacgcaaatttagaagcttggccaaagtccgccaaaatattaatgtctaaatatggcaaagcaaagcttgttgtttcaagtcatagtgaaaaaggggacgcatcactaatgaaacgtacatgggaacaagcccttaaagggcttaaagaaagtaaaaaaacatcatcaccaagtaactaa

SEQ ID NO: 190 - DNA, 741 bp, Pseudomonas aeruginosa; identical to SEQ ID NO: 189

SEQ ID NO: 191 - DNA, 741 bp, Pseudomonas aeruginosa
atgaagaaattatttgttttatgtgtatgcttcttttgtagcattactgccgcaggagcggctttacctgatttaaaaatcgagaagcttgaagaaggtgtttttgttcatacatcgttcgaagaggttaacggttggggggttgttactaaacacggtttagtggtgcttgtaaacacagacgcctatctaattgacactccatttactgctacagacactgaaaaattagtcaattggtttgtggagcgcggctatgaaatcaaaggcactatttcatcacatttccatagcgacagcacaggaggaatagagtggcttaattctcaatctattcccacgtatgcatctgaattaacaaatgaacttttgaaaaaatccggtaaggtacaagctaaatattcatttagcggagttagctattggctagttaaaaataaaattgaagttttctaccctggcccaggtcacactcaagataacctagtggtttggttgcctgaaagtgaaattttattcggtggttgctttattaaacctcacggtcttggcaatttaggtgacgcaaatttagaagcttggccaaagtccgccaaaatattaatgtctaaatatggcaaagcaaagcttgttgtttcaagtcatagtgaaaaaggggacgcatcactaatgaaacgtacatgggaacaagcccttaaagggcttaaagaaagtaaaaaaacatcatcaccaagtaactaa

SEQ ID NO: 192 - DNA, 741 bp, Klebsiella oxytoca
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgttgttcctaaacatggtttggtggttcttgtaaatgctgaggcttatttaattgacactccatttacggctaaagatactgaaaagttagtcacttggtttgtggagcgtggctataaaataaaaggcagtatttcctctcattttcatagcgacagcacgggcggaatagggtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggctgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaaggattaaacgaaagtaaaaaaccatcaaaaccaagcaactaa

SEQ ID NO: 193 - DNA, 741 bp, Shigella flexneri
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgttgttcctaaacatggtttggtggttcttgtaaatgctgaggcttatttaattgacactccatttacggctaaagatactgaaaagttagtcacttggtttgtggagcgtggctataaaataaaaggcagtatttcctctcattttcatagcgacagcacgggcggaatagggtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggctgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaggtcacagtgaagttggagacgcatcactcttgaaacttacattagagcaggcggttaaaggattaaacgaaagtaaaaaaccatcaaaaccaagcaactaa

SEQ ID NO: 194 - DNA, 741 bp, Acinetobacter baumannii
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgttgttcctaaacatggtttggtggttcttgtaaatgctgaggcttacctaattgacactccatttacggctaaagatactgaaaagttagtcacttggtttgtggagcctggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtactggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacgaaagtaaaaaaccatcaaaaccaagcaactaa

SEQ ID NO: 195 - DNA, 741 bp, Pseudomonas aeruginosa
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgttgttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgagtgacgcaaatatagaagcttggccaaagtccgccaaattattaaagtccaaatatggtaaggcaaaactggttgttccaggtcacagtgaagttggagacgcatcactcttgaaacttacattagagcaggcggttaaagggttaaacgaaagtaaaaaaccatcaaaaccaagcaactaa

SEQ ID NO: 196 - DNA, 741 bp, Acinetobacter soli
atgagcaagttatctgtattctttatatttttgttttgcagcattgctaccgcagcagagtctttgccagatttaaaaattgaaaagcttgatgaaggcgtttatgttcatacttcgtttgaagaagttaacaggtggggcgttgttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacgaaagtaaaaaaccatcaaaaccaagcaactaa

SEQ ID NO: 197 - DNA, 741 bp, Pseudomonas aeruginosa
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgtttttcctaaacatggtttggtggttcttgtaaatgctgaggcttacctaattgacactccatctacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacgaaagtaaaaaaccatcaaaaccaagcaactaa

SEQ ID NO: 198 - DNA, 741 bp, Pseudomonas aeruginosa
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgtttttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacgaaagtaaaaaaccatcaaaaccaagcaactaa

SEQ ID NO: 199 - DNA, 842 bp, Pseudomonas aeruginosa
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgtttttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacgaaagtaaaaaaccatcaaaaccaagcaactaaatttctaacaagtcgttgcagcacgccactacgtggctggacagtttgtaagttgcgcttttgtggtttgcttcgcaaagtattccacaacgcgcaactta

SEQ ID NO: 200 - DNA, 741 bp, Pseudomonas aeruginosa
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgttgttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaggtcacagtgaagttggagacgcatcactcttgaaacttacattagagcaggcggttaaagggttaaacgaaagtaaaaaaccatcaaaaccaagcaactaa

SEQ ID NO: 201 - DNA, 741 bp, Serratia marcescens; identical to SEQ ID NO: 200

SEQ ID NO: 202 - DNA, 741 bp, Pseudomonas aeruginosa
atgagcaagttatctgtattctttatatttttgttttgcagcattgctaccgcagcagagtctttgccagatttaaaaattgaaaagcttgatgaaggcgtttatgttcatacttcgtttaaagaagttaacgggtggggcgttgttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacgaaagtaaaaaaccatcaaaaccaagcaactaa

SEQ ID NO: 203 - DNA, 741 bp, Klebsiella oxytoca
atgagcaagttatctgtattctttatatttttgttttgcagcattgctaccgcagcagagtctttgccagatttaaaaattgaaaagcttgatgaaggcgtttatgttcatacttcgtttgaagaagttaacgggtggggcgttgttcctaaacatggtttggtggttcttgtaaatgctgaggcttacctaattgacactccatttacggctaaagatactgaaaagttagtcacttggtttgtggagcgtggctataaaataaaaggcagcatttcctctcattttcatagcgacagcacgggcggaatagagtggcttaattctcgatctatccccacgtatgcatctgaattaacaaatgaactgcttaaaaaagacggtaaggttcaagccacaaattcatttagcggagttaactattggctagttaaaaataaaattgaagttttttatccaggcccgggacacactccagataacgtagtggtttggttgcctgaaaggaaaatattattcggtggttgttttattaaaccgtacggtttaggcaatttgggtgacgcaaatatagaagcttggccaaagtccgccaaattattaaagtccaaatatggtaaggcaaaactggttgttccaagtcacagtgaagttggagacgcatcactcttgaaacttacattagagcaggcggttaaagggttaaacgaaagtaaaaaaccatcaaaaccaagcaactaa

SEQ ID NO: 204 - DNA, 741 bp, Serratia marcescens; identical to SEQ ID NO: 203

SEQ ID NO: 205 - DNA, 741 bp, Pseudomonas aeruginosa
atgaaaaaaatatttgtgttatttgtatttttgttttgcagtattactgccgccggagagtctttgcctgatataaaaattgagaaacttgacgaagatgtttatgttcatacttcttttgaagaagataacggctggggtgttattactaaacacggcttggtggttcttgtaaatactgatgcctatataattgacactccatttacagctaaagatactgaaaaattagtccgctggtttgtggggcgtggttataaaatcaaaggcagtatttcctcacattttcatagcgatagcgcaggtggaattgagtggcttaattctcaatctatccccacatatgcatctaaattaacaaatgagcttcttaaaaagaacggtaatgcgcaagccgaaaactcatttagtggcgttagctattggctagttaaacataaaattgaagttttctatccaggaccagggcacactcaggataatgtagtggtttggttgcctgaaaagaaaattttatttggcggttgttttattaagccggacggtcttggttatttgggagacgcaaatctagaagcatggcctaagtccgcagaaacattaatgtctaagtatggtaatgcaaaactggttgtttcgagtcatagtgaaattgggggcgcatcactattgaagcgcacttgggagcaggctgttaaggggctaaaagaaagtaaaaaaccatcacagccaaataactaa

SEQ ID NO: 206 - DNA, 2572 bp, Enterobacter cloacae
atatgttgaatcatttatgagatttaaaaaacatcacttcttaggattaaaagttctttattactatttttatttacagcttgattattatcaagttagtgaaatcttaaacattgattcaaaaaaaattagccggcatatcatcagatgtcttacaacagtcaattataaaggtaagagaagtttttggtacgattacttaaataataagcattgatatatctttagcagagattaggatttaagtacaaactctctgctatgctcgataatccattccttaaaaacattaagtgcttcactattatgattaacccgagatttctgtaaagtcagccaataaccgccaagcgtggcataaatttcaaaaggttttgctacggagcgctcatttaattctttttgaaacatatgataaggaactaatgctgcataatcacctaattttacagcatctatcatatgtctggtagaatcaaagataggcccgttaacagtccaagagtcgatcccagcagctttaaaccaattattccattcatcctttcgataggagcgcagtaagcaaactttttttaagtctgatggaatgcacagtttcttggatgtttccggggaacataatactgtatgcggcgcctgaaagagtaaaatattttccgtattaatccagccgcctgatccaaatctaatagatgcatcaataccttctgcagctagattgacaacgttattatgcgttaaaattctaatatcaatccatggattttcattaaggaactcagtaatccgaggaagaagccaacctattgcaaaagtcccaacgacagcaatagtaagcacttctttaatcttaccttcatggaatttatccatggcagaactcatcatctgcaaagcttctgtaatcgaaggcaaaagatgcaggccttcgtctgtcaactgtaacccacgcggtactcttgtaaaaagagccacaccaagcatctgttcaagtgttcttacctgctggcttacggcggcctggcttacatgaagctctagtccggctttagtaaaatttagatatcttgcagaagcttcaaaagctcgtaatgcatttaaaggtaatctggcacgcatagtaacccaaagaaaaagttatggctgtataaaaattatattgcttcaaagaaaaaataacaactgtataatcgccctctgttgaaagttaatacccgccatgtcctagttaatatagaaaattgtatataaggtaaaaccatgtcacttaatgtaaaaccaagtagaatagccatcttgtttagctcttgtttagtttcaatatcatttttctcacaggccaatacaaagggcatcgatgagattaaagaccttgaaacagatttcaatggtagaattggtgtctacgctttagacactggctcaggcaaatcattttcatacaaagcaaatgaacgatttccattatgtagttctttcaaaggttttttagctgctgctgtattaaaaggctctcaagataatcaactaaatcttaatcagatcgtgaattataatacaagaagtttagagttccattcacccatcacaactaaatataaagataatggaatgtcattaggtgatatggctgctgcagctttacaatatagcgacaatggtgctactaatattatccttgaacgatatatcggtggtcctgagggtatgactaaattcatgcggtcgattggagataaagattttagactcgatcgttgggagttagatctaaacacagctattcctggcgatgaacgtgacacatctacacctgcagcagtagctaagagcctgaaaacccttgcactgggtaacatactcaatgagcgtgaaaaggaaacctatcagacatggttaaagggtaacacaaccggtgcagcgcgtattcgtgctagcgtaccaagcgattgggtagttggcgataaaactggtagttgcggtgcatacggtacggcaaatgattatgcggtagtctggccaaagaaccgagctcctcttataatttctgtatacactacaaaaaacgaaaaagaagccaagcatgaggataaagtaatcgcagaagcttcaagaatcgcaattgataaccttaaataaacttaactgtagccatcacttcattgtgatggcttaaatttcatcatgaaaacaatgaaaatcttcatctatttatcattagctatgagtagtttgtctacatatgctcagtgtgatttcatcaccgaaaattcgtcagtgatagatattaaaaacaacctagaatggcaggcttgcactttcggcatgcaatggaataaagagaatggatgtactggagaaccaactccccttactttgagtacagcttttggagaagcaaaaaaacaagggaatgggtggagactgccaaatatcgaggaattatccttactatcttcaaatagctgtcttaaaaatgaccttttcaaatcatattttgacataccccaagataatggtgaaggagcatcgccttattggagttcaacgcaaattcaaagcatacctt

SEQ ID NO: 207 - DNA, 879 bp, Enterobacter asburiae
atgtcacttaatgtaaaaccaagtagaatagccatcttgtttagctcttgtttagtttcaatatcatttttctcacaggccaatacaaagggaatcgatgagattaaaaaccttgaaacagatttcaatggtagaattggtgtctacgctttagacactggctcaggtaaatcattttcgtacaaagcaaatgaacgatttccattatgtagttctttcaaaggttttttagctgctgctgtattaaaaggctctcaagataatcaactaaatcttaatcagatcgtgaattataatacaagaagtttagagttctattcacccatcacaactaaatataaagataatggaatgtcattaggtgatatggctgctgccgctttacaatatagcgacaatggtgctactaatattattcttgaacgatatatcggtggtcctgagggtatgactaaattcatgcggtcgattggagataaagattttagactcgatcgttgggagttagatctaaacacagcaattcctggcgatgaacgtgacacatctacacctgcagcagtagctaagagcctgaaaacccttgctctgggtaacatacttaatgagcgtgaaaaggaaacctatcagacatggttaaagggtaacacaaccggtgcagcgcgtattcgtgctagcgtaccaagcgattgggtagttggcgataaaactggtagttgcggagcatacggtacggcaaatgattatgcggtagtctggccaaagaaccgagctcctcttataatttctgtatacactacaaaaaacgaaaaagaagccaagcatgaggataaagtaatcgcagaagcttcaagaatcgcaattgataaccttaaataa

SEQ ID NO: 208 - DNA, 879 bp, Enterobacter cloacae
atgtcacttaatgtaaaacaaagtagaatagccatcttgtttatctcttgtttattttcaatatcatttttctcacaggccaatacaaagggtatcgatgagattaaaaaccttgaaacagatttcaatggtagagttggtgtctacgctttagacactggctcaggtaaatcattttcgtacaaagcaaatgaacgatttccattatgtagttctttcaaaggctttttagctgctgctgtattaaaaggctctcaagataatcaactcaatcttaatcagatcgtgaattacaatacaagaagtttagagttccattcacccatcacaactaaatataaagataatggaatgtcattaggtgatatggctgctgccgctttacaatatagcgacaatggtgctactaatattattcttgaacgttatatcggtggtcctgagggtatgactaaattcatgcggtcgattggagatgaagattttagactcgatcgttgggagttagatctaaacacagctattccaggcgatgaacgtgacacatctacacctgcagcagtagctaagagcctgaaaactcttgctctgggtaacatacttagtgagcgtgaaaaggaaacctatcagacatggttaaagggtaacacaaccggtgcagcgcgtattcgtgctagcgtaccaagcgattgggtagttggcgataaaactggtagttgcggagcatacggtactgcaaatgattatgcggtagtctggccaaagaaccgggctcctcttataatttctgtatacacaacaaaaaacgaaaaagaagccaagcatgaggataaagtaatcgcagaagcttcaagaatygcaattgataaccttaaataa

SEQ ID NO: 209 - DNA, 879 bp, Enterobacter cloacae
atgtcacttaatgtaaaaccaagtagaatagccatcttgtttagctcttgtttagtttcaatatcatttttctcacaggccaatacaaagggcatcgatgagattaaagaccttgaaacagatttcaatggtagaattggtgtctacgctttagacactggctcaggcaaatcattttcatacaaagcaaatgaacgatttccattatgtagttctttcaaaggttttttagctgctgctgtattaaaaggctctcaagataatcaactaaatcttaatcagatcgtgaattataatacaagaagtttagagttccattcacccatcacaactaaatataaagataatggaatgtcattaggtgatatggctgctgcagctttacaatatagcgacaatggtgctactaatattatccttgaacgatatatcggtggtcctgagggtatgactaaattcatgcggtcgattggagataaagattttagactcgatcgttgggagttagatctaaacacagctattcctggcgatgaacgtgacacatctacacctgcagcagtagctaagagcctgaaaacccttgcactgggtaacatactcaatgagcgtgaaaaggaaacctatcagacatggttaaagggtaacacaaccggtgcagcgcgtattcgtgctagcgtaccaagcgattgggtagttggcgataaaactggtagttgcggtgcatacggtacggcaaatgattatgcggtagtctggccaaagaaccgagctcctcttataatttctgtatacactacaaaatacgaaaaagaagccaagcatgaggataaagtaatcgcagaagcttcaagaatcgcaattgataaccttaaataa

SEQ ID NO: 210 - DNA, 879 bp, Enterobacter cloacae
atgtcacttaatgtaaaaccaagtagaatagccatcttgtttagctcttgtttagtttcaatatcatttttctcacaggccaatacaaagggcatcgatgagattaaagaccttgaaaaagatttcaatggtagaattggtgtctacgctttagacactggctcaggcaaatcattttcatacaaagcaaatgaacgatttccattatgtagttctttcaaaggttttttagctgctgctgtattaaaaggctctcaagataatcaactaaatcttaatcagatcgtgaattataatacaagaagtttagagttccattcacccatcacaactaaatataaagataatggaatgtcattaggtgatatggctgctgcagctttacaatatagcgacaatggtgctactaatattatccttgaacgatatatcggtggtcctgagggtatgactaaattcatgcggtcgattggagataaagattttagactcgatcgttgggagttagatctaaacacagctattcctggcgatgaacgtgacacatctacacctgcagcagtagctaagagcctgaaaacccttgcactgggtaacatactcaatgagcgtgaaaaggaaacctatcagacatggttaaagggtaacacaaccggtgcagcgcgtattcgtgctagcgtaccaagcgattgggtagttggcgataaaactggtagttgcggtgcatacggtacggcaaatgattatgcggtagtctggccaaagaaccgagctcctcttataatttctgtttacactacaaaaaacgaaaaagaagccaagcatgaggataaagtaatcgcagaagcttcaagaatcgcaattgataaccttaaataa

SEQ ID NO: 211 - DNA, 879 bp, Enterobacter cloacae
atgtcaattaatgcaaagaaaagtaaagcagctgtctttttaagctattttttaatccctatatcattttattcacaggctgatacaaatgccatggatgagattaagaaactggaaacaggttttggtggcagggttggcgtctacgctttagacactggctctggtaaatcattttcatacagagcgaatgaacgatttcctctttgtagttcttttaaaggttttttagccgctgctgtattaaagggctcgcaggataatcaactgaatattaatgagattgtaaattataataaaagaagtttagaaccccactcccctattacgcaaaaatataaagaaaacggaatgtctctaggtgatatggctgctgccgctttacaatatagcgacaatggtgctgctaatattattcttgagcgttatatcggtggtcctgaaggtatgactaatttcatgcggtctattggagatgaagactttagactcgatcgttgggagttagatctaaatacagctattcctggcgatgaacgtgacacttcaacacccgcagctgtaggtaaaagtttaaaaaaccttgctctgggcaatatacttaacgatcatgaaaaggaaacatatcagacatggttaaagggtaatacaaccggcgcagcgcgtattcgtgctagcgtaccaagcgactgggtcgttggcgataaaaccggtacttgtggagcatacggtacggcaaatgattatgcggttgtctggccaaaaaacagggctcctcttatcatttctgtgtacactacaaaaagtgaaaaagaagcaaagcatgacgagaaggtaatcgaagaagcttcaagaattgcaattacacaccttaagtaa

(19) United States
(12) Patent Application Publication
(10) Pub. No.: US 2019/0352700 A1
(43) Pub. Date: Nov. 21, 2019

(54) DETECTION METHOD

(71) Applicant: THE SECRETARY OF STATE FOR HEALTH, London (GB)

(72) Inventors: John Mark SUTTON, Salisbury, Wiltshire (GB); Carrie TURNER, Salisbury, Wiltshire (GB); Collette ALLEN, Salisbury, Wiltshire (GB)

(21) Appl. No.: 16/096,917

(22) PCT Filed: Apr. 27, 2017
(86) PCT No.: PCT/GB2017/051178

§ 371 Date: Oct. 26, 2018

(30) Foreign Application Priority Data
Apr. 27, 2016 (GB) 1607356.1

(51) Int. Cl. C12Q 1/689

(52) U.S. Cl. CPC C12Q 1/689; C12Q 2600/158

(57) ABSTRACT

Carbapenem-resistant bacteria is detected and carbapenem-resistant bacteria infection is diagnosed. Primers and probes are used for particular carbapenemase genes, which enable the accurate detection of carbapenem-resistant bacteria.

### FIELD OF THE INVENTION

This invention relates to the detection of carbapenem-resistant bacteria and the diagnosis of carbapenem-resistant bacteria infection. More specifically, the invention relates to new primers and probes for particular carbapenemase genes, which enable the accurate detection of carbapenem-resistant bacteria.

### BACKGROUND OF THE INVENTION

Antibiotics are an important class of medicines. However, antibiotic resistance and the spread of resistant bacteria caused by the overuse and misuse of antibiotics is a major problem. The World Health Organisation describes the problems of antibiotic resistance as one of the biggest threats to human health today.

Resistance to beta-lactam antibiotics such as penicillin has come about through the emergence of bacterial strains which produce beta-lactamases; enzymes which hydrolyse the beta-lactam ring of beta-lactam antibiotics, ablating their antibacterial properties.

Carbapenems are a powerful group of broad spectrum beta-lactam (penicillin-related) antibiotics. Carbapenems have proved particularly useful as they have a structure that renders them resistant to most beta-lactamases, such that carbapenems are effective against bacteria which may be resistant to other beta-lactam antibiotics. Thus, in many cases carbapenems are the last line of defence against multi-resistant bacterial infections, particularly multi-resistant gram-negative bacteria.

Resistance has begun to emerge to carbapenems. Bacteria now exist which encode carbapenemases; a subset of beta-lactamases that are able to cleave the modified beta-lactam ring found in carbapenems, rendering them inactive. Carbapenem resistance is not due to a single carbapenemase enzyme, instead multiple carbapenemases have emerged.

Carbapenems are encoded on horizontally-transferable plasmids and have now spread globally.

An increase in antibiotic resistance, particularly carbapenemase resistance, in Gram-negative bacteria is especially concerning due to the speed with which resistance is emerging and because there are fewer new and developmental antibiotics against Gram-negative bacteria.

As well as a need for new antibiotics to treat carbapenem-resistant bacteria, there is also a need for screening methods to enable the detection of carbapenem-resistant bacteria. Early detection of infection typically allows for a more effective therapeutic treatment with a correspondingly more favourable clinical outcome. In view of the increasing threat and global prevalence carbapenem-resistant bacteria, new strategies are required for more effective prevention, treatment, and diagnosis of carbapenem-resistant bacteria infection. Ideally, diagnosis would be made by a technique that accurately, rapidly, and simultaneously detects a plurality of different carbapenemase enzymes at a single point in time, thereby minimizing progression of infection during the time required for diagnosis.

### SUMMARY OF THE INVENTION

Previous attempts to develop new diagnostic methods for carbapenem-resistant bacteria infection have typically focused on substrate-based methods. In more detail, samples have been plated on media containing a carbapenem antibiotic and assessed for bacterial growth. Bacteria able to grow in the presence of a carbapenem antibiotic are classed as carbapenem-resistant.

More recently, there have been attempts to develop nucleic acid-based methods for detecting carbapenem-resistant bacteria. Commercial kits for use in such methods have been developed. However, these methods and kits have been focused on the detection of individual carbapenemase families (such as KPC carbapenemases), and as such it is not possible to detect multiple carbapenemase using a single method with commercially available reagents.

Furthermore, as well as the existence of multiple different carbapenemase families, sequence variation within an individual carbapenemase family also makes it difficult to reliably identify the presence of carbapenem-resistant bacteria. Even if multiple assays are conducted for different carbapenemase families, variation within individual families may mean that some carbapenem-resistant bacteria are not detected, potentially resulting in false negative results.

The present inventors have conducted a detailed analysis of the sequences of the OXA-48-like, VIM, KPC, NDM, IMP and IMI carbapenemase genes. Based on the results of this analysis, the inventors have designed new primers and probes which maximize coverage of the different OXA-48-like, VIM, KPC, NDM, IMP and IMI carbapenemase gene variants. This reduces the risk of false negatives.

Another advantage provided by the present invention is that the mode of detection of the carbapenemase genes is flexible; it can be run using real-time/quantitative PCR (qPCR) standard PCR or techniques such as recombinase polymerase amplification (RPA) depending on the equipment available.

Therefore, the present invention allows for the flexible, accurate, rapid and sensitive detection of carbapenem-resistant bacteria and the diagnosis of carbapenem-resistant bacteria infection through a measurement of the KPC, OXA-48-like, NDM, IMP, VIM and/or IMI carbapenemase genes in a biological sample at a single point in time. Simultaneous detection of extended spectrum beta lactamases, such as CTX-M genes mediating resistance to third generation cephalosporins, may be usefully achieved from the same sample.

Accordingly, the present invention provides a method for determining the presence and/or amount of one or more carbapenemase-producing bacteria in a sample comprising determining the presence and/or amount of one or more carbapenemase gene selected from a KPC gene, an OXA-48-like gene, an NDM gene, an IMP gene, a VIM gene and/or an IMI gene in said sample, in which the presence and/or amount of the one or more carbapenemase gene is determined using at least one oligonucleotide specific for said one or more carbapenemase gene, wherein: (a) the one or more carbapenemase gene is a KPC gene and the at least one oligonucleotide specific for the KPC gene hybridises to a nucleic acid sequence within a target region of the KPC gene comprising bases 100 to 310 of the KPC gene; (b) the one or more carbapenemase gene is an OXA-48-like gene and the at least one oligonucleotide specific for the OXA-48-like gene hybridises to a nucleic acid sequence within a target region of the OXA-48-like gene comprising bases 120 to 360 of the OXA-48-like gene; (c) the one or more carbapenemase gene is an NDM gene and the at least one oligonucleotide specific for the NDM gene: (i) is at least 25 bases in length and hybridises to a nucleic acid sequence within a target region of the NDM gene comprising bases 250 to 480 of the NDM gene; and/or (ii) hybridises to a nucleic acid sequence within a target region of the NDM gene comprising bases 350 to 480 of the NDM gene; (d) the one or more carbapenemase gene is an IMP gene and the at least one oligonucleotide specific for the IMP gene: (i) hybridises to a nucleic acid sequence within a target region of the IMP gene comprising bases 730 to 783 of the IMP gene; (ii) hybridises to a nucleic acid sequence within a target region of the IMP gene comprising bases 850 to 886 of the IMP gene; and/or (iii) hybridises to a nucleic acid sequence within a target region of the IMP gene comprising bases 784 to 860 of the IMP gene; (e) the one or more carbapenemase gene is a VIM gene and the at least one oligonucleotide specific for the VIM gene: (i) hybridises to a nucleic acid sequence within a target region of the VIM gene comprising bases 120 to 195 of the VIM gene; (ii) comprises a region of at least 34 contiguous bases from SEQ ID NO: 25, or from a nucleic acid sequence having at least 95% sequence identity to the full-length of SEQ ID NO: 25, and which hybridises to a nucleic acid sequence within a target region of the VIM gene comprising bases 280 to 320 of the VIM gene; and/or (iii) is 30 to 60 bases in length and hybridises to a nucleic acid sequence within a target region of the VIM gene comprises bases 190 to 270 of the VIM gene; and/or (0 the one or more carbapenemase gene is an IMI gene and the at least one oligonucleotide specific for the IMI gene: (i) hybridises to a nucleic acid sequence within a target region of the IMI gene comprising bases 418 to 446 of the IMI gene; (ii) hybridises to a nucleic acid sequence within a target region of the IMI gene comprising bases 507 to 537 of the IMI gene or IMI gene comprising bases 517 to 545 of the IMI gene; and/or (iii) hybridises to a nucleic acid sequence within a target region of the IMP gene comprising bases 452 to 500 of the IMI gene.

In some embodiments, the at least one oligonucleotide sequence specific for the KPC gene: (a) hybridises to a nucleic acid sequence within a target region of the KPC gene comprising bases 100 to 180 of the KPC gene; (b) hybridises to a nucleic acid sequence within a target region of the KPC gene comprising bases 240 to 310 of the KPC gene; and/or (c) hybridises to a nucleic acid sequence within a target region of the KPC gene comprising bases 180 to 240 of the KPC gene; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the OXA-48-like gene: (a) hybridises to a nucleic acid sequence within a target region of the OXA-48-like gene comprising bases 120 to 180 of the OXA-48-like gene; (b) hybridises to a nucleic acid sequence within a target region of the OXA-48-like gene comprising bases 250 to 360 of the OXA-48-like gene; and/or (c) hybridises to a nucleic acid sequence within a target region of the OXA-48-like gene comprising bases 180 to 250 of the OXA-48-like gene; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the NDM gene: (a) is at least 25 bases in length and hybridises to a nucleic acid sequence within a target region of the NDM gene comprising bases 250 to 350, preferably 280 to 330 of the NDM gene; (b) hybridises to a nucleic acid sequence within a target region of the NDM gene comprising bases 400 to 450, preferably 410 to 450 of the NDM gene; and/or (c) hybridises to a nucleic acid sequence within a target region of the NDM gene comprising bases 350 to 410, preferably 360 to 410 of the NDM gene; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the IMP gene: (a) hybridises to a nucleic acid sequence within a target region of the IMP gene comprising bases 750 to 783 of the IMP gene; (b) comprises a nucleic acid sequence of any one of SEQ ID NOs: 12-22, or a nucleic acid sequence having at least 80% sequence identity to the full-length of any one of SEQ ID NOs: 12-22, and which hybridises to a nucleic acid sequence within a target region of the IMP gene comprising bases 850 to 886 of the IMP gene; and/or (c) is at least 30 bases in length and hybridises to a nucleic acid sequence within a target region of the IMP gene comprising bases 784 to 860 of the IMP gene; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the VIM gene: (a) hybridises to a nucleic acid sequence within a target region of the VIM gene comprising bases 130 to 180 of the VIM gene; (b) comprises a region of at least 34 contiguous bases from SEQ ID NO: 25, or from a nucleic acid sequence having at least 95% sequence identity to the full-length of SEQ ID NO: 25, and which hybridises to a nucleic acid sequence within a target region of the VIM gene comprising bases 283 to 318 of the VIM gene; and/or (c) is 30 to 60 bases in length and hybridises to a nucleic acid sequence within a target region of the VIM gene comprises bases 210 to 265 of the VIM gene; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the IMI gene: (a) hybridises to a nucleic acid sequence within a target region of the IMI gene comprising bases 418 to 446 of the IMI gene; (b) hybridises to a nucleic acid sequence within a target region of the IMI gene comprising bases 507 to 537 of the IMI gene, and/or comprises or consists of SEQ ID NO: 29 or 30, or a nucleic acid sequence having at least 80% sequence identity to the full-length of SEQ ID NO: 29 or 30, and hybridises to a nucleic acid sequence within a target region of the IMP gene comprising base 517 to 560, preferably 517 to 545, of the IMI gene; and/or (c) hybridises to a nucleic acid sequence within a target region of the IMI gene comprising bases 452 to 500 of the IMI gene, wherein optionally said oligonucleotide comprises a region of at least 40 contiguous bases from SEQ ID NO: 207, or from a nucleic acid sequence having at least 80% sequence identity to the full-length of SEQ ID NO: 207; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the KPC gene comprises a nucleic acid sequence selected from: (a) a nucleic acid sequence of SEQ ID NO: 1, or a nucleic acid sequence having at least 80% sequence identity to the full-length of the nucleic acid sequence of SEQ ID NO: 1; (b) a nucleic acid sequence of SEQ ID NO: 2, or a nucleic acid sequence having at least 80% sequence identity to the full-length of the nucleic acid sequence of SEQ ID NO: 2; and/or (c) a nucleic acid sequence of SEQ ID NO: 3, or a nucleic acid sequence having at least 80% sequence identity to the full-length of the nucleic acid sequence of SEQ ID NO: 3; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the OXA-48-like gene comprises a nucleic acid sequence selected from: (a) a nucleic acid sequence of SEQ ID NO: 4, or a nucleic acid sequence having at least 80% sequence identity to the full-length of the nucleic acid sequence of SEQ ID NO: 4; (b) a nucleic acid sequence of SEQ ID NO: 5, or a nucleic acid sequence having at least 80% sequence identity to the full-length of the nucleic acid sequence of SEQ ID NO: 5; and/or (c) a nucleic acid sequence of SEQ ID NO: 6, or a nucleic acid sequence having at least 80% sequence identity to the full-length of the nucleic acid sequence of SEQ ID NO: 6; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the NDM gene comprises a nucleic acid sequence selected from: (a) a nucleic acid sequence of SEQ ID NO: 7, or a nucleic acid sequence having at least 80% sequence identity to the full-length of the nucleic acid sequence of SEQ ID NO: 7; (b) a nucleic acid sequence of SEQ ID NO: 8, or a nucleic acid sequence having at least 80% sequence identity to the full-length of the nucleic acid sequence of SEQ ID NO: 8; and/or (c) a nucleic acid sequence of SEQ ID NO: 9, or a nucleic acid sequence having at least 80% sequence identity to the full-length of the nucleic acid sequence of SEQ ID NO: 9; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the IMP gene comprises a nucleic acid sequence selected from: (a) a nucleic acid sequence of SEQ ID NO: 10 or 11, or a nucleic acid sequence having at least 80% sequence identity to the full-length of the nucleic acid sequence of SEQ ID NO: 10 or 11; (b) a nucleic acid sequence of any one of SEQ ID NOs: 12-22, or a nucleic acid sequence having at least 80% sequence identity to the full-length of the nucleic acid sequence of any one of SEQ ID NOs: 12-22; and/or (c) a nucleic acid sequence of SEQ ID NO: 23, or a nucleic acid sequence having at least 80% sequence identity to the full-length of the nucleic acid sequence of SEQ ID NO: 23; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the VIM gene comprises a nucleic acid sequence selected from: (a) a nucleic acid sequence of SEQ ID NO: 24, or a nucleic acid sequence having at least 80% sequence identity to the full-length of the nucleic acid sequence of SEQ ID NO: 24; (b) a nucleic acid sequence of SEQ ID NO: 25, or a nucleic acid sequence having at least 95% sequence identity to the full-length of the nucleic acid sequence of SEQ ID NO: 25; and/or (c) a nucleic acid sequence of SEQ ID NO: 26 or 27, or a nucleic acid sequence having at least 80% sequence identity to the full-length of the nucleic acid sequence of SEQ ID NO: 26 or 27; wherein optionally the at least one oligonucleotide of (a) is a forward primer, the at least one oligonucleotide of (b) is a reverse primer; and/or the at least one oligonucleotide of (c) is a probe.

In some embodiments, the at least one oligonucleotide sequence specific for the IMI gene comprises a nucleic acid sequence selected from: (a) a nucleic acid sequence of SEQ ID NO: 28, or a nucleic acid sequence having at least 80% sequence identity to the full-length of the nucleic acid sequence of SEQ ID NO: 28; (b) a nucleic acid sequence of SEQ ID NO: 29 or 30, or a nucleic acid sequence having at least 80% sequence identity to the full-length of the nucleic acid sequence of SEQ ID NO: 29 or 30; and/or (c) a nucleic acid sequence of SEQ ID NO: 31, or a nucleic acid sequence having at least 80% sequence identity to the full-length of the nucleic acid sequence of SEQ ID NO: 31; wherein optionally the at least one oligonucleotide of (a) is a forward primer, the at least one oligonucleotide of (b) is a reverse primer; and/or the at least one oligonucleotide of (c) is a probe.

The presence and/or amount of the KPC gene, OXA-48-like gene, NDM gene, IMP gene, VIM gene, and/or IMI gene may be determined using: (a) the corresponding forward primer sequences of the invention; (b) the corresponding reverse primer sequences of the invention; (c) the corresponding forward and reverse primer sequences of the invention; (d) the corresponding forward primer, reverse primer and probe sequences of the invention.

The presence and/or amount of any one, any two, any three, any four, any five or all six of a KPC gene, an OXA-48-like gene, an NDM gene, an IMP gene, a VIM gene and/or an IMI gene may be determined; and optionally the presence and/or amount of (i) a KPC gene, an OXA-48-like gene, an NDM gene, an IMP gene and a VIM gene; or (ii) a KPC gene, an OXA-48-like gene, an NDM gene, an IMP gene, a VIM gene and an IMI gene; is determined.

The method of the invention may further comprise determining: (a) the presence and/or amount of one or more additional carbapenemase gene; and/or (b) the presence and/or amount of one or more CTX-M β-lactamase. In some embodiments, the one or more additional carbapenemase gene is selected from an SME gene, a GES gene and an SPM gene. The presence and/or amount of the one or more additional carbapenemase gene may be determined using at least one oligonucleotide specific for said one or more additional carbapenemase gene, and/or the presence and/or amount of the one or more CTX-M β-lactamase may be determined using at least one oligonucleotide specific for CTX-M β-lactamase. The at least one oligonucleotide specific for CTX-M β-lactamase may be selected from: (a) a nucleic acid sequence of SEQ ID NO: 32 or 33, or a nucleic acid sequence having at least 80% sequence identity to the full-length of the nucleic acid sequence of SEQ ID NO: 32 or 33; (b) a nucleic acid sequence of SEQ ID NO: 34 or 35, or a nucleic acid sequence having at least 80% sequence identity to the full-length of the nucleic acid sequence of SEQ ID NO: 34 or 35; and/or (c) a nucleic acid sequence of SEQ ID NO: 36 or 37, or a nucleic acid sequence having at least 80% sequence identity to the full-length of the nucleic acid sequence of SEQ ID NO: 36 or 37; wherein optionally the at least one oligonucleotide of (a) is a forward primer, the at least one oligonucleotide of (b) is a reverse primer; and/or the at least one oligonucleotide of (c) is a probe.

The presence and/or amount of the KPC gene, the OXA-48-like gene, the NDM gene, the IMP gene, the VIM gene, the IMI gene and/or the one or more additional carbapenemase gene and/or the one or more CTX-M β-lactamase may be determined by PCR and/or hybridisation. In some embodiments, the presence and/or amount of the KPC gene, the OXA-48-like gene, the NDM gene, the IMP gene, the VIM gene, the IMI gene and/or the one or more additional carbapenemase gene and/or the one or more CTX-M β-lactamase is determined by Recombinase Polymerase Amplification (RPA) or real-time PCR.

According to the present invention, (a) the presence and/or amount the KPC gene is compared with the presence and/or amount of the KPC gene in a control sample; (b) the presence and/or amount the OXA-48-like gene is compared with the presence and/or amount of the OXA-48-like in a control sample; (c) the presence and/or amount the NDM gene is compared with the presence and/or amount of the NDM gene in a control sample; (d) the presence and/or amount the IMP gene is compared with the presence and/or amount of the IMP gene in a control sample; (e) the presence and/or amount the VIM gene is compared with the presence and/or amount of the VIM gene in a control sample; (f) the presence and/or amount the IMI gene is compared with the presence and/or amount of the IMI gene in a control sample; (g) the presence and/or amount the one or more additional carbapenemase gene is compared with the presence and/or amount of the one or more additional carbapenemase gene in a control sample; and/or (h) the presence and/or amount the one or more CTX-M β-lactamase is compared with the presence and/or amount of the one or more CTX-M β-lactamase in a control sample.

A method for diagnosing a carbapenem-resistant bacteria infection in an individual according to the invention may comprise comprising carrying out said method on a sample obtained from the individual. Said sample may be a sample of whole blood, serum, plasma, cerebral spinal fluid, saliva, urine, cells, a cellular extract, a stool sample, a tissue sample or a tissue biopsy, or a swab from an individual, such as a rectal swab, or a swab from the environment.

Said invention further provides a device for use in the method of the invention, which comprises one or more oligonucleotide specific for the KPC gene, the OXA-48-like gene, the NDM gene, the IMP gene, the VIM gene, the IMI gene and/or the one or more additional carbapenemase gene. The one or more oligonucleotide specific for the KPC gene, the OXA-48-like gene, the NDM gene, the IMP gene, the VIM gene, the IMI gene and/or the one or more additional carbapenemase gene may be an oligonucleotide of the invention. In some embodiments, the one or more oligonucleotide may be immobilised on a surface.

### DETAILED DESCRIPTION OF THE INVENTION

The present invention allows for the flexible, accurate, rapid and sensitive detection of carbapenem-resistant bacteria and the diagnosis of carbapenem-resistant bacteria infection through a measurement of the KPC, OXA-48-like, NDM, IMP, VIM and/or IMI carbapenemase genes in a biological sample at a single time point (“snapshot”) or during the course of an infection.

### Carbapenemases and Carbapenem-Resistant Bacteria

Carbapenems are a critically important sub-class of beta-lactam antibiotics that possess the broadest spectrum of activity and highest activity against Gram-positive and Gram-negative bacteria, which means they are often used as a last line of defence in treating persistent infections or infections with bacteria known or suspected of being resistant to other antibiotics.

Structurally, carbapenems are very similar to penicillins, but the sulphur atom at position 1 of the beta-lactam ring has been replaced with an unsubstituted carbon atom. Resistance to carbapenem antibiotics is emerging in both Gram-negative and Gram-positive bacteria. Examples of carbapenem-resistant Gram-negative bacteria include Pseudomonas spp., Acinetobacter spp. and Stenotrophomonas spp., as well as the Enterobacteriaceae (for example Klebsiella spp., Escherichia coli and Enterobacter spp.). Examples of carbapenem-resistant Gram-positive bacteria include Staphylococcus spp., Streptococcus spp., Enterococcus spp. and Nocardia spp.

Mechanisms of resistance to carbapenems include the production of carbapenemases, efflux pumps and mutations that alter the expression and/or function of porins and penicillin binding proteins. Carbapenemases are specific beta-lactamases with the ability to hydrolyse carbapenems. Production of carbapenemases is a common cause of carbapenem resistance. Bacteria which produce one or more carbapenemase are referred to as carbapenemase-producing bacteria. As such bacteria are either (entirely) resistant to, or less susceptible to, carbapenem antibiotics than bacteria which do not possess a mechanism of carbapenem resistance, they are also referred to herein as carbapenem-resistant bacteria. Herein the terms carbapenemase-producing bacteria and carbapenem-resistant bacteria are used interchangeably. The present invention may be used in the detection and/or diagnosis of both Gram-positive and/or Gram-negative carbapenem-resistant bacteria, including any of the bacterial species identified herein.

The production of carbapenemases is particularly associated with Gram-negative carbapenem-resistant bacteria. Typically, therefore, the present invention relates to the detection and/or diagnosis of Gram-negative carbapenemase-producing bacteria (also referred to herein as Gram-negative carbapenem-resistant bacteria), for example Pseudomonas spp., Acinetobacter spp. and Stenotrophomonas spp., and/or the Enterobacteriaceae. In a preferred embodiment, the present invention relates to the detection and/or diagnosis of carbapenem-resistant Enterobacteriaceae, such as Klebsiella spp., Escherichia coli, Citrobacter spp., Cronobacter spp., Leclercia spp., Shigella spp., Salmonella spp. and Enterobacter spp.

The present invention may be used to detect and/or diagnose at least one species of carbapenem-resistant bacteria, such as those identified herein, particularly at least one species of Gram-negative carbapenem-resistant bacteria. For example, the present invention may be used to detect at least one, at least two, at least three, at least four, at least five, at least six, at least seven, at least eight, at least nine, at least ten or more species of carbapenemase-producing bacteria (carbapenem-resistant bacteria), particularly Gram-negative carbapenem-resistant bacteria.

Beta-lactamases are grouped into four distinct categories (A to D) based on their structure. Class B beta-lactamases are Zn²⁺-dependent and are all carbapenemases. Class A, C and D beta-lactamases use serine as a nucleophile to hydrolyse the beta-lactam bond.

The present invention relates to the detection or diagnosis of carbapenemase-producing bacteria (carbapenem-resistant bacteria) by detecting the presence and/or amount of: (i) any one, two or all three of the class B carbapenemases, VIM carbapenemases and/or NDM carbapenemases and/or IMP carbapenemases; and/or the class A carbapenemase KPC carbapenemases and/or IMI carbapenemases; and/or the class D carbapenemases, OXA-48-like carbapenemases. The invention relates to the detection of any one of these carbapenemases by any of the means described herein. Thus, the present invention relates to the detection of one or more KPC gene, one or more OXA-48-like gene, one or more NDM gene, one or more IMP gene, one or more VIM gene and/or one or more IMI gene. Alternatively, any combination of these carbapenemases may be detected according to the present invention. For example, the invention may relate to the detection of any two (for example: VIM and NDM; VIM and IMP; VIM and KPC; VIM and OXA-48-like; VIM and IMI; NDM and IMP; NDM and KPC; NDM and OXA-48-like; NDM and IMI; IMP and KPC; IMP and OXA-48-like; IMP and IMI; KPC and OXA-48-like; KPC and IMI; or OXA-48-like and IMI), any three (for example: VIM, NDM and IMP; VIM, NDM and KPC; VIM, NDM and OXA-48-like; VIM, NDM and IMI; VIM, IMP and KPC; VIM, IMP and OXA-48-like; VIM, IMP and IMI; VIM, KPC and OXA-48-like; VIM, KPC and IMI; VIM, OXA-48-lie and IMI; NDM, IMP and KPC; NDM, IMP and OXA-48-like; NDM, IMP and IMI; NDM, KPC and OXA-48-like; NDM, KPC and IMI; NDM, OXA-48-like and IMI; IMP, KPC and OXA-48-like; IMP, KPC and IMI; IMP, OXA-48-like and IMI; or KPC, OXA-48-like and IMI), any four (for example: VIM, NDM, IMP and KPC; VIM, NDM, IMP and OXA-48-like; VIM, NDM, IMP and IMI; VIM, IMP, KPC and OXA-48-like; VIM, NDM, KPC and OXA-48-like; or NDM, IMP, KPC and OXA-48-like; VIM, NDM, OXA-48-like and IMI; VIM, IMP, OXA-48-like and IMI; VIM, KPC, OXA-48-like and IMI; NDM, IMP, OXA-48-like and IMI; NDM, KPC, OXA-48-like and IMI; IMP, KPC, OXA-48-like and IMI; VIM, IMP, KPC and IMI; VIM, NDM, KPC and IMI; or NDM, IMP, KPC and IMI), any five of the carbapenemase genes (for example: VIM, NDM, IMP, KPC and OXA-48-like; VIM, NDM, IMI, KPC and OXA-48-like; IMI, NDM, IMP, KPC and OXA-48-like; VIM, NDM, IMP, KPC and IMI; VIM, IMP, KPC, OXA-48-like and IMI; or VIM, NDM, IMP, OXA-48-like and IMI), or all six of the carbapenemase genes (VIM, NDM, IMP, KPC, IMI and OXA-48-like). In a preferred embodiment, the invention relates to the detection of one or more KPC gene, one or more OXA-48-like gene, one or more NDM gene, one or more IMP gene and one or more VIM gene. In another preferred embodiment, the invention relates to the detection of one or more KPC gene, one or more OXA-48-like gene, one or more NDM gene, one or more IMP gene, one or more VIM gene and one or more IMI gene.

According to the present invention, disclosure of the detection of a given carbapenemase gene encompasses both the detection of the presence and/or amount of one or more member of said carbapenemase gene family in a sample, for example the presence and/or amount of at least two, at least three, at least four, at least five, at least six, at least seven, at least eight, at least nine, at least ten or more members of said carbapenemase gene family may be determined. As a non-limiting example, detection of a VIM carbapenemase gene encompasses the detection of at least one, at least two, at least three, at least four, at least five or more VIM genes, such as VIM-1, VIM-2 and/or VIM-7.

In a preferred embodiment, the presence and/or amount of any five of the carbapenemase genes (for example: VIM, NDM, IMP, KPC and OXA-48-like; VIM, NDM, IMI, KPC and OXA-48-like; IMI, NDM, IMP, KPC and OXA-48-like; VIM, NDM, IMP, KPC and IMI; VIM, IMP, KPC, OXA-48-like and IMI; or VIM, NDM, IMP, OXA-48-like and IMI), or all six of the carbapenemase genes (VIM, NDM, IMP, KPC, IMI and OXA-48-like) is determined.

The term “OXA-48-like carbapenemase” refers to a subgroup of OXA carbapenemases having a significant degree of sequence identity (at the amino acid and/or nucleic acid level) with OXA-48. An OXA-48-like carbapenemase may be defined as an OXA carbapenemase having at least 85%, at least 90%, at least 95%, at least 96%, at least 97%, at least 98%, at least 99% or more sequence identity (at the amino acid or nucleic acid level) with any OXA carbapenemase accession number or SEQ ID NO disclosed herein, or to any known OXA-48-like carbapenemase. Typically, an OXA-48-like carbapenemase according to the present invention may be defined as an OXA carbapenemase having at least 85%, at least 90%, at least 95%, at least 96%, at least 97%, at least 98%, at least 99% or more sequence identity with one or more of the following OXA carbapenemases (at the amino acid or nucleic acid level: OXA-181 (Accession No. GI 304368222, HM992946.1), OXA-232 GI 444236140, JX423831.1, 2677-3474), OXA-247 (GI 442769982, JX893517.1, 1-786), OXA-163 (GI 323817046, HQ700343.1, 1-786), OXA-204 (GI 408795221, JQ809466.1, 5375-6172), OXA-370 (GI 573006828, KF900153.1, 1-798), OXA-245 (GI 442577759, JX438001.1, 1-798), OXA-162 (GI 270312218, GU197550.1, 1-798), OXA-48 (GI AY236073.2, 2188-2985) and/or OXA-244 (GI 442577757, JX438000.1, 1-798). In a preferred embodiment, an OXA-48-like carbapenemase has at least 85%, at least 90%, at least 95%, at least 96%, at least 97%, at least 98%, at least 99% or more sequence identity with the OXA-48 carbapenemase (GI AY236073.2, 2188-2985). Additionally, one or more specific OXA-48-like genes for detection may be chosen on the basis of local or national prevalence data.

The term “VIM carbapenemase” includes all subgroups and subtypes of the VIM carbapenemase family, including but not limited to VIM-1, VIM-2 and/or VIM-7 carbapenemases, such as those identified by accession number and/or SEQ ID NO herein. Additionally, one or more specific VIM subtypes for detection may be chosen on the basis of local or national prevalence data. The term “VIM carbapenemase” also encompasses any carbapenemase having at least 85%, at least 90%, at least 95%, at least 96%, at least 97%, at least 98%, at least 99% or more sequence identity (at the amino acid or nucleic acid level) with any VIM carbapenemase accession number or SEQ ID NO disclosed herein, or to any known VIM carbapenemase.

The term “KPC carbapenemase” includes all subgroups and subtypes of the KPC carbapenemase family, including but not limited to KPC-1, KPC-2 and/or KPC-3, such as those identified by accession number and/or SEQ ID NO herein, or a variant having at least 85%, at least 90%, at least 95%, at least 96%, at least 97%, at least 98%, at least 99% or more sequence identity (at the amino acid or nucleic acid level) with any KPC carbapenemase accession number or SEQ ID NO disclosed herein, or to any known KPC carbapenemase. Additionally, one or more specific KPC subtypes for detection may be chosen on the basis of local or national prevalence data. This applies equally to other carbapenemase gene families disclosed herein. As a non-limiting example of the invention, the presence and/or amount of an OXA-48-like carbapenemase and the presence and/or amount of an KPC carbapenemase, such as KPC-1 and/or KPC-2, may be determined. As a non-limiting example, the presence and/or amount of OXA-48 may be determined and the presence and/or amount of KPC-2 may be determined. Any oligonucleotide specific for an OXA-48-like gene of the invention may be used in the detection of said OXA-48-like gene. In addition, the presence and/or amount of one or more VIM gene, one or more NDM gene, one or more IMP gene and/or one or more KPC gene and/or one or more IMI gene may also be determined.

The term “NDM carbapenemase” includes all subgroups and subtypes of the NDM carbapenemase family, including but not limited to NDM-1 carbapenemases, NDM-2 carbapenemases, NDM-3 carbapenemases, NDM-4 carbapenemases and NDM-5 carbapenemases, such as those identified by accession number and/or SEQ ID NO herein, or a variant having at least 85%, at least 90%, at least 95%, at least 96%, at least 97%, at least 98%, at least 99% or more sequence identity (at the amino acid or nucleic acid level) with any NDM carbapenemase accession number or SEQ ID NO disclosed herein, or to any known NDM carbapenemase. Additionally, one or more specific NDM subtypes for detection may be chosen on the basis of local or national prevalence data. As a non-limiting example, the presence and/or amount of an NDM carbapenemase, such as NDM-1, NDM-4 and/or NDM-5, is determined, either alone or in combination with the presence and/or amount of one or more OXA-48-like gene, one or more VIM gene, one or more IMP gene and/or one or more KPC gene, or any combination thereof as disclosed herein. As a non-limiting example, in some embodiments, the presence and/or amount of OXA-48 may be determined and the presence and/or amount of NDM-1, NDM-4 and/or NDM-5 may be determined. As another non-limiting example, in some embodiments, the presence and/or amount of OXA-181 may be determined and the presence and/or amount of NDM-1, NDM-4 and/or NDM-5 may be determined. As another non-limiting example, in some embodiments the presence and/or amount of OXA-232 may be determined and the presence and/or amount of NDM-1, NDM-4 and/or NDM-5 may be determined. Any oligonucleotide specific for an OXA-48-like gene of the invention may be used in the detection of said OXA-48, OXA-181 and/or OXA-232 gene. In addition, the presence and/or amount of one or more VIM gene, one or more IMP gene, one or more IMI gene and/or one or more KPC gene may also be determined.

The term “IMP carbapenemase” includes all subgroups and subtypes of the IMP carbapenemase family, including but not limited to IMP-1 and/or IMP-2 carbapenemases, such as those identified by accession number and/or SEQ ID NO herein. Additionally, one or more specific IMP subtypes for detection may be chosen on the basis of local or national prevalence data. The term “IMP carbapenemase” also encompasses any carbapenemase having at least 85%, at least 90%, at least 95%, at least 96%, at least 97%, at least 98%, at least 99% or more sequence identity (at the amino acid or nucleic acid level) with any IMP carbapenemase accession number or SEQ ID NO disclosed herein, or to any known IMP carbapenemase.

The term “IMI carbapenemase” includes all subgroups and subtypes of the IMI carbapenemase family, including but not limited to IMI-1 and/or IMI-2 carbapenemases, such as those identified by accession number and/or SEQ ID NO herein. Additionally, one or more specific IMI subtypes for detection may be chosen on the basis of local or national prevalence data. The term “IMI carbapenemase” also encompasses any carbapenemase having at least 85%, at least 90%, at least 95%, at least 96%, at least 97%, at least 98%, at least 99% or more sequence identity (at the amino acid or nucleic acid level) with any IMI carbapenemase accession number or SEQ ID NO disclosed herein, or to any known IMI carbapenemase.

As well as determining the presence and/or amount of one or more OXA-48-like gene, one or more VIM gene, one or more IMI gene, one or more NDM gene, one or more IMP gene and/or one or more KPC gene, the presence and/or amount of one or more additional carbapenemase may be determined. For example the presence and/or amount of at least two, at least three, at least four, at least five, at least six, at least seven, at least eight, at least nine, at least ten or more additional carbapenemase may determined in combination with determining the presence and/or amount of the one or more OXA-48-like, one or more IMI gene, one or more VIM, one or more NDM, one or more IMP and/or one or more KPC carbapenemase.

The one or more additional carbapenemase is not limited, and may be a class A, B, C or D carbapenemase. For example, the one or more additional carbapenemase may be selected from GES (class A), SME (class A), NmcA (class A), CphA (class B), CMY (class C), oxacillinase (OXA, class D), or others (e.g. SME, NMC, SPM, AIM, GIM, DIM and CcrA). Any one or any combination of these additional carbapenemase may be used according to the present invention, and in combination with any of the one or more OXA-48-like, one or more VIM, one or more NDM, one or more IMP and/or one or more KPC carbapenemases, or combinations thereof as disclosed herein. Any member of these carbapenemase families may be used in the context of the present invention. Non-exhaustive examples of OXA carbapenemases that may be used as a one or more additional carbapenemase include OXA-10, OXA-23, OXA-24/40, OXA-48, OXA-51, OXA-55, OXA-58, OXA-143, OXA-181 and OXA-232.

Thus, the present invention relates to a method for detecting the presence and/or amount of one or more carbapenemase-producing bacteria (carbapenem-resistant bacteria) comprising determining the presence and/or amount of one or more OXA-48-like gene, one or more VIM gene, one or more IMI gene, one or more NDM gene, one or more IMP gene or one or more KPC gene, or of any two (for example: VIM and NDM; VIM and IMP; VIM and KPC; VIM and OXA-48-like; VIM and IMI; NDM and IMP; NDM and KPC; NDM and OXA-48-like; NDM and IMI; IMP and KPC; IMP and OXA-48-like; IMP and IMI; KPC and OXA-48-like; KPC and IMI; or OXA-48-like and IMI), any three (for example: VIM, NDM and IMP; VIM, NDM and KPC; VIM, NDM and OXA-48-like; VIM, NDM and IMI; VIM, IMP and KPC; VIM, IMP and OXA-48-like; VIM, IMP and IMI; VIM, KPC and OXA-48-like; VIM, KPC and IMI; VIM, OXA-48-lie and IMI; NDM, IMP and KPC; NDM, IMP and OXA-48-like; NDM, IMP and IMI; NDM, KPC and OXA-48-like; NDM, KPC and IMI; NDM, OXA-48-like and IMI; IMP, KPC and OXA-48-like; IMP, KPC and IMI; IMP, OXA-48-like and IMI; or KPC, OXA-48-like and IMI), any four (for example: VIM, NDM, IMP and KPC; VIM, NDM, IMP and OXA-48-like; VIM, NDM, IMP and IMI; VIM, IMP, KPC and OXA-48-like; VIM, NDM, KPC and OXA-48-like; or NDM, IMP, KPC and OXA-48-like; VIM, NDM, OXA-48-like and IMI; VIM, IMP, OXA-48-like and IMI; VIM, KPC, OXA-48-like and IMI; NDM, IMP, OXA-48-like and IMI; NDM, KPC, OXA-48-like and IMI; IMP, KPC, OXA-48-like and IMI; VIM, IMP, KPC and IMI; VIM, NDM, KPC and IMI; or NDM, IMP, KPC and IMI), any five of the carbapenemase genes (for example: VIM, NDM, IMP, KPC and OXA-48-like; VIM, NDM, IMI, KPC and OXA-48-like; IMI, NDM, IMP, KPC and OXA-48-like; VIM, NDM, IMP, KPC and IMI; VIM, IMP, KPC, OXA-48-like and IMI; or VIM, NDM, IMP, OXA-48-like and IMI), or all six of the carbapenemase genes (VIM, NDM, IMP, KPC, IMI and OXA-48-like), wherein the presence and/or amount of at least one of a GES carbapenemase, an SME carbapenemase, an NmcA carbapenemase, a CphA carbapenemase, a CMY carbapenemase, an OXA carbapenemase, an IMI carbapenemase, a SPM carbapenemase, an AIM carbapenemase, a GIM carbapenemase, a DIM carbapenemase and/or a CcrA carbapenemase is also determined.

Said method may involve the detection of at least two, at least three, at least four, at least five, at least six, at least seven, at least eight, at least nine, at least ten, at least 11, or all 12 of an a GES carbapenemase, an SME carbapenemase, an NmcA carbapenemase, a CphA carbapenemase, a CMY carbapenemase, an OXA carbapenemase, a SPM carbapenemase, an AIM carbapenemase, a GIM carbapenemase, a DIM carbapenemase and/or a CcrA carbapenemase or any combination thereof (in addition to any of the combinations of an OXA-48-like carbapenemase, a VIM carbapenemase, an IMI carbapenemase, a KPC carbapenemase, an IMP carbapenemase and a NDM carbapenemase). As an example, said method may involve the detection of an SME carbapenemase (in addition to any one or more OXA-48-like gene, one or more VIM gene, one or more NDM gene, one or more IMP gene or one or more KPC gene, or any combination thereof as disclosed herein). The choice of particular carbapenemases to be used for screening may reflect local prevalence in clinical isolates and/or be directed by initial data on the infectious organism (for example; the detection of SME carbapenemase in relation to infections caused by Serratia marcesens).

The invention may involve determining the presence and/or amount of one or more CTX-M β-lactamase in addition to the detection of any combination of carbapenemase and/or additional carbapenemase genes as described herein. Examples of CTX-M β-lactamase that may be detected according to the present invention include, but are not limited to CTX-M14 and/or CTX-M15. Any suitable oligonucleotides may be used for determining the presence and/or amount of the one or more CTX-M β-lactamase, such as those disclosed herein.

In embodiments of the invention wherein the presence and/or amount of both an NDM carbapenemase gene and/or a KPC carbapenemase gene is determined, the KPC gene may preferably be a KPC-2 gene and the NDM gene may preferably be an NDM-1 gene, an NDM-4 gene and/or an NDM-5 gene, particularly preferably an NDM-1 gene.

The carbapenemase genes as described herein may have a nucleic acid sequence as shown in the sequences in the Sequence Information section herein. The relevant sequence identifiers are also shown. The one or more carbapenemase gene of the invention may have a sequence identity of at least 80% with the corresponding nucleic acid sequence shown in the Sequence Information section. Sequence identity may be calculated as described herein. A sequence identity of at least 80% includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO and/or to each and every accession number presented herein). As a non-limiting example, the OXA-48 gene may have at least 80% sequence identity with any of the OXA gene accession numbers or SEQ ID NO disclosed herein. This definition of sequence identity applies equally to the oligonucleotides of the invention with respect to the corresponding carbapenemase gene.

### Detection and Quantification of Carbapenem-Resistant Bacteria

The present inventors have developed new primer/probe sequences for OXA-48-like carbapenemase genes, VIM carbapenemase genes, NDM carbapenemase genes, IMP carbapenemase genes, IMI carbapenemase genes and KPC carbapenemase genes. These primers/probes have been designed to maximise coverage of different variants of the OXA-48-like, VIM, NDM, IMP, IMI and KPC carbapenemase genes respectively. Thus, the present invention provides primers/probes that enable the detection of KPC carbapenemase, OXA-48-like carbapenemase, NDM carbapenemase, IMP carbapenemase, VIM carbapenemase and/or IMI carbapenemase-producing bacteria with improved sensitivity, because they detect more KPC, OXA-48-like, NDM, IMP, VIM and IMI carbapenemase variants, and so the likelihood of false negatives is reduced.

### Decision Trees

A “decision rule” or a “decision tree” is a method used to classify individuals. This rule can take on one or more forms that are known in the art, as exemplified in Hastie et al., in “The Elements of Statistical Learning” Springer-Nerlag (Springer, New York (2001)). Analysis of biomarkers in the complex mixture of molecules within the sample generates features in a data set. A decision rule or a decision tree may be used to act on a data set of features to detect one or more carbapenemase-producing bacteria and/or diagnose a carbapenem-resistant bacterial infection.

The application of the decision rule or the decision tree does not require perfect classification. A classification may be made with at least about 60%, at least about 70%, at least about 80%, at least about 90% certainty or even more. The useful degree of certainty may vary, depending on the particular method of the present invention. “Certainty” is defined as the total number of accurately classified individuals divided by the total number of individuals subjected to classification. As used herein, “certainty” means “accuracy”.

Classification may also be characterized by its “sensitivity”. The “sensitivity” of classification relates to the percentage of samples containing carbapenemase-producing bacteria and/or carbapenem-resistant bacterial infections that were correctly identified. “Sensitivity” is defined in the art as the number of true positives divided by the sum of true positives and false negatives.

The “specificity” of a method is defined as the percentage of samples that were correctively identified as not having carbapenemase-producing bacteria and/or carbapenem-resistant bacterial infections compared with an uninfected/negative control(s). That is, “specificity” relates to the number of true negatives divided by the sum of true negatives and false positives.

Typically, the accuracy, sensitivity and/or specificity is at least about 90%, at least about 80%, at least about 70% or at least about 60%.

Diagnosing a carbapenem-resistant bacterial infection in an individual means to identify or detect the presence and/or amount of one or more carbapenemase-producing bacteria in the individual. This is achieved by determining the presence and/or amount of one more carbapenemase gene as described herein.

Because of the sensitivity of the present invention to detect a carbapenem-resistant bacterial infection before an overtly observable clinical manifestation, the diagnosis, identification or detection of a carbapenem-resistant bacterial infection includes the detection of the onset of a carbapenem-resistant bacterial infection, as defined above.

According to the present invention, a carbapenem-resistant bacterial infection may be diagnosed or detected, by determining the presence and/or amount of one or more carbapenemase-producing bacteria in a sample obtained from an individual. As used herein, “obtain” means “to come into possession of”. The present invention is particularly useful in predicting and diagnosing a carbapenem-resistant bacterial infection in an individual, who is suspected of having a carbapenem-resistant bacterial infection, or who is at risk of a carbapenem-resistant bacterial infection. The present invention may be used to confirm a clinical suspicion of a carbapenem-resistant bacterial infection.

### Controls or Reference Populations

The presence and/or amount of the one or more carbapenemase-producing bacteria in a sample may be measured relative to a control or reference population, for example relative to the corresponding carbapenemase-producing bacteria of a control or reference population. Herein the terms “control” and “reference population” are used interchangeably. The actual amount of the one or more carbapenemase produced by the one or more carbapenemase-producing bacteria, such as the mass, molar amount, concentration or molarity of the one or more carbapenemase may be assessed and compared with the corresponding value from the control or reference population. Alternatively, the amount of one or more carbapenemase may be compared with that of the control or reference population without quantifying the mass, molar amount, concentration or molarity of the one or more carbapenemase.

The control or reference population can be generated from one individual or a population of two or more individuals. The control or reference population, for example, may comprise three, four, five, ten, 15, 20, 30, 40, 50 or more individuals. Furthermore, the control or reference population and the individual's (test) sample that are compared in the methods of the present invention may be generated from the same individual, provided that the test and reference samples are taken at different time points and compared to one another. For example, a sample may be obtained from an individual at the start of a study period. A control or reference taken from that sample may then be compared to subsequent samples from the same individual. Such a comparison may be used, for example, to determine the progression of a carbapenemase-producing bacteria and/or carbapenem-resistant bacterial infection in the individual by repeated classifications over time.

The control or reference may be obtained, for example, from a population of carbapenemase-producing bacteria negative individuals (i.e. individuals negative for infection by one or more carbapenem-resistant bacteria) or carbapenemase-producing bacteria positive individuals (i.e. individuals positive for infection by one or more carbapenem-resistant bacteria).

Typically the control or reference population does not comprise one or more carbapenemase-producing bacteria and/or is not infected with one or more carbapenem-resistant bacteria (i.e. is negative for carbapenem-resistant bacterial infection). Alternatively, the control or reference population may comprise one or more carbapenemase-producing bacteria and/or be infected with one or more carbapenem-resistant bacteria (i.e. is positive for carbapenem-resistant bacterial infection) and may be subsequently diagnosed with a carbapenem-resistant bacterial infection using conventional techniques. For example, a population of carbapenem-resistant bacterial infection-positive individuals used to generate the reference or control may be diagnosed with carbapenem-resistant bacterial infection about 24, 48, 72, 96 or more hours after biological samples were taken from them for the purposes of generating a reference or control. In one embodiment, the population of carbapenem-resistant bacterial infection-positive individuals is diagnosed with carbapenem-resistant bacterial infection using conventional techniques about 0-36 hours, about 36-60 hours, about 60-84 hours, or about 84-108 hours after the biological samples were taken.

As described herein, the present invention relates to a method for determining the presence and/or amount of one or more carbapenemase-producing bacteria and/or diagnosing a carbapenem-resistant bacterial infection. Thus, in some instances it is sufficient to detect the presence of one or more carbapenemase-producing bacteria (i.e. carbapenem-resistant bacteria) in a sample. In such cases, the control or reference population may be positive or negative for carbapenemase-producing bacteria/carbapenem-resistant bacterial infection.

In other instances, the amount of the one or more carbapenemase-producing bacteria (i.e. carbapenem-resistant bacteria) is determined relative to a control or reference population. In such cases, the amount of the one or more carbapenemase-producing bacteria (i.e. carbapenem-resistant bacteria) is typically increased compared with a control or reference population, the amount may be increased by at least 10%, at least 20%, at least 30%, at least 40%, at least 50%, at least 60, at least 70%, at least 80%, at least 90%, at least 100%, at least 150%, at least 200% compared with the control or reference population.

Alternatively, if the control or reference population is positive for carbapenemase-producing bacteria and/or a carbapenem-resistant bacterial infection, the amount of the one or more carbapenemase-producing bacteria (i.e. carbapenem-resistant bacteria) may be decreased compared with the control or reference population. For example, the amount may be decreased by at least 10%, at least 20%, at least 30%, at least 40%, at least 50%, at least 60, at least 70%, at least 80%, at least 90%, at least 95%, at least 99%, up to total elimination of the one or more carbapenemase-producing bacteria (carbapenem-resistant bacteria). Such a carbapenem-resistant bacterial infection positive control or reference population may, for example, be used to monitor an individual's response to a treatment directed to the carbapenem-resistant bacterial infection, such that if the treatment is successful, the amount of carbapenemase-producing bacteria will decrease relative to the control or reference population over time.

### Measurement of the One or More Carbapenemase-Producing Bacteria

The presence and/or amount of the one or more carbapenemase-producing bacteria (carbapenem-resistant bacteria) according to the present invention is determined by determining the presence and/or amount of one or more carbapenemase gene produced by the one or more carbapenemase-producing bacteria. If more than one carbapenemase-producing bacteria is present in a sample, the different bacteria may produce the same carbapenemase gene or different carbapenemase genes. Each carbapenemase-producing bacteria according to the present invention may produce one, two, three, four, five, six, seven, eight, nine, ten, 11, 12, 13, 14, 15 or more different carbapenemase genes.

Measurements of the one or more carbapenemase gene may include, for example, measurements that indicate the presence, concentration, expression level, or any other value associated with the one or more carbapenemase gene.

The present and/or amount of said one or more carbapenemase gene may be determined by quantitative and/or qualitative analysis. Thus, the presence of a carbapenemase-producing bacteria or carbapenem-resistant bacterial infection may be determined simply by determining the expression of one or more carbapenemase gene according to the invention (qualitative analysis), with no need to determine the amount of the carbapenemase gene. Alternatively, the amount of the one or more carbapenemase gene may be determined (quantitative analysis).

The amount of the one or more one or more carbapenemase gene encompasses, but is not limited to, the mass of the one or more carbapenemase gene, the molar amount of the one or more carbapenemase gene, the concentration of the one or carbapenemase gene, the molarity of the one or more carbapenemase gene and the copy number of the one or more carbapenemase gene. This amount may be given in any appropriate units. For example, the concentration of the one or more carbapenemase gene may be given in pg/ml, ng/ml or μg/ml.

The presence and/or amount of the one or more carbapenemase gene may be measured directly or indirectly. For example, the copy number of the one or more carbapenemase gene may be determined using recombinase polymerase amplification (RPA), PCR, qPCR or qRT-PCR. The expression level of the one or more carbapenemase gene may be determined, for example using RPA reverse transcription PCR (RT-PCR). In a preferred embodiment RPA is used. RPA may be preferably used to determine the expression level of any combination of carbapenemase genes, additional carbapenemase genes and/or CTX-M β-lactamase genes as described herein. The relative presence and/or amount of the one or more carbapenemase gene relative to a control or reference population may be determined using any appropriate technique. Suitable standard techniques are known in the art.

As used herein, “comparison” includes any means to discern at least one difference in the presence and/or amount of the one or more carbapenemase gene in the individual and the control or reference population. Thus, a comparison may include a visual inspection of chromatographic spectra or numerical data, and a comparison may include arithmetical or statistical comparisons of values assigned to expression of the one or more carbapenemase gene in the individual's sample and the control or reference. Such statistical comparisons include, but are not limited to, applying a decision rule or decision tree. If at least one internal standard is used, the comparison to discern a difference between the individual and the reference or control may also include features of these internal standards, such that the present and/or amount of the one or more carbapenemase gene in the individual's sample is correlated to the internal standards. The comparison can confirm the presence or absence of one or more carbapenemase-producing bacteria (i.e. carbapenem-resistant bacteria), and thus to detect or diagnose a carbapenem-resistant bacterial infection.

The presence and/or amount level of the one or more carbapenemase gene (and hence the one or more carbapenemase-producing bacteria) may be altered compared with a control or reference population for at least 12 hours, at least 24 hours, at least 30 hours, at least 48 hours, at least 72 hours, at least 96 hours, at least 120 hours, at least 144 hours, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks or more.

Although the invention does not require a monitoring period to diagnose a carbapenem-resistant bacterial infection, it will be understood that repeated classifications of the individual, i.e., repeated snapshots, may be taken over time until the individual is no longer at risk. Alternatively, the presence and/or amount of one or more carbapenemase-producing bacteria in a sample obtained from the individual may be compared to presence and/or amount of one or more carbapenemase-producing bacteria in samples obtained from the same individual at different points in time.

As used herein, an “individual” is an animal, preferably a mammal, more preferably a human or non-human primate. The terms “individual,” “subject” and “patient” are used interchangeably herein. The individual can be normal, suspected of having a carbapenem-resistant bacterial infection or at risk of a carbapenem-resistant bacterial infection.

The present invention enables the rapid detection of one or more carbapenemase-producing bacteria and/or a carbapenem-resistant bacterial infection. By way of example, the method of the invention is typically completed within 3 hours, preferably within 1 to 3 hours, 1 to 2 hours or 1 to 1.5 hours.

The presence and/or amount of one or more carbapenemase-producing bacteria, as determined by determining the presence and/or amount of one or more carbapenemase genes may be detected, quantified or determined by any appropriate means.

### Samples

The presence and/or amount of the one or more carbapenemase gene may be determined in a sample obtained from a bacterial isolate or bacterial culture. The presence and/or amount of the one or more carbapenemase gene may be determined in a sample obtained from an environmental sample, for example a swab taken from any environmental site, such as a surface in a hospital. The presence and/or amount of the one or more carbapenemase gene may be determined in a sample obtained from an individual. The sample may be any suitable biological material, for example blood, including whole blood, plasma, saliva, serum, sputum, bronchial alveolar lavage (BAL), tracheal aspirate, urine, cerebral spinal fluid, cells, a cellular extract, a tissue sample, a tissue biopsy, a stool/faeces sample, skin swab, a swab or sample from any body site, including wounds and abscesses, and the like. Typically the sample is from a bacterial isolate/culture or a rectal swab, stool specimen, urine sample and/or blood sample. The precise biological sample that is taken from the individual may vary, but the sampling preferably is minimally invasive and is easily performed by conventional techniques. The biological sample may be taken from the individual before, during, and/or after treatment for a carbapenem-resistant bacterial infection. In one embodiment, the sample is taken after treatment for a carbapenem-resistant bacterial infection has been initiated. Samples of colonies isolated by culture may also be used.

Similar sample collection systems and sampling may be applied for animal samples, either for primary diagnosis or screening for carbapenemase carriage, or in other forms of environmental sample (including sewage, drinking water samples, food etc.).

When samples are taken from a patient or the environment (rather than a bacterial culture or isolate), extraction of the bacterial DNA is typically required prior to testing according to the present invention. Accordingly, the methods of the present invention may comprise a DNA extraction step. In a preferred embodiment a formal and standard DNA extraction method is used to extract the bacterial DNA. Any suitable technique for DNA extraction may be used. Suitable techniques are known in the art, for example spin column and precipitation techniques. Equally, an automated robotic system for DNA extraction may be used.

Concentration of the bacteria from the sample prior to analysis may be required depending on the number of bacteria in the sample. Concentration is achieved using a variety of techniques, including, but not limited to, filtration through suitable cut-off membranes, capture of bacteria on magnetic beads (on the basis of charge, general binding of bacteria cell wall components (e.g. vancomycin to conserved regions of the bacterial cell wall, molecules that bind to bacterial lipid A) or specific ligands specific to particular bacteria (antibodies, aptamers specific for particular surface ligands).

### Methodology

As described herein, the one or more carbapenemase gene of the invention is detected (quantitatively and/or qualitatively) at the nucleic acid level. Thus, the one or more carbapenemase gene may be detected as DNA and/or RNA and may be detected using any appropriate technique. The presence and/or amount of the one or more carbapenemase gene of the invention may be measured directly or indirectly.

Any appropriate agent may be used to determine the presence and/or amount of the one or more carbapenemase gene of the invention. For example, the presence and/or amount of the one or more carbapenemase gene of the invention may be determined using an agent selected from peptides and peptidomimetics, antibodies, small molecules and single-stranded DNA or RNA molecules, as described herein. The relative presence and/or amount of the one or more carbapenemase gene of the invention relative to a control or reference population (see above) may be determined using any appropriate technique. Suitable standard techniques are known in the art.

Typically the determination of the presence and/or amount of the one or more carbapenemase gene is carried out by amplifying said one or more carbapenemase gene, a target region of said one or more carbapenemase gene or a fragment of said gene or target region. Amplification may be carried out using methods and platforms known in the art, for example recombinase polymerase amplification (RPA), PCR (for example, with the use of “Fast DNA Polymerase”, Life Technologies), such as real-time or quantitative PCR (qPCR), block-based PCR, ligase chain reaction, glass capillaries, isothermal amplification methods including loop-mediated isothermal amplification, rolling circle amplification transcription mediated amplification, nucleic acid sequence-based amplification, signal mediated amplification of RNA technology, strand displacement amplification, isothermal multiple displacement amplification, helicase-dependent amplification, single primer isothermal amplification, and circular helicase-dependent amplification. If employed, amplification may be carried using any amplification platform. In some embodiments, PCR, preferably q-PCR is used. In a particularly preferred embodiment RPA is used.

RPA and/or PCR amplification primers are typically employed to amplify approximately 100-400, for example 100-300 or 100-200 base pair regions of the target/complementary nucleic acid that contain the nucleotide targets of the one or more carbapenemase gene of the present invention.

In RPA, in the presence of a suitable recombinase, single-stranded DNA binding proteins, strand-displacing polymerase and DNA precursors (dATP, dCTP, dGTP and dTTP), forward and reverse primers are extended in a 5′ to 3′ direction, thereby initiating the synthesis of new nucleic acid strands that are complementary to the individual strands of the target nucleic acid. The primers thereby drive amplification of target nucleic acid sequences in the one or more carbapenemase gene, thereby generating amplification products comprising said target nucleic acid sequences. The RPA technique is isothermal, meaning that a thermal cycler is not required, making the technique particularly suitable for use as a low-cost point-of-care (POC) test as it can be run on devices with much simpler heating and detection capability.

In PCR, in the presence of a suitable polymerase and DNA precursors (dATP, dCTP, dGTP and dTTP), forward and reverse primers are extended in a 5′ to 3′ direction, thereby initiating the synthesis of new nucleic acid strands that are complementary to the individual strands of the target nucleic acid. The primers thereby drive amplification of target nucleic acid sequences in the one or more carbapenemase gene, thereby generating amplification products comprising said target nucleic acid sequences.

In a preferred embodiment, the oligonucleotide probes disclosed herein are used as primers to amplify the one or more carbapenemase gene, one or more target regions within said one or more carbapenemase gene or a fragment of said carbapenemase gene. All references, description and features herein described in relation to probes of the invention apply equally to primers of the invention. In this embodiment, the probes (acting as primers) are extended from their 3′ ends (i.e. in a 5′-to-′3′) direction. Such an amplification step may be employed in conjunction with a general amplification step (used to amplify all the DNA within a sample to facilitate detection), although typically such a general amplification step is not required for methods using RPA.

The presence and/or amount of the one or more carbapenemase gene may be determined using PCR, preferably qPCR. DNA is isolated from a sample and specific primers are used to amplify the one or more carbapenemase gene (if present in the sample).

In a preferred embodiment, the presence and/or amount of the one or more carbapenemase gene is determined using RPA. DNA is isolated from a sample and specific primers are used to amplify the one or more carbapenemase gene (if present in the sample).

The presence and/or amount of the one or more carbapenemase gene may be determined by determining the presence and/or amount of a target region of the one or more carbapenemase gene and/or the presence and/or amount of one or more fragment of said carbapenemase gene or target region thereof. References herein to determining the presence and/or amount of the one or more carbapenemase gene apply equally to determining the presence and/or amount of a target region of the one or more carbapenemase gene, and/or the presence and/or amount one or more fragment of said carbapenemase gene or target region thereof.

For example, specific primer pairs as disclosed herein for the one or more carbapenemase gene may be used to amplify the one or more carbapenemase gene, a target region thereof or a fragment of the one or more carbapenemase gene or target region thereof. Typically the fragment is from a target region of the one or more carbapenemase gene. The amplification products of (RPA or PCR) reaction may then be visualised by any appropriate means. For example, the amplification products may be separated and visualised by agarose gel electrophoresis. As the molecular weight of the one or more carbapenemase gene or fragment thereof will be known, the presence of the one or more carbapenemase gene or fragment thereof may be readily determined by the band size of the amplification products as run on an agarose gel. This method has the advantage of requiring standard equipment that will be present in most laboratories.

Specific primer pairs as disclosed herein can also be used in with RPA or other techniques such as qPCR to determine the presence and/or amount of the one or more carbapenemase gene, a target region thereof and/or a fragment of the one or more carbapenemase gene or a target region thereof. Non-specific fluorescent dyes may be used in RPA or qPCR according to the present invention. Standard RPA and qPCR methods using such non-specific fluorescent dyes are known in the art. Alternatively, DNA probes specific for the one or more carbapenemase gene of the invention, said probes further comprising a fluorescent reporter, may be used. Said DNA probes may comprise or consist of one or more of the oligonucleotides of the invention as described herein, further comprising a fluorescent reporter. Examples of fluorescent reporters (also referred to as fluorescent tags) are also described herein. Instruments enabling fast on-screen detection of genes by qPCR are commercially available (for example, Taqman®).

Oligonucleotide primers and/or probes for detecting the presence and/or amount of the one or more carbapenemase gene, or any combination thereof as described herein, may be combined into a single multiplex assay. Alternatively, the primers and/or probes may be combined into multiple multiplex assays, such as two or more multiplex assays, three or more multiplex assay, four or more multiplex assays or more. As one non-limiting example, two multiplex reactions may be used, each with 3 primer/probe sets, such as reaction 1: KPC, NDM, OXA-48 and reaction 2: IMP, IMI, VIM. In some preferred embodiments, the detection of the presence and/or amount of the one or more carbapenemase gene, or any combination thereof, is carried out as individual assays on a microfluidic device. The oligonucleotide primers and/or probes used in said assays may be selected from those disclosed herein, and may be used in any combination.

DNA is typically extracted and/or isolated from a sample prior to analysis by a method of the present invention. Preferably the DNA is purified prior to analysis. Any appropriate may be used to extract and/or isolate and/or purify the DNA. Standard techniques are known in the art and commercial kits are available.

Any other appropriate technique for determining the presence and/or amount of the one or more carbapenemase gene, a target region thereof and/or a fragment of one or more carbapenemase gene or a target region thereof may also be used. For example, the presence and/or amount of the one or more carbapenemase gene, a target region thereof and/or a fragment of one or more carbapenemase gene or a target region thereof may be determined using a method selected from nuclear magnetic resonance, nucleic acid arrays, dot blotting, slot blotting, reverse transcription amplification by reverse transcription RPA (RT-RPA), reverse transcription amplification by reverse transcription PCR (RT-PCR) and Northern analysis.

The methods of the present invention also encompass determining the expression of the one or more carbapenemase gene of the invention, a target region thereof and/or a fragment of the one or more carbapenemase gene or a target region thereof, for example using reverse transcription RPA (RT-RPA) or reverse transcription PCR (RT-PCR). Typically mRNA from a sample is extracted and, optionally, purified, prior to analysis by RT-RPA or RT-PCR. Oligonucleotides of the invention may be used as primers for the reverse transcription of the one or more carbapenemase gene, a target region thereof and/or a fragment of one or more carbapenemase gene or a target region thereof, and/or as primers for the amplification of the resulting cDNA. Either standard PCR or qPCR may be used to amplify and analyse the cDNA resulting from the reverse transcription (i.e. either RT-PCR or quantitative RT-PCR, RT-qPCR). Preferably RT-RPA is used.

Other means of detection of the one or more carbapenemase gene at the nucleic acid level include: (i) carbapenemase-specific oligonucleotide DNA or RNA or any other nucleic acid derivative probes bound to a solid surface; (ii) purified RNA (labelled by any method, for example using reverse transcription and amplification) hybridised to probes; (iii) labelling the RNA in a sample by any method and hybridised to probes; (iv) purified RNA hybridised to probes and a second probe (labelled by any method) hybridised to the purified RNA; (v) hybridising probes to RNA in a sample, and a second probe (labelled by any method) which is hybridised to the RNA; (vi) obtaining purified RNA (labelled by any method), and hybridising the purified labelled RNA to probes; (vii) obtaining purified RNA and hybridising the RNA to probes, then using a second probe (labelled by any method) which hybridises to the RNA; (viii) RT-PCR using any primer/probe combination or inter-chelating fluorescent label, for example SyberGreen; (ix) end-point PCR; (x) digital PCR; (xi) sequencing; (xii) array cards (RT-PCR); (xiii) lateral flow devices/methodology; and/or (xiv) digital microfluidics. Other suitable techniques include rapid whole genome sequencing.

DNA or RNA from a sample (either purified or unpurified) may be labelled via any method (typically amplification) and used to interrogate one or more probe immobilised on a surface. The probe may be any length as defined herein. Typically, for RPA-based methods, the oligonucleotide probe is 10 to 60 nucleotides in length, more preferably 20 to 60 nucleotides in length, even more preferably 30 to 60 nucleotides in length, even more preferably 40 to 55 nucleotides in length, and even more preferably 45 to 55 nucleotides in length and most preferably 48 to 50 nucleotides in length. Typically, for PCR-based methods, the oligonucleotide probe is 10 to 60 nucleotides in length, more preferably 10 to 50 nucleotides in length, even more preferably 10 to 40 nucleotides in length, even more preferably 12 to 40 nucleotides in length, and most preferably 15 to 35 nucleotides in length.

Alternatively, one or more probe may be immobilised on a surface and the DNA or RNA from a sample is hybridised to one or more second probe (labelled by any method). The DNA or RNA hybridised with the second (labelled) probe is then used to interrogate the one or more probe immobilised on the surface. Examples of such methodology are known in the art, including the Vantix™ system.

Any appropriate agent for the detection of and/or for the determination of the amount of the one or more carbapenemase gene of the invention, a target region thereof and/or a fragment of one or more carbapenemase gene or a target region thereof may be used. Similarly, any method that allows for the detecting of the one or more carbapenemase gene, a target region thereof and/or a fragment of one or more carbapenemase gene or a target region thereof the quantification, or relative quantification of the one or more carbapenemase gene may be used.

Agents for the detection of or for the determination of the amount of one or more carbapenemase gene may be used to determine the amount of the one or more carbapenemase gene in a sample obtained from the individual. Such agents typically bind to the one or more carbapenemase gene. Such agents may bind specifically to the one or more carbapenemase gene. The agent for the detection of or for the determination of the amount of the one or more carbapenemase gene may be a nucleic acid probe or other binding agent specific for the one or more carbapenemase gene, a target region thereof and/or a fragment of one or more carbapenemase gene or a target region thereof. By specific, it will be understood that the agent binds to the molecule of interest, in this case the one or more carbapenemase gene, with no significant cross-reactivity to any other molecule, particularly any other nucleic acid. For example, an agent or antibody that is specific for one or more OXA-48-like carbapenemase will show no significant cross-reactivity with human neutrophil elastase and/or VIM. Cross-reactivity may be assessed by any suitable method. Cross-reactivity of an agent or for the one or more carbapenemase gene with a molecule other than the one or more carbapenemase gene may be considered significant if the agent or antibody binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the one or more carbapenemase gene. An agent that is specific for the one or more carbapenemase gene may bind to another molecule such as human neutrophil elastase at less than 90%, 85%, 80%, 75%, 70%, 65%, 60%, 55%, 50%, 45%, 40%, 35%, 30%, 25% or 20% the strength that it binds to the one or more carbapenemase gene. Preferably, the agent or antibody binds to the other molecule at less than 20%, less than 15%, less than 10% or less than 5%, less than 2% or less than 1% the strength that it binds to the one or more carbapenemase gene.

The determination of the presence and/or amount of the one or more carbapenemase gene may use of one or more separation methods. For example, suitable separation methods may include a mass spectrometry method, such as electrospray ionization mass spectrometry (ESI-MS), ESI-MS/MS, ESI-MS/(MS)n (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SLMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n. Other mass spectrometry methods may include, inter alia, quadrupole, fourier transform mass spectrometry (FTMS) and ion trap. Other suitable separation methods may include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE) or other chromatography, such as thin-layer, gas or liquid chromatography, or any combination thereof. The sample may be fractionated prior to application of the separation method.

The determination of the presence and/or amount of the one or more carbapenemase gene may not require physical separation of the one or more carbapenemase gene. For example, nuclear magnetic resonance (NMR) spectroscopy may be used to resolve one or more carbapenemase gene from a complex mixture of molecules. An analogous use of NMR to classify tumours is disclosed in Hagberg, NMR Biomed. 11: 148-56 (1998), for example. Additional procedures include nucleic acid amplification technologies, which may be used to generate a profile of the one or more carbapenemase genes without physical separation of individual carbapenemase genes. (See Stordeur et al, J. Immunol. Methods 259: 55-64 (2002) and Tan et al, Proc. Nat'l Acad. Sci. USA 99: 11387-11392 (2002), for example.)

In one embodiment, the total mRNA from a sample of the individual is assayed, and the various mRNA species that are obtained from the sample are used interrogated for the presence of carbapenemase mRNAs, by hybridizing these mRNAs to an array of specific probes for particular carbapenemase genes, which said probes may comprise oligonucleotides or cDNAs, using standard methods known in the art. Alternatively, the mRNAs may be subjected to gel electrophoresis or blotting methods such as dot blots, slot blots or Northern analysis, all of which are known in the art. (See, e.g., Sambrook et al. in “Molecular Cloning, 3rd ed.,” Cold Spring Harbor Laboratory Press, Cold Spring Harbor, N.Y. (2001).) mRNA profiles also may be obtained by reverse transcription followed by amplification and detection of the resulting cDNAs, as disclosed by Stordeur et al, supra, for example. In another embodiment, the profile may be obtained by using a combination of methods, such as a nucleic acid array combined with mass spectroscopy.

### Probes and Primers

Any appropriate detection means can be used to determine the presence and/or amount of the one or more carbapenemase gene of the invention, a target region thereof and/or a fragment of one or more carbapenemase gene or a target region thereof, and hence determine the presence and/or amount of one or more carbapenemase-producing bacteria, as described herein.

Typically the presence of the one or more carbapenemase gene may be detected, and/or the amount of the one or more carbapenemase gene determined using at one or more oligonucleotide specific for the one or more carbapenemase gene. Typically, said oligonucleotide is an oligonucleotide probe or primer.

As well as providing methods for determining the presence and/or amount of one or more carbapenemase-producing bacteria, the invention also provides oligonucleotides, particularly primers, primer pairs and probes suitable for use in such methods. Such oligonucleotides are described in more detail herein. Any reference herein to an oligonucleotide specific for the one or more carbapenemase gene applies equally to an oligonucleotide probe or primer for the same one or more carbapenemase gene. Similarly, any reference herein to an oligonucleotide probe or primer for the one or more carbapenemase gene applies equally to an oligonucleotide specific for the same one or more carbapenemase gene. In addition, any reference or disclosure herein to an oligonucleotide probe of the invention applies equally to an oligonucleotide primer of the invention and any reference or disclosure herein to an oligonucleotide primer of the invention applies equally to an oligonucleotide probe of the invention.

An oligonucleotide specific for the one or more carbapenemase gene of the invention, or a probe or primer of the invention may have at least 80% sequence identity to the one or more carbapenemase gene of the invention, or a target region within said carbapenemase gene, measured over any appropriate length of sequence. Typically the % sequence identity is determined over a length of contiguous nucleic acid residues. An oligonucleotide specific for the one or more carbapenemase gene, or an oligonucleotide probe or primer of the invention may, for example, have at least 80% sequence identity to the one or more carbapenemase gene of the invention, or target region thereof, measured over at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 35, at least 40, at least 50, at least 60, at least 70, at least 80 or more nucleic acid residues, up to the oligonucleotide specific for the one or more carbapenemase gene, or the oligonucleotide probe or primer having at least 80% sequence identity with the one or more carbapenemase gene of the invention, a target region thereof and/or a fragment of one or more carbapenemase gene or a target region thereof, over the entire length of the oligonucleotide specific for the one or more carbapenemase gene, or the oligonucleotide probe or primer. Sequence identity may be determined with respect to any accession number or SEQ ID NO of the one or more carbapenemase gene disclosed herein. For example, sequence identity of an oligonucleotide specific for an OXA-48-like carbapenemase gene, or OXA-48-like oligonucleotide probe or primer may be determined with respect to the sequence of any OXA-48-like accession number or SEQ ID NO disclosed herein. This also applies to the other carbapenemases disclosed herein, for example the sequence identity of a VIM specific oligonucleotide, probe or primer may be determined relative to the sequence of any VIM accession number or SEQ ID disclosed herein, the sequence identity of a KPC specific oligonucleotide, probe or primer may be determined relative to the sequence of any KPC accession number or SEQ ID disclosed herein, the sequence identity of an NDM specific oligonucleotide, probe or primer may be determined relative to the sequence of any NDM accession number or SEQ ID disclosed herein, the sequence identity of an IMI specific oligonucleotide, probe or primer may be determined relative to the sequence of any IMI accession number or SEQ ID disclosed herein, and the sequence identity of an IMP specific oligonucleotide, probe or primer may be determined relative to the sequence of any IMP accession number or SEQ ID disclosed herein, etc. Where an accession number encompasses multiple genes, typically in the context of the present invention reference to such an accession number only refers to the carbapenemase gene within that accession number. Thus, SEQ ID NOs corresponding to the accession numbers herein typically comprise or consist of the relevant carbapenemase gene sequence.

An oligonucleotide specific for the one or more carbapenemase gene, or an oligonucleotide probe or primer of the invention may be complementary to the one or more carbapenemase gene of the invention, or a target region thereof. Typically the oligonucleotide specific for the one or more carbapenemase gene, or the oligonucleotide probe or primer of the invention is complementary over a length of contiguous nucleic acid residues. An oligonucleotide specific for the one or more carbapenemase gene, or an oligonucleotide probe or primer of the invention may, for example, be complementary to the one or more carbapenemase gene of the invention, or target region thereof, measured over at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 70, at least 80, at least 90, or more nucleic acid residues, up to the oligonucleotide specific for the one or more carbapenemase gene, or the oligonucleotide probe or primer being complementary to the one or more carbapenemase gene of the invention, or target region thereof, over the entire length of the oligonucleotide specific for the one or more carbapenemase gene, or the oligonucleotide probe or primer.

An oligonucleotide specific for the one or more carbapenemase gene, or an oligonucleotide probe or primer of the invention may be complementary to the reverse sequence of the one or more carbapenemase gene of the invention, or a target region thereof. Typically the oligonucleotide specific for the one or more carbapenemase gene, or the oligonucleotide probe or primer of the invention is complementary over a length of contiguous nucleic acid residues of the reverse sequence. An oligonucleotide specific for the one or more carbapenemase gene, or an oligonucleotide probe or primer of the invention may, for example, be complementary to the reverse sequence of the one or more carbapenemase gene of the invention, or target region thereof, measured over at least 10, at least 11, at least 12, at least 13, at least 14, at least 15, at least 16, at least 14, at least 18, at least 19, at least 20, at least 21, at least 22, at least 23, at least 24, at least 25, at least 26, at least 27, at least 28, at least 29, at least 30, at least 35, at least 36, at least 37, at least 38, at least 39, at least 40, at least 45, at least 50, at least 55, at least 60, at least 70, at least 80, at least 90, or more nucleic acid residues, up to the oligonucleotide specific for the one or more carbapenemase gene, or the oligonucleotide probe or primer being complementary to the reverse sequence of the one or more carbapenemase gene of the invention, or target region thereof, over the entire length of the oligonucleotide specific for the one or more carbapenemase gene, or the oligonucleotide probe or primer.

An oligonucleotide specific for the one or more carbapenemase gene, or an oligonucleotide probe or primer of the invention may be complementary to a variant of the one or more carbapenemase gene of the invention, or a variant of a target region of said carbapenemase gene. Typically the oligonucleotide specific for the one or more carbapenemase gene, or the oligonucleotide probe or primer is complementary to a variant having at least 80% sequence identity to the one or more carbapenemase gene of the invention, or a variant having at least 80% sequence identity to the target region of said carbapenemase gene. The % sequence identity of the variant to the one or more carbapenemase gene of the invention, or a variant of a target region of said carbapenemase gene may be calculated over any appropriate length of sequence in the one or more carbapenemase gene, as described herein.

A sequence identity of at least 80% includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO presented herein).

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. MoI. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Wale et al., Align-M—A New Algorithm for Multiple Alignment of Highly Divergent Sequences, 20 (9) Bioinformatics:1428-1435 (2004). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992.

Variants of the specific sequences provided above may alternatively be defined by reciting the number of nucleotides that differ between the variant sequences and the specific reference sequences provided. These differences may result from the addition, deletion and/or substitution of one or more nucleotide position within the variant sequence compared with the reference sequence. Thus, in one embodiment, the sequence may comprise (or consist of) a nucleotide sequence that differs from the specific sequences provided at no more than ten nucleotide positions, no more than nine nucleotide positions, no more than eight nucleotide positions, no more than seven nucleotide positions, no more than six nucleotide positions, no more than five nucleotide positions, no more than four nucleotide positions, no more than three nucleotide positions, no more than two nucleotide positions or no more than one nucleotide position. Conservative substitutions are preferred. The term variants as defined herein also encompasses splice variants.

An oligonucleotide specific for the one or more carbapenemase gene, or an oligonucleotide primer of the invention may be at least ten, at least 11, at least 12, at least 13, at least 14, at least 15, at least 16, at least 17, at least 18, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65 or more nucleotides in length. Typically, for RPA-based methods, the oligonucleotide probe is 10 to 60 nucleotides in length, more preferably 20 to 60 nucleotides in length, even more preferably 30 to 60 nucleotides in length, even more preferably 40 to 55 nucleotides in length, and even more preferably 45 to 55 nucleotides in length and most preferably 48 to 50 nucleotides in length. Typically, for PCR-based methods, the oligonucleotide probe is 10 to 60 nucleotides in length, more preferably 10 to 50 nucleotides in length, even more preferably 10 to 40 nucleotides in length, even more preferably 12 to 40 nucleotides in length, and most preferably 15 to 35 nucleotides in length.

An oligonucleotide probe of the invention may be at least ten, at least 11, at least 12, at least 13, at least 14, at least 15, at least 16, at least 17, at least 18, at least 20, at least 21, at least 22, at least 23, at least 24, at least 25, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 80, at least 90, at least 100, or more nucleotides in length. In a preferred embodiment, the oligonucleotide probe is 10 to 70 nucleotides in length, more preferably 20 to 60 nucleotides in length, even more preferably 30 to 60 nucleotides in length, even more preferably 40 to 60 nucleotides in length, even more preferably 40 to 50 nucleotides in length, and most preferably 45 to 50 nucleotides in length.

The oligonucleotides specific for the one or more carbapenemase gene, or the oligonucleotide probes and primers of the invention are typically designed to hybridise to their target nucleic acid sequence present in the one or more carbapenemase gene of the invention. In the context of the present invention, the term hybridises includes hybridising to the sense strand of a target sequence, the reverse of a target sequence, the complement of a target sequence or the reverse complement of a target sequence. Further, references herein to an oligonucleotide specific for the one or more carbapenemase gene, or the oligonucleotide probes and primers of the invention comprising a particular sequence also encompass oligonucleotides, probes and primers consisting of said sequences, as well as oligonucleotides comprising or consisting of the complement or reverse complement of said sequences. In a preferred embodiment, reverse primers and/or probes of the invention are reverse complement sequences and so are shown in this orientation. Also in the context of the present invention, the terms “hybridise” and “bind” may be used interchangeably.

Any reference herein to a target sequence (for example an OXA-48-like or any other carbapenemase gene) defined in terms of the bases of a particular sequence applies equally to sub-ranges within the recited bases. Thus, definitions of an oligonucleotide specific to said target region may apply equally to oligonucleotides specific for a sub-range of bases within the recited target region. As a non-limiting example, a reference herein to a target region of an OXA gene comprising bases 120 to 180 of said gene applies equally to a target region of said OXA gene comprising bases 120 to 180 or 130 to 180 or 120 to 170 or 130 to 170, or any other sub-range within the 120 to 180 target region. As a further non-limiting example, an oligonucleotide specific for a target region of an OXA gene comprising bases 120 to 180 of said gene applies equally to a an oligonucleotide specific for a target region of said OXA gene comprising bases 120 to 180 or 130 to 180 or 120 to 170 or 130 to 170, or any other sub-range within the 120 to 180 target region.

An OXA-48-like gene having at least 80%, at least 85%, at least 90%, at least 95%, at least 96%, at least 97%, at least 98%, at least 99% or more sequence identity with one or more of the following OXA carbapenemases (at the amino acid or nucleic acid level): OXA-181 (SEQ ID NO: 43, Accession No. GI 304368222, HM992946.1), OXA-232 (SEQ ID NO: 44, GI 444236140, JX423831.1, 2677-3474,), OXA-247 (SEQ ID NO: 45, GI 442769982, JX893517.1, 1-786), OXA-163 (SEQ ID NO: 46, GI 323817046, HQ700343.1, 1-786), OXA-204 (SEQ ID NO: 49, GI 408795221, JQ809466.1, 5375-6172), OXA-370 (SEQ ID NO: 51, GI 573006828, KF900153.1, 1-798), OXA-245 (SEQ ID NO: 52, GI 442577759, JX438001.1, 1-798), OXA-162 (SEQ ID NO: 53, GI 270312218, GU197550.1, 1-798), OXA-48 (SEQ ID NO: 55, GI AY236073.2, 2188-2985) and/or OXA-244 (SEQ ID NO: 64, GI 442577757, JX438000.1, 1-798), or the OXA-48-like gene of one of these accession numbers may be used as a reference sequence to determine the positions of the target regions of the OXA-48-like gene. For example, the target sequences may be those from one or more of these accession numbers, or regions corresponding to these target regions from variants of these accession numbers or from another OXA gene (e.g. from another OXA accession number disclosed herein).

Alternatively, the OXA gene of accession number JN714122 (SEQ ID NO: 58), JQ99150 (SEQ ID NO: 41) and/or JQ809466 (SEQ ID NO: 49) may be used as a reference OXA gene sequence to determine the positions of the target regions of the OXA-48 gene. For example, the target sequences may be those from one or more of accession numbers JN714122 (SEQ ID NO: 58), JQ99150 (SEQ ID NO: 41) and/or JQ809466 (SEQ ID NO: 49), or regions corresponding to these target regions from variants of these accession numbers or from another OXA gene (e.g. from another OXA accession number disclosed herein).

According to the present invention, one or more additional oligonucleotide specific for an OXA-48-like gene may be used. Said one or more additional oligonucleotide specific for the OXA-48-like gene may hybridise to a nucleic acid sequence within a target region of the OXA-48-like gene comprising bases 100 to 400, preferably 100 to 360, even more preferably 120 to 360 and most preferably 120 to 320 of the OXA-48-like gene as defined by any of the OXA-48-like accession numbers or SEQ ID NOs disclosed herein or the complement or reverse complement thereof (or the sequence of any other OXA-48-like gene, the accession numbers and SEQ ID NOs disclosed herein are not intended as an exhaustive list of OXA-48-like sequences).

Typically: (i) a first oligonucleotide sequence specific for the OXA-48-like gene hybridises to a nucleic acid sequence within a target region of the OXA-48-like gene comprising bases 100 to 180, preferably 120 to 180 and more preferably 130 to 170 of the OXA-48-like gene; (ii) a second oligonucleotide sequence specific for the OXA-48-like gene hybridises to a nucleic acid sequence within a target region of the OXA-48-like gene comprising bases 250 to 360, preferably 250 to 320, more preferably 260 to 320 of the OXA-48-like gene; and/or (iii) a third oligonucleotide sequence specific for the OXA-48-like gene hybridises to a nucleic acid sequence within a target region of the OXA-48-like gene comprising bases 180 to 250, preferably 180 to 240 and more preferably 190 to 240 of the OXA-48-like gene. In some embodiments, said first oligonucleotide is a forward primer, said second oligonucleotide is a reverse primer, and/or said third oligonucleotide is a probe The reference to bases/positions with a OXA-48-like gene may be defined in terms of any of the OXA-48-like accession numbers or SEQ ID NOs disclosed herein or the complement or reverse complement thereof, or the sequence of any other OXA-48-like gene. As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the OXA-48-like specific oligonucleotides (i) and/or (ii) are used, preferably (i) and (ii). In a more preferred embodiment, all three OXA-48-like specific oligonucleotides (i), (ii) and (iii) are used according to the present invention. Typically all three OXA-48-like specific oligonucleotides (ii) and (iii) are used when recombinase polymerase amplification (RPA) is used to determine the presence and/or amount of one or more OXA-48-like gene.

A VIM gene having at least 80%, at least 85%, at least 90%, at least 95%, at least 96%, at least 97%, at least 98%, at least 99% or more sequence identity with one or more of the following VIM carbapenemases (at the amino acid or nucleic acid level) with the VIM gene of accession number Y18050 (SEQ ID NO: 110), AF191564 (SEQ ID NO: 87) and/or AJ536835 (SEQ ID NO: 65) may be used as a reference VIM gene sequence to determine the positions of the target regions of the VIM gene. For example, the target sequences may be those from one or more of these accession numbers, or regions corresponding to these target regions from variants of these accession numbers or from another VIM gene (e.g. from another VIM accession number disclosed herein).

According to the present invention, one or more oligonucleotide specific for a VIM gene may be used. Said one or more oligonucleotide specific for the VIM gene may hybridise or bind to a nucleic acid sequence within a target region of a VIM gene comprising bases 120 to 195, preferably 120 to 180, more preferably 130 to 180, even more preferably 140 to 175 of any of the VIM accession numbers or SEQ ID NOs disclosed herein or the complement or reverse complement thereof (or the sequence of any other VIM gene, the accession numbers and SEQ ID NOs disclosed herein are not intended as an exhaustive list of VIM sequences) (i). A VIM specific oligonucleotide, probe or primer of the invention may comprise a region of at least 34, at least 35 or more contiguous bases from SEQ ID NO: 25, or from a nucleic acid sequence having at least 95% sequence identity to the full-length of SEQ ID NO: 25, and hybridise to a nucleic acid sequence within a target region of the VIM gene comprising bases 270 to 330, preferably 280 to 320 of the VIM gene (ii). A VIM specific oligonucleotide, probe or primer of the invention may be from about 30 to 60 bases in length and hybridise to a nucleic acid sequence within a target region of the VIM gene comprises bases 190 to 270, preferably 210 to 270, even more preferably 210 to 265 of the VIM gene (iii). For example, said oligonucleotide (iii) may hybridise to a nucleic acid sequence within a target region of the VIM gene comprising bases 210 to 235 of the VIM gene.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the VIM specific oligonucleotides (i) and/or (ii) are used, preferably (i) and (ii). In a more preferred embodiment, all three VIM specific oligonucleotides (i), (ii) and (iii) are used according to the present invention. Typically all three VIM specific oligonucleotides (i), (ii) and (iii) are used when recombinase polymerase amplification (RPA) is used to determine the presence and/or amount of one or more VIM gene.

Typically: (i) a first oligonucleotide sequence specific for the VIM gene hybridises to a nucleic acid sequence within a target region of the VIM gene comprising bases 130 to 180, preferably 140 to 180 of the VIM gene; (ii) a second oligonucleotide sequence specific for the VIM gene comprises a region of at least 34 contiguous bases from SEQ ID NO: 25, or from a nucleic acid sequence having at least 95% sequence identity to the full-length of SEQ ID NO: 25, and which hybridises to a nucleic acid sequence within a target region of the VIM gene comprising bases 283 to 318 of the VIM gene; and/or (iii) a third oligonucleotide sequence specific for the VIM gene is 30 to 60 bases in length and hybridises to a nucleic acid sequence within a target region of the VIM gene comprises bases 210 to 270, preferably 210 to 265 of the VIM gene. In some embodiments, said first oligonucleotide is a forward primer, said second oligonucleotide is a reverse primer, and/or said third oligonucleotide is a probe The reference to bases/positions with a VIM gene may be defined in terms of any of the VIM accession numbers or SEQ ID NOs disclosed herein or the complement or reverse complement thereof, or the sequence of any other VIM gene. As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the VIM specific oligonucleotides (i) and/or (ii) are used, preferably (i) and (ii). In a more preferred embodiment, all three VIM specific oligonucleotides (i), (ii) and (iii) are used according to the present invention. Typically all three VIM specific oligonucleotides (i), (ii) and (iii) are used when recombinase polymerase amplification (RPA) is used to determine the presence and/or amount of one or more VIM gene.

A KPC gene having at least 80%, at least 85%, at least 90%, at least 95%, at least 96%, at least 97%, at least 98%, at least 99% or more sequence identity with one or more of the following KPC carbapenemases (at the amino acid or nucleic acid level) with the KPC gene of accession number AF297554 (SEQ ID NO: 120) may be used as a reference KPC gene sequence to determine the positions of the target regions of the KPC gene. For example, the target sequences may be those from one or more of these accession numbers, or regions corresponding to these target regions from variants of these accession numbers or from another KPC gene (e.g. from another KPC accession number disclosed herein).

According to the present invention, one or more oligonucleotide specific for a KPC gene may be used. Said one or more oligonucleotide specific for the KPC gene may hybridise to a nucleic acid sequence within a target region of the KPC gene comprising bases 100 to 310, preferably 120 to 310 of the KPC gene as defined by any of the KPC accession numbers or SEQ ID NOs disclosed herein or the complement or reverse complement thereof (or the sequence of any other KPC gene, the accession numbers and SEQ ID NOs disclosed herein are not intended as an exhaustive list of KPC sequences).

Typically: (i) a first oligonucleotide sequence specific for the KPC gene hybridises to a nucleic acid sequence within a target region of the KPC gene comprising bases 100 to 180, preferably 120 to 180, more preferably 120 to 160 of the KPC gene; (ii) a second oligonucleotide sequence specific for the KPC gene hybridises to a nucleic acid sequence within a target region of the KPC gene comprising bases 240 to 310, preferably 250 to 310, more preferably 260 to 310, even more preferably 270 to 310 of the KPC gene; and/or (iii) a third oligonucleotide sequence specific for the KPC gene hybridises to a nucleic acid sequence within a target region of the KPC gene comprising bases 160 to 270, preferably 160 to 240, more preferable 180 to 240 of the KPC gene. In some embodiments, said first oligonucleotide is a forward primer, said second oligonucleotide is a reverse primer, and/or said third oligonucleotide is a probe The reference to bases/positions with a KPC gene may be defined in terms of any of the KPC accession numbers or SEQ ID NOs disclosed herein or the complement or reverse complement thereof, or the sequence of any other KPC gene. As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the KPC specific oligonucleotides (i) and/or (ii) are used, preferably (i) and (ii). In a more preferred embodiment, all three KPC specific oligonucleotides (i), (ii) and (iii) are used according to the present invention. Typically all three KPC specific oligonucleotides (i), (ii) and (iii) are used when recombinase polymerase amplification (RPA) is used to determine the presence and/or amount of one or more KPC gene.

An NDM gene having at least 80%, at least 85%, at least 90%, at least 95%, at least 96%, at least 97%, at least 98%, at least 99% or more sequence identity with one or more of the following NDM carbapenemases (at the amino acid or nucleic acid level) with the NDM gene of accession number FN396876 (SEQ ID NO: 143) may be used as a reference NDM gene sequence to determine the positions of the target regions of the NDM gene. For example, the target sequences may be those from one or more of these accession numbers, or regions corresponding to these target regions from variants of these accession numbers or from another NDM gene (e.g. from another NDM accession number disclosed herein).

According to the present invention, one or more oligonucleotide specific for an NDM gene may be used. Said one or more oligonucleotide specific for the NDM gene may hybridise to a nucleic acid sequence within a target region of the NDM gene comprising bases 350 to 480, or may be at least 25 bases in length and hybridise to a nucleic acid sequence within a target region of the NDM gene comprising bases 250 to 480, as defined by any of the KPC accession numbers or SEQ ID NOs disclosed herein or the complement or reverse complement thereof (or the sequence of any other KPC gene, the accession numbers and SEQ ID NOs disclosed herein are not intended as an exhaustive list of KPC sequences).

Said one or more oligonucleotide specific for the NDM gene may comprise at least 25 bases in length and hybridises to a nucleic acid sequence within a target region of the NDM gene comprising bases 250 to 350, preferably 280 to 330 of the NDM gene (i). An NDM specific oligonucleotide, probe or primer of the invention may hybridise to a nucleic acid sequence within a target region of the NDM gene comprising bases 400 to 480, preferably 400 to 450, more preferably 410 to 450 of the NDM gene (ii). An NDM specific oligonucleotide, probe or primer of the invention may hybridise to a nucleic acid sequence within a target region of the NDM gene comprising bases 330 to 410, preferably 350 to 410, more preferably 360 to 410 of the NDM gene (iii). The NDM gene may be defined by any of the NDM accession numbers or SEQ ID NOs disclosed herein or the complement or reverse complement thereof (or the sequence of any other NDM gene, the accession numbers and SEQ ID NOs disclosed herein are not intended as an exhaustive list of NDM sequences). As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the NDM specific oligonucleotides (i) and/or (ii) are used, preferably (i) and (ii). In a more preferred embodiment all three NDM specific oligonucleotides (i), (ii) and (iii) are used according to the present invention. Typically all three NDM specific oligonucleotides (i), (ii) and (iii) are used when recombinase polymerase amplification (RPA) is used to determine the presence and/or amount of one or more NDM gene.

Typically: (i) a first oligonucleotide sequence specific for the NDM gene is at least 25, at least 30, at least 31, at least 32, at least 33, at least 34, or at least 35 bases in length and hybridises to a nucleic acid sequence within a target region of the NDM gene comprising bases 250 to 350, preferably 280 to 330 of the NDM gene; (ii) a second oligonucleotide sequence specific for the NDM gene hybridises to a nucleic acid sequence within a target region of the NDM gene comprising bases 410 to 450 of the NDM gene; and/or (iii) a third oligonucleotide sequence specific for the NDM gene hybridises to a nucleic acid sequence within a target region of the NDM gene comprising bases 360 to 410 of the NDM gene. In some embodiments, said first oligonucleotide is a forward primer, said second oligonucleotide is a reverse primer, and/or said third oligonucleotide is a probe. The reference to bases/positions with an NDM gene may be defined in terms of any of the NDM accession numbers or SEQ ID NOs disclosed herein or the complement or reverse complement thereof, or the sequence of any other NDM gene. As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the NDM specific oligonucleotides (i) and/or (ii) are used, preferably (i) and (ii). In a more preferred embodiment, all three NDM specific oligonucleotides (i), (ii) and (iii) are used according to the present invention. Typically all three NDM specific oligonucleotides (i), (ii) and (iii) are used when recombinase polymerase amplification (RPA) is used to determine the presence and/or amount of one or more NDM gene.

An IMP gene having at least 80%, at least 85%, at least 90%, at least 95%, at least 96%, at least 97%, at least 98%, at least 99% or more sequence identity with one or more of the following IMP carbapenemases (at the amino acid or nucleic acid level) with the IMP gene of accession number S71932 (SEQ ID NO: 204) may be used as a reference IMP gene sequence to determine the positions of the target regions of the IMP gene. For example, the target sequences may be those from one or more of these accession numbers, or regions corresponding to these target regions from variants of these accession numbers or from another IMP gene (e.g. from another IMP accession number disclosed herein).

According to the present invention, one or more oligonucleotide specific for an IMP gene may be used. Said one or more oligonucleotide specific for the IMP gene may hybridise to a nucleic acid sequence within a target region of the IMP gene comprising bases 730 to 783, preferably 750 to 783 of the IMP gene (i).

A (second) IMP specific oligonucleotide, probe or primer of the invention may hybridise to a nucleic acid sequence within a target region of the IMP gene comprising bases 850 to 886, preferably 870 to 886 of the IMP gene. Alternatively, an IMP specific oligonucleotide, probe or primer of the invention may comprise or consist of any one of SEQ ID NOs: 12-22, or a nucleic acid sequence having at least 80% sequence identity to the full-length of any one of SEQ ID NOs: 12-22, and hybridise to a nucleic acid sequence within a target region of the IMP gene comprising base 850 to 920, preferably 870 to 910 of the IMP gene (ii).

A (third) IMP specific oligonucleotide, probe or primer of the invention may hybridise to a nucleic acid sequence within a target region of the IMP gene comprising bases 802 to 860, preferably 802 to 840 of the IMP gene. Preferably, an IMP specific oligonucleotide, probe or primer of the invention may comprise at least 30 bases and hybridise to a nucleic acid sequence within a target region of the IMP gene comprising base 784 to 860, preferably 784 to 840 of the IMP gene. Optionally, said IMP specific oligonucleotide, probe or primer comprises a region of at least 30, at least 35, at least 40, at least 45 or more contiguous bases from SEQ ID NO: 23, or from a nucleic acid sequence having at least 80% sequence identity to the full-length of SEQ ID NO: 23 (iii).

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the IMP specific oligonucleotides (i) and/or (ii) are used, preferably (i) and (ii). In a more preferred embodiment, all three IMP specific oligonucleotides (i), (ii) and (iii) are used according to the present invention. Typically all three IMP specific oligonucleotides (i), (ii) and (iii) are used when recombinase polymerase amplification (RPA) is used to determine the presence and/or amount of one or more IMP gene.

Typically: (i) a first oligonucleotide sequence specific for the IMP gene hybridises to a nucleic acid sequence within a target region of the IMP gene comprising bases 750 to 783 of the IMP gene; (ii) a second oligonucleotide sequence specific for the IMP gene comprises any one of SEQ ID NOs: 12-22, or a nucleic acid sequence having at least 80% sequence identity to the full-length of any one of SEQ ID NOs: 12-22, and hybridises to a nucleic acid sequence within a target region of the IMP gene comprising bases 850 to 886, preferably 870 to 998 of the IMP gene; and/or (iii) a third oligonucleotide sequence specific for the IMP gene is at least 30 bases in length and hybridises to a nucleic acid sequence within a target region of the IMP gene comprising bases 784 to 860, preferably 784 to 840 of the IMP gene.

In some embodiments, said first oligonucleotide is a forward primer, said second oligonucleotide is a reverse primer, and/or said third oligonucleotide is a probe. The reference to bases/positions with an IMP gene may be defined in terms of any of the IMP accession numbers or SEQ ID NOs disclosed herein or the complement or reverse complement thereof, or the sequence of any other IMP gene. As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the IMP specific oligonucleotides (i) and/or (ii) are used, preferably (i) and (ii). In a more preferred embodiment, all three IMP specific oligonucleotides (i), (ii) and (iii) are used according to the present invention. Typically all three IMP specific oligonucleotides (i), (ii) and (iii) are used when recombinase polymerase amplification (RPA) is used to determine the presence and/or amount of one or more IMP gene.

An IMI gene having at least 80%, at least 85%, at least 90%, at least 95%, at least 96%, at least 97%, at least 98%, at least 99% or more sequence identity with one or more of the following IMI carbapenemases (at the amino acid or nucleic acid level) with the IMI gene of accession number DQ173429 (SEQ ID NO: 207) may be used as a reference IMI gene sequence to determine the positions of the target regions of the IMI gene. For example, the target sequences may be those from one or more of these accession numbers, or regions corresponding to these target regions from variants of these accession numbers or from another IMI gene (e.g. from another IMI accession number disclosed herein).

According to the present invention, one or more oligonucleotide specific for an IMI gene may be used. Said one or more oligonucleotide specific for the IMI gene may hybridise to a nucleic acid sequence within a target region of the IMI gene comprising bases 410 to 446, preferably 418 to 446 of the IMI gene (i).

A (second) IMI specific oligonucleotide, probe or primer of the invention may hybridise to a nucleic acid sequence within a target region of the IMI gene comprising bases 507 to 550, preferably 507 to 537 of the IMI gene. Alternatively, an IMI specific oligonucleotide, probe or primer of the invention may comprise or consist of SEQ ID NO: 29 or 30, or a nucleic acid sequence having at least 80% sequence identity to the full-length of SEQ ID NO: 29 or 30, and hybridise to a nucleic acid sequence within a target region of the IMP gene comprising base 517 to 560, preferably 517 to 545 of the IMI gene (ii).

A (third) IMI specific oligonucleotide, probe or primer of the invention may hybridise to a nucleic acid sequence within a target region of the IMI gene comprising bases 452 to 505, preferably 452 to 500 of the IMI gene. Optionally, said IMI specific oligonucleotide, probe or primer comprises a region of at least 30, at least 35, at least 40, at least 45 or more contiguous bases from SEQ ID NO: 207, or from a nucleic acid sequence having at least 80% sequence identity to the full-length of SEQ ID NO: 207 (iii).

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, all three IMI specific oligonucleotides (i), (ii) and (iii) are used according to the present invention.

In one embodiment, the oligonucleotide specific for the one or more carbapenemase gene, or an oligonucleotide primer or probe of the invention is designed against a consensus target sequence from different variants of the one or more carbapenemase gene. Thus, the oligonucleotide, primer and/or probe may comprise or consist of a sequence corresponding to a consensus target sequence, or a sequence having a defined sequence identity with said consensus target sequence. Alternatively, the oligonucleotide, primer and/or probe may be complementary or reverse complementary to a consensus target sequence, or a sequence having a defined sequence identity with said consensus target sequence. Thus, the oligonucleotide, primer and/or probe may hybridise or bind to the consensus target sequence or the complement or reverse complement thereof, or the oligonucleotide, primer and/or probe may hybridise or bind to a sequence having a defined sequence identity with said consensus target sequence, or complement or reverse complement thereof.

As a non-limiting example, an oligonucleotide specific for an OXA-48-like carbapenemase gene, or an oligonucleotide primer or probe for an OXA-48-like carbapenemase gene may be designed against a consensus target sequence, wherein the consensus is generated across the corresponding target sequence of each member of the OXA-48-like family. Thus, an oligonucleotide specific for an OXA-48-like carbapenemase gene, or an oligonucleotide primer or probe for an OXA-48-like carbapenemase gene may bind to or hybridise not only the consensus target sequence or the complement or reverse complement thereof, but also to sequences having a defined % sequence identity with said consensus target sequence or complement or reverse complement thereof. Typically an oligonucleotide specific for an OXA-48-like carbapenemase gene, or an oligonucleotide primer or probe for an OXA-48-like carbapenemase gene will bind to or hybridise target sequences corresponding to the consensus sequence (or the complement or reverse complement thereof) in all members of the OXA-48-like carbapenemase family. This applies equally to the other carbapenemase families of the invention, including VIM, KPC, NDM, IMP, IMI and/or other carbapenemase genes.

Typically the oligonucleotide specific for an OXA-48-like carbapenemase gene, or the oligonucleotide primer or probe for an OXA-48-like carbapenemase gene has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with any one target sequence from the OXA-48-like gene family, or to the complement or reverse complement of said target sequence. In a preferred embodiment the oligonucleotide primer or probe for an OXA-48-like carbapenemase gene has at least 85%, at least 88%, at least 89%, at least 90%, at least 91%, at least 92%, at least 93%, at least 94%, at least 95%, at least 96%, at least 97%, at least 98%, at least 99% or more sequence identity with any one target sequence from the OXA-48-like gene family, or to the complement or reverse complement of said target sequence.

Wherein the present invention relates to determining the presence and/or amount of an OXA-48-like gene, the at least one oligonucleotide specific for the OXA-48-like gene may comprise or consist of:

(i) a region of at least 24, at least 25, at least 26, at least 27, at least 28, at least 29 or the full-length of 30 contiguous bases from SEQ ID NO: 4, or from a nucleic acid sequence having at least 80% sequence identity to the full-length of the nucleic acid sequence of SEQ ID NO: 4 (as defined herein) or a nucleic acid which is complementary to SEQ ID NO: 4 or a nucleic acid having at least 80% sequence identity to the full-length of the nucleic acid sequence of SEQ ID NO: 4. Said oligonucleotides hybridise to a target region of the OXA-48-like gene comprising bases 100 to 180, preferably 120 to 180 and more preferably 130 to 170 of the OXA-48-like gene. Typically, this oligonucleotide specific for the OXA-48-like gene is a forward primer.

(ii) a region of at least 24, at least 25, at least 26, at least 27, at least 28, at least 29, at least 30 or the full-length of 31 contiguous bases from SEQ ID NO: 5, or from a nucleic acid sequence having at least 80% sequence identity to the full-length of the nucleic acid sequence of SEQ ID NO: 5 (as defined herein) or a nucleic acid which is complementary to SEQ ID NO: 5 or a nucleic acid having at least 80% sequence identity to the full-length of the nucleic acid sequence of SEQ ID NO: 5. Said oligonucleotides hybridise to a target region of the OXA-48-like gene comprising bases 250 to 360, preferably 250 to 320, more preferably 260 to 320 of the OXA-48-like gene. Typically, this oligonucleotide specific for the OXA-48-like gene is a reverse primer.

(iii) comprise a region of at least 25, at least 30, at least 35, at least 40, at least 41, at least 42, at least 43, at least 44, at least 45, at least 46, at least 47, or the full-length of 48 contiguous bases from SEQ ID NO: 6, or from a nucleic acid sequence having at least 80% sequence identity to the full-length of the nucleic acid sequence of SEQ ID NO: 6 (as defined herein) or a nucleic acid which is complementary to SEQ ID NO: 6 or a nucleic acid having at least 80% sequence identity to the full-length of the nucleic acid sequence of SEQ ID NO: 6. Said oligonucleotides hybridise to a target region of the OXA-48-like gene comprising bases 180 to 250, preferably 180 to 240 and more preferably 190 to 240 of the OXA-48-like gene. Typically, this oligonucleotide specific for the OXA-48-like gene is a probe.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the forward and/or reverse primers are used, more preferably the forward and reverse primers. In an even more preferred embodiment, all three OXA-48-like specific oligonucleotides (i), (ii) and (iii) may be used according to the present invention.

The oligonucleotide(s) specific for the OXA-48-like gene may be used in combination with any of the other oligonucleotides disclosed herein, including other oligonucleotides specific for the OXA-48-like gene, and/or oligonucleotides specific for one or more other carbapenemase genes, including but not limited to a VIM gene, a KPC gene, an NDM gene, an IMP gene, an IMI gene, a GES gene and/or an SPM gene.

One or more additional oligonucleotide specific for the OXA-48-like gene may be used to determine the presence and/or amount of the OXA-48-like gene in addition to the specific oligonucleotide defined above. Said one or more additional oligonucleotide specific for the OXA-48-like gene may comprise any other oligonucleotide specific for an OXA-48-like gene as described herein. Said one or more additional oligonucleotide specific for the OXA-48-like gene may be used in any combination with any of the oligonucleotides specific for any other carbapenemase gene (examples of other carbapenemase genes and oligonucleotides specific for said other carbapenemase genes are described herein), particularly the oligonucleotides specific for the VIM and/or NDM and/or KPC and/or IMP and/or IMI carbapenemase genes as described herein.

Wherein the present invention relates to determining the presence and/or amount of a VIM gene, the at least one oligonucleotide specific for the VIM gene may comprise or consist of:

(i) a region of at least 24, at least 25, at least 26, at least 27, at least 28, at least 29, at least 30, at least 31, at least 32 or the full-length of 33 contiguous bases from SEQ ID NO: 24, or from a nucleic acid sequence having at least 80% sequence identity to the full-length of the nucleic acid sequence of SEQ ID NO: 24 (as defined herein) or a nucleic acid which is complementary to SEQ ID NO: 24 or a nucleic acid having at least 80% sequence identity to the full-length of the nucleic acid sequence of SEQ ID NO: 24. Said oligonucleotides hybridise to a target region of the VIM gene comprising bases 120 to 195, preferably 120 to 180, more preferably 130 to 180, even more preferably 140 to 175 of the VIM. Typically, this oligonucleotide specific for the VIM gene is a forward primer.

(ii) a region of at least 24, at least 25, at least 26, at least 27, at least 28, at least 29, at least 30, at least 31, at least 32, at least 33, at least 34 or the full-length of 35 contiguous bases from SEQ ID NO: 25, or from a nucleic acid sequence having at least 95% sequence identity to the full-length of the nucleic acid sequence of SEQ ID NO: 25 (as defined herein) or a nucleic acid which is complementary to SEQ ID NO: 25 or a nucleic acid having at least 95% sequence identity to the full-length of the nucleic acid sequence of SEQ ID NO: 25. Said oligonucleotides hybridise to a target region of the VIM gene comprising bases 270 to 330, preferably 280 to 320 of the VIM gene. Typically, this oligonucleotide specific for the VIM gene is a reverse primer.

(iii) comprise a region of at least 25, at least 30, at least 35, at least 40, at least 41, at least 42, at least 43, at least 44, at least 45, at least 46, or the full-length of 47 contiguous bases from SEQ ID NO: 26, or a region of at least 25, at least 30, at least 35, at least 40, at least 41, at least 42, at least 43, at least 44, at least 45, at least 46, at least 47, or the full-length of 48 contiguous bases from SEQ ID NO: 27 or from a nucleic acid sequence having at least 80% sequence identity to the full-length of the nucleic acid sequence of SEQ ID NO: 26 or 27 (as defined herein) or a nucleic acid which is complementary to SEQ ID NO: 26 or 27 or a nucleic acid having at least 80% sequence identity to the full-length of the nucleic acid sequence of SEQ ID NO: 26 or 27. Said oligonucleotides hybridise to a nucleic acid sequence within a target region of the VIM gene comprises bases 190 to 270, preferably 210 to 270, even more preferably 210 to 265 of the VIM gene. Typically, this oligonucleotide specific for the VIM gene is a probe.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the forward and/or reverse primers are used, more preferably the forward and reverse primers. In an even more preferred embodiment, all three VIM specific oligonucleotides (i), (ii) and (iii) may be used according to the present invention.

The oligonucleotide(s) specific for the VIM gene may be used in combination with any of the other oligonucleotides disclosed herein, including other oligonucleotides specific for the VIM gene, and/or oligonucleotides specific for one or more other carbapenemase genes, including but not limited to an OXA-48-like gene, a KPC gene, an NDM gene, an IMP gene, an IMI gene, a GES gene and/or an SPM gene.

One or more additional oligonucleotide specific for the VIM gene may be used to determine the presence and/or amount of the VIM gene in addition to the specific oligonucleotide defined above. Said one or more additional oligonucleotide specific for the VIM gene may comprise any other oligonucleotide specific for an VIM gene as described herein. Said one or more additional oligonucleotide specific for the VIM gene may be used in any combination with any of the oligonucleotides specific for any other carbapenemase gene (examples of other carbapenemase genes and oligonucleotides specific for said other carbapenemase genes are described herein), particularly the oligonucleotides specific for the OXA-48-like and/or NDM and/or KPC and/or IMP and/or IMI carbapenemase genes as described herein.

Wherein the present invention relates to determining the presence and/or amount of a KPC gene, the at least one oligonucleotide specific for the KPC gene may comprise or consist of:

(i) a region of at least 24, at least 25, at least 26, at least 27, at least 28, at least 29 or the full-length of 30 contiguous bases from SEQ ID NO: 1, or from a nucleic acid sequence having at least 80% sequence identity to the full-length of the nucleic acid sequence of SEQ ID NO: 1 (as defined herein) or a nucleic acid which is complementary to SEQ ID NO: 1 or a nucleic acid having at least 80% sequence identity to the full-length of the nucleic acid sequence of SEQ ID NO: 1. Said oligonucleotides hybridise to a target region of the KPC gene comprising bases 100 to 180, preferably 120 to 180, more preferably 120 to 160 of the KPC gene. Typically, this oligonucleotide specific for the KPC gene is a forward primer.

(ii) a region of at least 24, at least 25, at least 26, at least 27, at least 28, at least 29, at least 30, at least 31, at least 32, at least 33, at least 34 or the full-length of 35 contiguous bases from SEQ ID NO: 2, or from a nucleic acid sequence having at least 80% sequence identity to the full-length of the nucleic acid sequence of SEQ ID NO: 2 (as defined herein) or a nucleic acid which is complementary to SEQ ID NO: 2 or a nucleic acid having at least 80% sequence identity to the full-length of the nucleic acid sequence of SEQ ID NO: 2. Said oligonucleotides hybridise to a target region of the KPC gene comprising bases 240 to 310, preferably 250 to 310, more preferably 260 to 310, even more preferably 270 to 310 of the KPC gene. Typically, this oligonucleotide specific for the KPC gene is a reverse primer.

(iii) comprise a region of at least 25, at least 30, at least 35, at least 40, at least 41, at least 42, at least 43, at least 44, at least 45, at least 46, at least 47, or the full-length of 48 contiguous bases from SEQ ID NO: 3, or from a nucleic acid sequence having at least 80% sequence identity to the full-length of the nucleic acid sequence of SEQ ID NO: 3 (as defined herein) or a nucleic acid which is complementary to SEQ ID NO: 3 or a nucleic acid having at least 80% sequence identity to the full-length of the nucleic acid sequence of SEQ ID NO: 3. Said oligonucleotides hybridise to a target region of the KPC gene comprising bases 160 to 270, preferably 160 to 240, more preferable 180 to 240 of the KPC gene. Typically, this oligonucleotide specific for the KPC gene is a probe.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the forward and/or reverse primers are used, more preferably the forward and reverse primers. In an even more preferred embodiment, all three KPC specific oligonucleotides (i), (ii) and (iii) may be used according to the present invention.

The oligonucleotide(s) specific for the KPC gene may be used in combination with any of the other oligonucleotides disclosed herein, including other oligonucleotides specific for the KPC gene, and/or oligonucleotides specific for one or more other carbapenemase genes, including but not limited to an OXA-48-like gene, a VIM gene, an NDM gene, an IMP gene, an IMI gene, a GES gene and/or an SPM gene.

One or more additional oligonucleotide specific for the KPC gene may be used to determine the presence and/or amount of the KPC gene in addition to the specific oligonucleotide defined above. Said one or more additional oligonucleotide specific for the KPC gene may comprise any other oligonucleotide specific for an KPC gene as described herein. Said one or more additional oligonucleotide specific for the KPC gene may be used in any combination with any of the oligonucleotides specific for any other carbapenemase gene (examples of other carbapenemase genes and oligonucleotides specific for said other carbapenemase genes are described herein), particularly the oligonucleotides specific for the OXA-48-like and/or VIM and/or NDM and/or IMP and/or IMI carbapenemase genes as described herein.

Wherein the present invention relates to determining the presence and/or amount of an NDM gene, the at least one oligonucleotide specific for the NDM gene may comprise or consist of:

(i) a region of at least 24, at least 25, at least 26, at least 27, at least 28, at least 29, at least 30, at least 31, at least 32, at least 33, at least 34 or the full-length of 35 contiguous bases from SEQ ID NO: 7, or from a nucleic acid sequence having at least 80% sequence identity to the full-length of the nucleic acid sequence of SEQ ID NO: 7 (as defined herein) or a nucleic acid which is complementary to SEQ ID NO: 7 or a nucleic acid having at least 80% sequence identity to the full-length of the nucleic acid sequence of SEQ ID NO: 7. Said oligonucleotides hybridise to a target region of the NDM gene comprising bases 250 to 350, preferably 280 to 330 of the NDM gene. Typically, this oligonucleotide specific for the NDM gene is a forward primer.

(ii) a region of at least 24, at least 25, at least 26, at least 27, at least 28, at least 29, at least 30, at least 31, or the full-length of 32 contiguous bases from SEQ ID NO: 8, or from a nucleic acid sequence having at least 80% sequence identity to the full-length of the nucleic acid sequence of SEQ ID NO: 8 (as defined herein) or a nucleic acid which is complementary to SEQ ID NO: 8 or a nucleic acid having at least 80% sequence identity to the full-length of the nucleic acid sequence of SEQ ID NO: 8. Said oligonucleotides hybridise to a target region of the NDM gene comprising bases 400 to 480, preferably 400 to 450, more preferably 410 to 450 of the NDM gene. Typically, this oligonucleotide specific for the NDM gene is a reverse primer.

(iii) comprise a region of at least 25, at least 30, at least 35, at least 40, at least 41, at least 42, at least 43, at least 44, at least 45, at least 46, at least 47, at least 48, or the full-length of 49 contiguous bases from SEQ ID NO: 9, or from a nucleic acid sequence having at least 80% sequence identity to the full-length of the nucleic acid sequence of SEQ ID NO: 9 (as defined herein) or a nucleic acid which is complementary to SEQ ID NO: 9 or a nucleic acid having at least 80% sequence identity to the full-length of the nucleic acid sequence of SEQ ID NO: 9. Said oligonucleotides hybridise to a target region of the NDM gene comprising bases 330 to 410, preferably 350 to 410, more preferably 360 to 410 of the NDM gene. Typically, this oligonucleotide specific for the NDM gene is a probe.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the forward and/or reverse primers are used, more preferably the forward and reverse primers. In an even more preferred embodiment, all three NDM specific oligonucleotides (i), (ii) and (iii) may be used according to the present invention.

The oligonucleotide(s) specific for the NDM gene may be used in combination with any of the other oligonucleotides disclosed herein, including other oligonucleotides specific for the NDM gene, and/or oligonucleotides specific for one or more other carbapenemase genes, including but not limited to an OXA-48-like gene, a VIM gene, a KPC gene, an IMP gene, an IMI gene, a GES gene and/or an SPM gene.

One or more additional oligonucleotide specific for the NDM gene may be used to determine the presence and/or amount of the NDM gene in addition to the specific oligonucleotide defined above. Said one or more additional oligonucleotide specific for the NDM gene may comprise any other oligonucleotide specific for an NDM gene as described herein. Said one or more additional oligonucleotide specific for the NDM gene may be used in any combination with any of the oligonucleotides specific for any other carbapenemase gene (examples of other carbapenemase genes and oligonucleotides specific for said other carbapenemase genes are described herein), particularly the oligonucleotides specific for the OXA-48-like and/or VIM and/or KPC and/or IMP and/or IMI carbapenemase genes as described herein.

Wherein the present invention relates to determining the presence and/or amount of an IMP gene, the at least one oligonucleotide specific for the IMP gene may comprise or consist of:

(i) a region of at least 24, at least 25, at least 26, at least 27, at least 28 or the full-length of 29 contiguous bases from SEQ ID NO: 10 or 11, or from a nucleic acid sequence having at least 80% sequence identity to the full-length of the nucleic acid sequence of SEQ ID NO: 10 or 11 (as defined herein) or a nucleic acid which is complementary to SEQ ID NO: 10 or 11 or a nucleic acid having at least 80% sequence identity to the full-length of the nucleic acid sequence of SEQ ID NO: 10 or 11. Said oligonucleotides hybridise to a target region of the IMP gene comprising bases 730 to 783, preferably 750 to 783 of the IMP gene. Typically, this oligonucleotide specific for the IMP gene is a forward primer.

(ii) a region of at least 24, at least 25, at least 26, at least 27, at least 28, at least 29 or the full-length of 30 contiguous bases from any one of SEQ ID NOs: 12 to 22, or from a nucleic acid sequence having at least 80% sequence identity to the full-length of the nucleic acid sequence of any one of SEQ ID NOs: 12 to 22 (as defined herein) or a nucleic acid which is complementary to any one of SEQ ID NOs: 12 to 22 or a nucleic acid having at least 80% sequence identity to the full-length of the nucleic acid sequence of any one of SEQ ID NOs: 12 to 22. Said oligonucleotides hybridise to a target region of the IMP gene comprising bases 850 to 920, preferably 870 to 910 and/or 850 to 886, more preferably 870 to 886 of the IMP gene. Typically, this oligonucleotide specific for the IMP gene is a reverse primer.

(iii) comprise a region of at least 25, at least 30, at least 35, at least 40, at least 41, at least 42, at least 43, at least 44, at least 45, at least 46, at least 47, at least 48, at least 49 or the full-length of 50 contiguous bases from SEQ ID NO: 23, or from a nucleic acid sequence having at least 80% sequence identity to the full-length of the nucleic acid sequence of SEQ ID NO: 23 (as defined herein) or a nucleic acid which is complementary to SEQ ID NO: 23 or a nucleic acid having at least 80% sequence identity to the full-length of the nucleic acid sequence of SEQ ID NO: 23. Said oligonucleotides hybridise to a target region of the IMP gene comprising bases 784 to 860, preferably 784 to 840 of the IMP gene. Typically, this oligonucleotide specific for the IMP gene is a probe.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the forward and/or reverse primers are used, more preferably the forward and reverse primers. In an even more preferred embodiment, all three IMP specific oligonucleotides (i), (ii) and (iii) may be used according to the present invention.

The oligonucleotide(s) specific for the IMP gene may be used in combination with any of the other oligonucleotides disclosed herein, including other oligonucleotides specific for the IMP gene, and/or oligonucleotides specific for one or more other carbapenemase genes, including but not limited to an OXA-48-like gene, a VIM gene, a KPC gene, an NDM gene, an IMI gene, a GES gene and/or an SPM gene.

One or more additional oligonucleotide specific for the IMP gene may be used to determine the presence and/or amount of the IMP gene in addition to the specific oligonucleotide defined above. Said one or more additional oligonucleotide specific for the IMP gene may comprise any other oligonucleotide specific for an IMP gene as described herein. Said one or more additional oligonucleotide specific for the IMP gene may be used in any combination with any of the oligonucleotides specific for any other carbapenemase gene (examples of other carbapenemase genes and oligonucleotides specific for said other carbapenemase genes are described herein), particularly the oligonucleotides specific for the OXA-48-like and/or VIM and/or KPC and/or NDM and/or IMI carbapenemase genes as described herein.

Wherein the present invention relates to determining the presence and/or amount of an IMI gene, the at least one oligonucleotide specific for the IMI gene may comprise or consist of:

(i) a region of at least 24, at least 25, at least 26, at least 27, at least 28 or the full-length of 29 contiguous bases from SEQ ID NO: 28, or from a nucleic acid sequence having at least 80% sequence identity to the full-length of the nucleic acid sequence of SEQ ID NO: 28 (as defined herein) or a nucleic acid which is complementary to SEQ ID NO: 28 or a nucleic acid having at least 80% sequence identity to the full-length of the nucleic acid sequence of SEQ ID NO: 28. Said oligonucleotides hybridise to a target region of the IMI gene comprising bases 410 to 446, preferably 418 to 446 of the IMI gene. Typically, this oligonucleotide specific for the IMI gene is a forward primer.

(ii) a region of at least 24, at least 25, at least 26, at least 27, at least 28, at least 29, at least 30 or the full-length of 31 contiguous bases from SEQ ID NO: 29 or a region of at least 24, at least 25, at least 26, at least 27, at least 28 or the full-length of 29 contiguous bases from SEQ ID NO: 30, or from a nucleic acid sequence having at least 80% sequence identity to the full-length of the nucleic acid sequence of SEQ ID NO: 29 or 30 as defined herein) or a nucleic acid which is complementary to SEQ ID NO: 29 or 30 or a nucleic acid having at least 80% sequence identity to the full-length of the nucleic acid sequence of SEQ ID NO: 39 or 30. Said oligonucleotides hybridise to a target region of the IMI gene comprising bases 507 to 560, preferably 507 to 550 or 517 to 560, more preferably 507 to 537 or 517 to 545 of the IMI gene. Typically, this oligonucleotide specific for the IMI gene is a reverse primer.

(iii) comprise a region of at least 25, at least 30, at least 35, at least 40, at least 41, at least 42, at least 43, at least 44, at least 45, at least 46, at least 47, at least 48, or the full length of 49 contiguous bases from SEQ ID NO: 31, or from a nucleic acid sequence having at least 80% sequence identity to the full-length of the nucleic acid sequence of SEQ ID NO: 31 (as defined herein) or a nucleic acid which is complementary to SEQ ID NO: 31 or a nucleic acid having at least 80% sequence identity to the full-length of the nucleic acid sequence of SEQ ID NO: 31. Said oligonucleotides hybridise to a target region of the IMI gene comprising bases 452 to 505, preferably 452 to 500 of the IMI gene. Typically, this oligonucleotide specific for the IMI gene is a probe.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the forward and/or reverse primers are used, more preferably the forward and reverse primers. In an even more preferred embodiment, all three IMI specific oligonucleotides (i), (ii) and (iii) may be used according to the present invention.

The oligonucleotide(s) specific for the IMI gene may be used in combination with any of the other oligonucleotides disclosed herein, including other oligonucleotides specific for the IMP gene, and/or oligonucleotides specific for one or more other carbapenemase genes, including but not limited to an OXA-48-like gene, a VIM gene, a KPC gene, an NDM gene, an IMP gene, a GES gene and/or an SPM gene.

One or more additional oligonucleotide specific for the IMI gene may be used to determine the presence and/or amount of the IMI gene in addition to the specific oligonucleotide defined above. Said one or more additional oligonucleotide specific for the IMI gene may comprise any other oligonucleotide specific for an IMI gene as described herein. Said one or more additional oligonucleotide specific for the IMI gene may be used in any combination with any of the oligonucleotides specific for any other carbapenemase gene (examples of other carbapenemase genes and oligonucleotides specific for said other carbapenemase genes are described herein), particularly the oligonucleotides specific for the OXA-48-like and/or VIM and/or KPC and/or NDM and/or IMP carbapenemase genes as described herein.

Wherein the present invention relates to determining the presence and/or amount of a CTX-M gene, the at least one oligonucleotide specific for the CTX-M gene may comprise or consist of:

(i) a region of at least 24, at least 25, at least 26, at least 27, at least 28, at least 29, at least 30, at least 31, at least 32, at least 33, at least 34 or the full-length of 35 contiguous bases from SEQ ID NO: 32 or 33, or from a nucleic acid sequence having at least 80% sequence identity to the full-length of the nucleic acid sequence of SEQ ID NO: 32 or 33 (as defined herein) or a nucleic acid which is complementary to SEQ ID NO: 32 or 33 or a nucleic acid having at least 80% sequence identity to the full-length of the nucleic acid sequence of SEQ ID NO: 32 or 33. Typically, this oligonucleotide specific for the CTX-M gene is a forward primer.

(ii) a region of at least 24, at least 25, at least 26, at least 27, at least 28, at least 29, at least 30, at least 31, at least 32, at least 33, at least 34 or the full-length of 35 contiguous bases from SEQ ID NO: 34 or 35, or from a nucleic acid sequence having at least 80% sequence identity to the full-length of the nucleic acid sequence of SEQ ID NO: 34 or 35 (as defined herein) or a nucleic acid which is complementary to SEQ ID NO: 34 or 35 or a nucleic acid having at least 80% sequence identity to the full-length of the nucleic acid sequence of SEQ ID NO: 34 or 35. Typically, this oligonucleotide specific for the CTX-M gene is a reverse primer.

(iii) comprise a region of at least 25, at least 30, at least 35, at least 40, at least 41, at least 42, at least 43, at least 44, at least 45, at least 46, or the full length of 47 contiguous bases from SEQ ID NO: 36, or a region of at least 25, at least 30, at least 35, at least 40, at least 41, at least 42, at least 43, at least 44, at least 45, at least 46, at least 47, at least 48, at least 49 or the full length of 50 contiguous bases from SEQ ID NO: 37, or from a nucleic acid sequence having at least 80% sequence identity to the full-length of the nucleic acid sequence of SEQ ID NO: 36 or 37 (as defined herein) or a nucleic acid which is complementary to SEQ ID NO: 36 or 37 or a nucleic acid having at least 80% sequence identity to the full-length of the nucleic acid sequence of SEQ ID NO: 36 or 37. Typically, this oligonucleotide specific for the CTX-M gene is a probe.

As well as being used separately, any combination of these oligonucleotides (i), (ii) and (ii) may be used in accordance with the present invention. For example, the oligonucleotides may be used in combination as forward primer/reverse primer ((i) and (ii)), forward primer/probe ((i) and (iii)), reverse primer/probe ((ii) and (iii)) or forward primer/reverse primer/probe ((i), (ii) and (iii)) as indicated above. In a preferred embodiment, the forward and/or reverse primers are used, more preferably the forward and reverse primers. In an even more preferred embodiment, all three CTX-M specific oligonucleotides (i), (ii) and (iii) may be used according to the present invention.

The oligonucleotide(s) specific for the CTX-M gene may be used in combination with any of the other oligonucleotides disclosed herein, including other oligonucleotides specific for the CTX-M gene, and/or oligonucleotides specific for one or more carbapenemase genes, including but not limited to an OXA-48-like gene, a VIM gene, a KPC gene, an NDM gene, an IMP gene, an IMI gene, a GES gene and/or an SPM gene.

One or more additional oligonucleotide specific for the CTX-M gene may be used to determine the presence and/or amount of the CTX-M gene in addition to the specific oligonucleotide defined above. Said one or more additional oligonucleotide specific for the CTX-M gene may comprise any other oligonucleotide specific for a CTX-M gene as described herein. Said one or more additional oligonucleotide specific for the CTX-M gene may be used in any combination with any of the oligonucleotides specific for any carbapenemase gene (examples of carbapenemase genes and oligonucleotides specific for said other carbapenemase genes are described herein), particularly the oligonucleotides specific for the OXA-48-like and/or VIM and/or KPC and/or NDM and/or IMP and/or IMI carbapenemase genes as described herein.

An oligonucleotide specific for the one or more carbapenemase gene, or an oligonucleotide probe or primer may comprise or be complementary or reverse complementary to a nucleic acid sequence within a target nucleic acid sequence from the one or more carbapenemase gene of the invention, or to a nucleic acid sequence having at least 80% identity to said target nucleic acid sequence. Any suitable oligonucleotide specific for the one or more carbapenemase gene, or any suitable oligonucleotide probe or primer which comprises or is complementary (as defined herein) to a nucleic acid sequence within a target nucleic acid sequence of one or more carbapenemase gene of the invention may be used.

TABLE 1
Primer and probe sequences

| Forward (SEQ ID NO) | | Reverse (SEQ ID NO) | | Probe (SEQ ID NO) | |
|---|---|---|---|---|---|
| CTX-M- | CCGAATCTGTTAAATCAGC | CTX-M- | CTAGCCGGGCCGCCAAC | CTX-M- | GATTGCGGAAAAGCACGTCAATG |
| 15_forward | GAGTTGAGATCAAAAA | 15_reverse | GTGAGCAATCAGCTTATT | 15_probe | GGACGAT⁵G⁶T⁷CACTGGCTGAGC |
| | (32) | | (34) | | TTA (36) |
| CTX-M- | CACGTCAACGGCACAATGA | CTX-M- | TCCAGACGAAACGTCTCA | CTX-M- | AGTACAGCGACAATACCGCCATG |
| 14_forward | CGCTGGCAGAACTGAG | 14_reverse | TCGCCGATCGCGCGGGC | 14_probe | AACAAATT⁵G⁶AT⁷TGCCCAGCTC |
| | (33) | | (35) | | GGTGGC (37) |
| KPC_forward | CCCATCTCGGAAAAATATC | KPC_reverse | CTCCAGCTCCCAGCGGTC | KPC_probe | CGTGCAATACAGTGATAACGCCG |
| | TGACAACAGGC (1) | | CAGACGGAACGTGGTAT | | CCGCCAAT⁵T⁶T⁷GTTGCTGAAGG |
| | | | (2) | | AGTT(3) |
| NDM_forward | GACCAGACCGCCCAGATCC | NDM_reverse | CTGGTTCGACAACGCATT | NDM_probe | CCCCGCCGCATGCAGCGCGTCCA |
| | TCAACTGGATCAAGCA | | GGCATAAGTCGCAA (8) | | TACCGCCCAT⁵C⁶T⁷TGTCCTGAT |
| | (7) | | | | GCGCG (9) |
| OXA-48- | CCTTATCGGCTGTGTTTTT | OXA-48- | CCCGTTTAAGATTATTGG | OXA-48- | TGGCAAGAAAACAAAAGTTGGAA |
| like_forward | GGTGGCATCGA (4) | like_reverse | TAAATCCTTGCTG (5) | like_probe | TGCTCACT⁵T⁶T⁷ACTGAACATAA |
| | | | | | ATCA (6) |
| VIM_forward | CCAGATTGCCGATGGTGTT | VIM_reverse | GGAAGTCCAATTTGCTTT | VIM_probe | CTCATTGTCCGTGATGGTGATGA |
| | TGGTCGCATATCGC (24) | | TCAATCTCCGCGAGAAG | | GTTGCTT⁵T⁶T⁷GATTGATACAGC |
| | | | (25) | | GTG(26) |
| | | | | VIM_probe 2 | CTTATCGTCCGCGATGCTGATGA |
| | | | | | GTTGCTT⁵C⁶T⁷TATTGATACAGC |
| | | | | | GTGG (27) |
| IMP_forward_1 | GGAATAGAGTGGCTTAATT | IMP_reverse | AAAAAACTTCAATTTTAT | IMP_probe | CCCACGTATGCATCTGAATTAAC |
| | CTCAATCTAT (10) | | TTTTAACTAGCC (12) | | AAATGAACT⁵G⁶CT⁷TAAAAAAGA |
| | | IMP_reverse_2 | AAAAAATTTCAATCTTTT | | CGGTAA (23) |
| | | | TCTTAACTAACC (13) | | |
| | | IMP_reverse_3 | AAAAAACTTCAATTTTGT | | |
| | | | TTTTAATTAGTG (14) | | |
| | | IMP_reverse_4 | AAAAAATTTCAATTTTAT | | |
| | | | TTTTAAGTAGCC (15) | | |
| | | IMP_reverse_5 | AGAAAACTTCAATTTTGT | | |
| | | | TTTTGATAAGTG (16) | | |
| IMP_forward_2 | GGAATAGAGTGGCTTAATT | IMP_reverse_6 | AAAACACTTCTATTTTAT | | |
| | CTCGATCTAT (11) | | CTTTCGCCAGCC (17) | | |
| | | IMP_reverse_7 | AAAAAACTTCAATTTTGT | | |
| | | | TTTTTATTAGTG (18) | | |
| | | IMP_reverse_8 | TTCAATTTTATTTTTAAC | | |
| | | | TAGCCAATA (19) | | |
| | | IMP_reverse_9 | TTCAATCTTTTTCTTAAC | | |
| | | | TAACCAATA (20) | | |
| | | IMP_reverse_10 | TTCAATTTTGTTTTTAAT | | |
| | | | TAGTGAATA (21) | | |
| | | IMP_reverse_11 | TTCTATTTTATCTTTCGC | | |
| | | | CAGCCAATA (22) | | |
| IMI_forward | CTTGAACGATATATCGGTG | IMI_reverse | TTCATCGCCAGGAATAGC | IMI_probe | CTAAATTCATGCGGTCGATTGGA |
| | GTCCTGAGGG (28) | | TGTGTTTAGATCT (29) | | GATAAAGATT⁵T⁶T⁷AGACTCGAT |
| | | IMI_reverse_2 | GTGTCACGTTCATCGCCA | | CGTTG (31) |
| | | | GGAATAGCTGT (30) | | |

⁵ = fluorophore preferably linked at this position
⁶ = preferably position of abasic residue
⁷ = quencher preferably linked at this position

It is preferred that the binding conditions for an oligonucleotide specific for the one or more carbapenemase gene, or an oligonucleotide probe or primer hybridising to its target sequence are such that a high level of specificity is provided—i.e. hybridisation of the oligonucleotide, probe or primer occurs under “stringent conditions”. In general, stringent conditions are selected to be about 5° C. lower than the thermal melting point (Tm) for the specific sequence at a defined ionic strength and pH. The Tm is the temperature (under defined ionic strength and pH) at which 50% of the target (or complement) sequence hybridises to a perfectly matched probe or primer. In this regard, the Tm of oligonucleotides, probes or primers of the present invention, at a salt concentration of about 0.02M or less at pH 7, is for example above 60° C., such as about 70° C.

Premixed buffer solutions are commercially available (e.g. EXPRESSHYB Hybridisation Solution from CLONTECH Laboratories, Inc.), and hybridisation can be performed according to the manufacturer's instructions.

Oligonucleotides, probes and primers of the present invention may be screened to minimise self-complementarity and dimer formation (oligonucleotide-oligonucleotide, probe-probe or primer-primer binding).

Any of the oligonucleotides, probes or primers described herein may comprise a tag and/or label. The tag and/or label may, for example, be located (independently of one another) towards the middle or towards or at the 5′ or 3′ end of the herein described oligonucleotides/probes/primers, for example at the 5′ end.

Hence, following hybridisation of tagged/labelled probe to target nucleic acid, the tag/label is associated with the target nucleic acid in the one or more carbapenemase gene or fragment thereof. Alternatively, if an amplification step is employed, the probes may act as primers during the method of the invention and the tag/label may therefore become incorporated into the amplification product as the primer is extended.

Examples of suitable labels include detectable labels such as radiolabels or fluorescent or coloured molecules, enzymatic markers or chromogenic markers—e.g. dyes that produce a visible colour change upon hybridisation of the probe or primer. By way of example, the label may be digoxygenin, fluorescein-isothiocyanate (FITC), R-phycoerythrin, Alexa 532, carboxy-X-rhodamine (ROX), carboxytetramethylrhodamine (TAMRA), 4,5-dichloro-dimethoxy-fluorescein (JOE), BHQ-1/2/3, Cy5, Cy5.5 or Cy3. The oligonucleotides, probes or primer preferably contain a Fam label (e.g. a 5′ Fam label), and/or a minor groove binder (MGB). The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

Examples of suitable tags include “complement/anti-complement pairs”. The term “complement/anti-complement pair” denotes non-identical moieties that form a non-covalently associated, stable pair under appropriate conditions. Examples of suitable tags include biotin and streptavidin (or avidin). By way of example, a biotin tag may be captured using streptavidin, which may be coated onto a substrate or support such as a bead (for example a magnetic bead) or membrane. Likewise, a streptavidin tag may be captured using biotin, which may be coated onto a substrate or support such as a bead (for example a magnetic bead) or membrane. Other exemplary complement/anti-complement pairs include receptor/ligand pairs, antibody/antigen (or hapten or epitope) pairs, and the like. Another example is a nucleic acid sequence tag that binds to a complementary sequence. The latter may itself be pre-labelled, or may be attached to a surface (e.g. a bead) which is separately labelled. An example of the latter embodiment is the well-known LuminexR bead system. Other exemplary pairs of tags and capture molecules include receptor/ligand pairs and antibody/antigen (or hapten or epitope) pairs. Where subsequent dissociation of the complement/anti-complement pair is desirable, the complement/anti-complement pair has a binding affinity of, for example, less than 10⁹ M⁻¹. One exemplary tagged oligonucleotide, probe or primer is a biotin-labelled oligonucleotide, probe or primer, which may be detected using horse-radish peroxidase conjugated streptavidin.

The oligonucleotides, probes or primers of the invention may be labelled with different labels or tags, thereby allowing separate identification of each oligonucleotide, probe or primer when used in the method of the present invention.

Any conventional method may be employed to attach nucleic acid tags to an oligonucleotide, probe or primer of the present invention (e.g. to the 5′ end of the defined binding region of the oligonucleotide, probe or primer). Alternatively, oligonucleotides, probes or primers of the invention (with pre-attached nucleic acid tags) may be constructed by commercial providers.

Detection of the one or more carbapenemase gene, a target region of the one or more carbapenemase gene or a fragment of said gene or target region may be carried out by any known means. In this regard, the probe or amplification product may be tagged and/or labelled, and the detection method may therefore comprise detecting said tag and/or label.

In one embodiment, the oligonucleotide(s), probe(s) or primer(s) may comprise a tag and/or label. Thus, in one embodiment, following hybridisation of tagged/labelled oligonucleotide/probe/primer to target nucleic acid in the one or more carbapenemase gene, the tag/label becomes associated with the target nucleic acid. Thus, in one embodiment, the assay may comprise detecting the tag/label and correlating presence of tag/label with presence of the one or more carbapenemase gene of the invention.

In one embodiment, tag and/or label may be incorporated during extension of the oligonucleotide(s), probe(s) or primer(s). In doing so, the amplification product(s) become tagged/labelled, and the assay may therefore comprise detecting the tag/label and correlating presence of tag/label with presence of amplification product, and hence the presence of one or more carbapenemase gene of the invention.

By way of example, in one embodiment, the amplification product may incorporate a tag/label (e.g. via a tagged/labelled dNTP such as biotin-dNTP) as part of the amplification process, and the assay may further comprise the use of a binding partner complementary to said tag (e.g. streptavidin) that includes a detectable tag/label (e.g. a fluorescent label, such as R-phycoerythrin). In this way, the amplified product incorporates a detectable tag/label (e.g. a fluorescent label, such as R-phycoerythrin).

In one embodiment, the oligonucleotide(s), probe(s) or primer(s) and/or the amplification product(s) may include a further tag/label (as the complement component) to allow capture of the amplification product(s).

By way of example, a “complement/anti-complement” pairing may be employed in which an anti-complement capture component binds to said further tag/label (complement component) and thereby permits capture of the probe(s) and/or amplification product(s). Examples of suitable “complement/anti-complement” partners have been described earlier in this specification, such as a complementary pair of nucleic acid sequences, a complementary antibody-antigen pair, etc. The anti-complement capture component may be attached (e.g. coated) on to a substrate or solid support—examples of suitable substrates/supports include membranes and/or beads (e.g. a magnetic or fluorescent bead). Capture methods are well known in the art. For example, LuminexR beads may be employed. Alternatively, the use of magnetic beads may be advantageous because the beads (plus captured, tagged/labelled amplification product) can easily be concentrated and separated from the sample, using conventional techniques known in the art.

Typically, a probe of the invention is typically labelled with a fluorescent marker. In some embodiments, a probe sequence of the invention is labelled with a fluorophore and quencher. The fluorophore and quencher may be covalently linked to residues (preferably T) within the sequence, separated by an abasic site. Such fluorophore/quenchers are known in the art and could be readily selected by the skilled person. Non-limiting examples of such fluorophore/quenchers include commercially available exo labels (so-called because exonuclease III (exo) is used to hydrolyse the probe to separate the fluorophore and quencher) and LF (lateral flow) labels, also known as nfo (again, so called because endonuclease IV (nfo) is used to hydrolyse the probe to separate the fluorophore and quencher). Preferred positions for linkage of the fluorophore, quencher and/or abasic residue are shown in Table 1 herein. For probes labelled using the LF system, one of the forward or reverse primers is typically labelled with biotin, as disclosed herein.

Alternatively, the fluorophore may be attached to an abasic site within the probe sequence. Again, such fluorophore are known in the art and could be readily selected by the skilled person. Non-limiting examples of such fluorophore include commercially available fpg labels (again, so called because formamidopyrimidine [fapy]-DNA glycosylase (fpg) is used to hydrolyse the probe to separate the fluorophore and quencher). In such cases, the quencher is typically located 5′ to the fluorophore, preferably at the 5′ end of the oligonucleotide.

Typically a probe sequence of the invention further comprises a 3′ blocking group, including, but not limited to a 3′ propanol blocker.

Immobilisation provides a physical location for the oligonucleotides, probes, primers and/or anti-complement capture component of the invention, and may serve to fix the capture component/oligonucleotide/probe/primer at a desired location and/or facilitate recovery or separation of oligonucleotide/probe/primer. The support may be a rigid solid support made from, for example, glass, plastic or silica, such as a bead (for example a fluorescent or magnetic bead). Alternatively, the support may be a membrane, such as nylon or nitrocellulose membrane. 3D matrices are also suitable supports for use with the present invention—e.g. polyacrylamide or PEG gels. Immobilisation to a support/platform may be achieved by a variety of conventional means. By way of example, immobilisation onto a support such as a nylon membrane may be achieved by UV cross-linking. Alternatively, biotin-labelled molecules may be bound to streptavidin-coated substrates (and vice-versa), and molecules prepared with amino linkers may be immobilised on to silanised surfaces. Another means of immobilisation is via a poly-T tail or a poly-C tail, for example at the 3′ or 5′ end. Said immobilisation techniques apply equally to the probe component (and primer/primer pair component, if present) of the present invention.

In one embodiment, the oligonucleotide, probes and/or primers of the invention comprise a nucleic acid sequence tag/label (e.g. attached to each probe at the 5′ end of the defined sequence of the probe/primer that binds to target/complement nucleic acid). In more detail, each of the oligonucleotides/probes/primers is provided with a different nucleic acid sequence tag/label, wherein each of said tags/labels (specifically) binds to a complementary nucleic acid sequence present on the surface of a bead. Each of the different tags/labels binds to its complementary sequence counterpart (and not to any of the complementary sequence counterparts of the other tags), which is located on a uniquely identifiable bead. In this regard, the beads are uniquely identifiable, for example by means of fluorescence at a specific wavelength. Thus, in use, oligonucleotides/probes/primers of the invention bind to target nucleic acid (if present in the sample). Thereafter, (only) the bound probes may be extended (in the 3′ direction) in the presence of one or more labelled dNTP (e.g. biotin labelled dNTPs, such as biotin-dCTPs).

The extended primers may be contacted with a binding partner counterpart to the labelled dNTPs (e.g. a streptavidin labelled fluorophore, such as streptavidin labelled R-phycoerythrin), which binds to those labelled dNTPs that have become incorporated into the extended primers. Thereafter, the labelled extended primers may be identified by allowing them to bind to their nucleic acid counterparts present on the uniquely identifiable beads. The latter may then be “called” (e.g. to determine the type of bead present by wavelength emission) and the nature of the primer extension (and thus the type of target/complement nucleic acid present) may be determined.

Typically, probes/primers of the invention are oligonucleotides having sequence identity or complementarity with a region of the one or more carbapenemase gene (either the sense strand, the complementary strand or the reverse of either strand) of the invention as disclosed herein. One or more probe may be immobilised on a solid support, and used to interrogate mRNA or DNA obtained from a test sample. If the mRNA or DNA from the test sample contains the one or more carbapenemase gene targeted by the immobilised probe, it will bind to the probe, and may then be detected. The probes/primers of the invention may also be detected using PCR, such as real time PCR.

Any oligonucleotide with the appropriate level of sequence identity with the one or more carbapenemase gene of the invention, or with one or more target sequences within said one or more carbapenemase gene of the invention may be used as a probe or primer as described herein. Any oligonucleotide with the appropriate level of complementarity with the one or more carbapenemase gene of the invention, or with one or more target sequences within said one or more carbapenemase gene of the invention may be used as a probe or primer as described herein.

### Methods of Diagnosis

The methods of the invention have clinical value in providing a “rule out” assay to inform prescribing practice. To achieve this aim, the methods described herein are typically used in combination, to provide an overall assessment of the presence of carbapenemase genes likely to result in treatment failure. The output of the methods would be a single (positive or negative) result for the presence of one or more of the carbapenemase genes. A carbapenemase-positive result would mean that a clinician could not safely treat with carbapenem antibiotics (e.g. meropenem, imipenem) and should select an alternative treatment option. A carbapenemase-negative result would suggest that there is a much greater chance of a treatment with a carbapenem being effective and would encourage the use of this antibiotic if other symptoms and clinical setting were appropriate.

Accordingly, as described herein, the present invention provides a method for diagnosing a presumptive carbapenemase-producing bacterial infection in an individual, comprising determining the presence and/or amount of one or more carbapenemase-producing bacteria in a sample obtained from the individual by determining the presence and/or amount of a carbapenemase gene selected from one or more OXA-48-like gene, one or more VIM gene, one or more NDM gene, one or more IMP gene, one or more IMI gene and/or one or more KPC gene in said sample.

The presence and/or amount of combination of these carbapenemases may be detected according to the present invention in order to diagnose a carbapenemase-producing bacterial infection. For example, the invention may relate to the detection of any two (for example: VIM and NDM; VIM and IMP; VIM and KPC; VIM and OXA-48-like; VIM and IMI; NDM and IMP; NDM and KPC; NDM and OXA-48-like; NDM and IMI; IMP and KPC; IMP and OXA-48-like; IMP and IMI; KPC and OXA-48-like; KPC and IMI; or OXA-48-like and IMI), any three (for example: VIM, NDM and IMP; VIM, NDM and KPC; VIM, NDM and OXA-48-like; VIM, NDM and IMI; VIM, IMP and KPC; VIM, IMP and OXA-48-like; VIM, IMP and IMI; VIM, KPC and OXA-48-like; VIM, KPC and IMI; VIM, OXA-48-lie and IMI; NDM, IMP and KPC; NDM, IMP and OXA-48-like; NDM, IMP and IMI; NDM, KPC and OXA-48-like; NDM, KPC and IMI; NDM, OXA-48-like and IMI; IMP, KPC and OXA-48-like; IMP, KPC and IMI; IMP, OXA-48-like and IMI; or KPC, OXA-48-like and IMI), any four (for example: VIM, NDM, IMP and KPC; VIM, NDM, IMP and OXA-48-like; VIM, NDM, IMP and IMI; VIM, IMP, KPC and OXA-48-like; VIM, NDM, KPC and OXA-48-like; or NDM, IMP, KPC and OXA-48-like; VIM, NDM, OXA-48-like and IMI; VIM, IMP, OXA-48-like and IMI; VIM, KPC, OXA-48-like and IMI; NDM, IMP, OXA-48-like and IMI; NDM, KPC, OXA-48-like and IMI; IMP, KPC, OXA-48-like and IMI; VIM, IMP, KPC and IMI; VIM, NDM, KPC and IMI; or NDM, IMP, KPC and IMI), any five of the carbapenemase genes (for example: VIM, NDM, IMP, KPC and OXA-48-like; VIM, NDM, IMI, KPC and OXA-48-like; IMI, NDM, IMP, KPC and OXA-48-like; VIM, NDM, IMP, KPC and IMI; VIM, IMP, KPC, OXA-48-like and IMI; or VIM, NDM, IMP, OXA-48-like and IMI), or all six of the carbapenemase genes (VIM, NDM, IMP, KPC, IMI and OXA-48-like).

The presence and/or amount of one or more additional carbapenemase gene in said sample may also be determined. In particular, the presence and/or amount of one or more of the additional carbapenemases disclosed herein may be determined in a diagnostic method according to the present invention.

The one or more additional carbapenemase is not limited, and may be any carbapenemase as disclosed herein, or any combination of said additional carbapenemases. For example, the present invention relates to a method for detecting the presence and/or amount of one or more carbapenemase-producing bacteria (carbapenem-resistant bacteria) comprising determining the presence and/or amount of one or more OXA-48-like gene, one or more VIM gene, one or more NDM gene, one or more IMP gene, one or more IMI gene, or one or more KPC gene, or of any combination thereof, wherein the presence and/or amount of at least one of a GES carbapenemase, an SME carbapenemase, an NmcA carbapenemase, a CphA carbapenemase, a CMY carbapenemase, an OXA carbapenemase, a SPM carbapenemase, an AIM carbapenemase, a GIM carbapenemase, a DIM carbapenemase and/or a CcrA carbapenemase is also determined. Said method may involve the detection of at least two, at least three, at least four, at least five, at least six, at least seven, at least eight, at least nine, at least ten, at least 11, or all 12 of an a GES carbapenemase, an SME carbapenemase, an NmcA carbapenemase, a CphA carbapenemase, a CMY carbapenemase, an OXA carbapenemase, a SPM carbapenemase, an AIM carbapenemase, a GIM carbapenemase, a DIM carbapenemase and/or a CcrA carbapenemase or any combination thereof (in addition to any of the combinations of an OXA-48-like carbapenemase, a VIM carbapenemase, a KPC carbapenemase, an IMI carbapenemase, an IMP carbapenemase and a NDM carbapenemase) as disclosed herein.

As a non-limiting example, the presence and/or amount of at least one of an SME carbapenemase gene, an SPM carbapenemase gene and/or a GES carbapenemase gene is determined in addition to the presence and/or amount of one or more OXA-48-like gene, one or more VIM gene, one or more NDM gene, one or more IMP gene one or more IMI gene and/or one or more KPC gene or combination thereof. As disclosed herein, the presence and/or amount of at least one, at least two or all three of an SME carbapenemase gene, an SPM carbapenemase gene and/or a GES carbapenemase gene, or any combination thereof may be determined in a diagnostic method according to the present invention.

The presence and/or amount of one or more CTX-M β lactamase may be determined in addition to the presence and/or amount of any combination of carbapenemase genes and additional carbapenemase genes as disclosed herein.

In a preferred embodiment, the presence and/or amount of any one of six carbapenemase genes (VIM, NDM, IMP, KPC, IMI and OXA-48-like) is determined in a diagnostic method according to the present invention. In some preferred embodiments, the presence and/or amount of VIM, NDM, IMP, KPC, IMI and OXA-48-like carbapenemase genes are determined, and optionally also the presence and/or amount of one or more CTX-M β lactamase.

The method may comprise determining the presence and/or amount of said carbapenemase gene(s) in a first sample taken from the individual at a single initial point in time and multiple time points thereafter to monitor the efficacy of treatment and disease resolution, and comparing the presence and/or amount of said carbapenemase gene(s) in said first sample to the presence and/or amount of said carbapenemase gene(s) in a reference or control sample. Said comparison may determine the status of carbapenem-resistant bacterial infection in the individual with an accuracy, sensitivity and/or specificity of at least about 99%, at least about 98%, at least about 97%, at least about 96%, at least about 95%, at least about 90%, at least about 80%, at least about 70% or at least about 60%. Typically the accuracy, sensitivity and/or specificity is of at least about 80% or at least about 90%.

The method may comprise determining the presence and/or amount of the one or more carbapenemase gene in a first sample from the individual; and comparing the presence or amount of the one or more carbapenemase gene in the individual's first sample to the presence and/or amount of the one or more carbapenemase gene in a sample from a reference or control population, said comparison being capable of classifying the individual as belonging to or not belonging to the reference or control population, wherein the comparison determines the status of carbapenem-resistant bacterial infection in the individual.

The method may further comprise determining the presence and/or amount of the one or more carbapenemase genes in a second sample taken from the individual; and comparing the presence and/or amount of the one or more carbapenemase genes in the individual's second sample to the presence and/or amount of the one or more carbapenemase genes in the control or reference sample, wherein the second comparison is capable of classifying the individual as belonging to or not belonging to the control or reference population, and wherein the second comparison determines the status of carbapenem-resistant bacterial infection in the individual.

The methods of the invention may be repeated at least once, at least twice, at least three times, at least four times, at least five times, or more. The presence and/or amount of the one or more carbapenemase genes can be determined in a separate sample taken from the individual each time the method is repeated.

The methods of the invention may be used to diagnose, detect and/or predict carbapenem-resistant bacterial infection and/or infection with one or more carbapenemase-producing bacteria. The methods of the invention may be used to distinguish between a carbapenem-resistant bacterial infection and the absence of such an infection. The methods of the invention may be used to identify an individual with a carbapenem-resistant bacterial infection and/or an individual uninfected with one or more carbapenem-resistant bacteria.

The methods of the invention may also be used to determine the status of a carbapenem-resistant bacterial infection. Determining the status of a carbapenem-resistant bacterial infection in an individual may comprise determining the progression or resolution of a carbapenem-resistant bacterial infection. Determining the status of a carbapenem-resistant bacterial infection in an individual may comprise determining the presence of a carbapenem-resistant bacterial infection in an individual.

The methods of the invention may comprise the use of a positive amplification control to ensure that the results are valid, and/or appropriate negative controls to ensure no contamination of the sample(s). Selection of suitable positive and/or negative controls is routine and within the skill of one of ordinary skill in the art.

A carbapenem-resistant bacteria infection may be diagnosed or predicted prior to the onset of clinical symptoms, and/or as subsequent confirmation after the onset of clinical symptoms. Accordingly, the present invention allows for more effective therapeutic intervention and/or diagnosis in the pre-symptomatic stage of infection.

The invention also provides the use of one or more carbapenemase gene, oligonucleotide, probe and/or primer as defined herein in the manufacture of a diagnostic for a carbapenem-resistant bacterial infection. Said diagnostic may be for diagnosing a carbapenem-resistant bacterial infection.

### Kits and Devices

The invention also provides kits and devices that are useful in determining the presence and/or amount of one or more carbapenemase-producing bacteria, the status of a carbapenem-resistant bacterial infection, and/or diagnosing or detecting a carbapenem-resistant bacterial infection. The kits and devices of the present invention comprise at least one oligonucleotide, probe and/or primer of the invention and/or one or more agent for the detection of or for the determination of the amount of the one or more carbapenemase gene of the invention, or any combination thereof as described herein. Specific oligonucleotides, probes and/or primers and agents for the detection of said one or more carbapenemase gene useful in the present invention are set forth herein. The oligonucleotides, probes and/or primers of the kit or device can be used to determine the presence and/or amount of one or more carbapenemase-producing bacteria (by determining the presence and/or amount of one or more carbapenemase gene) according to the present invention. Examples of devices of the invention include microfluidic devices, preferably designed so that detection of each carbapenemase gene (and where present, each additional gene, such as a CTX-M β lactamase) is present as a separate assay within the device.

Generally, the oligonucleotides, probes and/or primers of the kit will bind, with at least some specificity, to the one or more carbapenemase gene contained in the sample obtained from the individual. The oligonucleotides, probes and/or primers and/or agent(s) for the detection of the one or more carbapenemase gene may be part of an array, or the oligonucleotides, probes and/or primers and/or agent(s) may be packaged separately and/or individually. The oligonucleotides, probes and/or primers and/or agent(s) may be immobilised on an inert support.

The kit or device may also comprise at least one internal standard to be used in generating profiles of the one or more carbapenemase gene according to the present invention. Likewise, the internal standards can be any of the classes of compounds described above.

The kits and devices of the present invention also may contain reagents that can be used to detectably label the one or more carbapenemase gene contained in the biological samples from which the profiles of the one or more carbapenemase gene are generated. For this purpose, the kit or device may comprise antibodies which bind to the oligonucleotides, probes and/or primers of the invention. The antibodies themselves may be detectably labelled. The kit or device also may comprise a specific binding component, such as an aptamer.

In a preferred embodiment, a kit or device of the invention comprises one or more oligonucleotide, probe and/or primer specific for the one or more carbapenemase gene. In a more preferred embodiment, the one or more oligonucleotide, probe or primer specific for the one or more carbapenemase gene is an oligonucleotide of the invention, more preferably one or more of SEQ ID NOs: 1 to 31. Typically, the kit or device of the invention comprises at least one of SEQ ID NOs: 1 to 3, at least one of SEQ ID NOs: 4 to 6, at least one of SEQ ID NOs: 7 to 9, at least one of SEQ ID NOs: 10 to 23, at least one of SEQ ID NOs: 24 to 27 and/or at least one of SEQ ID NOs: 28 to 31. In a preferred embodiment, the kit or device of the invention comprises SEQ ID NOs: 1 and 2; SEQ ID NOs: 4 and 5; SEQ ID NOs: 7 and 8; SEQ ID NO: 10 and/or 11 and at least one of SEQ ID NOs: 12 to 22; SEQ ID NOs: 24 and 25; and SEQ ID NO: 28 and at least one of SEQ ID NOs: 29 and 30. In a more preferred embodiment, typically for use in methods involving RPA, the kit or device of the invention comprises SEQ ID NOs: 1 to 3; SEQ ID NOs: 4 to 6; SEQ ID NOs: 7 to 9; SEQ ID NO: 10 and/or 11, at least one of SEQ ID NOs: 12 to 22 and SEQ ID NO: 23; SEQ ID NOs: 24, 25 and at least one of SEQ ID NOs: 26 and 27; and SEQ ID NO: 28, at least one of SEQ ID NOs: 29 and 30, and SEQ ID NO: 31, or oligonucleotides with at least 80% sequence identity to said SEQ ID Nos (as defined herein). The kit or device of the invention may comprise oligonucleotides having a defined level of sequence identity with any one of the above mentioned sequence, as defined herein.

The kit or device may provide one or more oligonucleotide probe that is capable of forming a duplex with the one or more carbapenemase gene or with a complementary strand of said one or more one or more carbapenemase gene. The one or more oligonucleotide probe may be detectably labelled. Typically, the one or more oligonucleotide probe used in the methods of the invention is selected from one or more of the oligonucleotide described herein. In a preferred embodiment, the one or more oligonucleotide probe is selected from an oligonucleotide probe that comprises or is complementary to at least one nucleic acid sequence having at least 80% sequence identity to the nucleic acid sequence of any one or more of SEQ ID NOs: 1 to 31. Typically, to at least one of SEQ ID NOs: 1 to 3, at least one of SEQ ID NOs: 4 to 6, at least one of SEQ ID NOs: 7 to 9, at least one of SEQ ID NOs: 10 to 23, at least one of SEQ ID NOs: 24 to 27 and/or at least one of SEQ ID NOs: 28 to 31.

The kit or device of the present invention may further comprise one or more additional oligonucleotide, probe and/or primer specific for the one or more carbapenemase gene of the invention.

The kit or device of the present invention may further comprise one or more oligonucleotide, probe and/or primer specific for the one or more additional carbapenemase gene of the invention, or for a CTX-M β lactamase. In the latter case, the one or more oligonucleotide is preferably one as defined herein, e.g. at least one of SEQ ID NOs: 32 to 37, more preferably SEQ ID NOs: 32 and 34 and optionally SEQ ID NO: 36, or SEQ ID NOs: 33 and 35 and optionally SEQ ID NO: 37. Preferably SEQ ID NOs: 32 to 35, optionally with SEQ ID NO: 36 and/or 37 are used.

The kits and devices of the present invention may also include other classes of compounds including, but not limited to, proteins (including antibodies), and fragments thereof, peptides, polypeptides, proteoglycans, glycoproteins, lipoproteins, carbohydrates, lipids, nucleic acids, organic and inorganic chemicals, and natural and synthetic polymers. The kits and devices of the present invention may also include pharmaceutical excipients, diluents and/or adjuvants. Examples of pharmaceutical adjuvants include, but are not limited to, preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

### Sequence Information

A non-exhaustive list of OXA-48-like carbapenemase gene of the inventions is set out below. Thus, an OXA-48-like carbapenemase gene of the invention may have a polynucleotide sequence of any one of the accession numbers or SEQ ID NOs below, or a fragment or variant of one of said sequences, as defined herein.

| Genbank Accession | | |
|---|---|---|
| Number | Version Number | SEQ ID NO: |
| CP000469 | CP000469.1 GI:117610791 | 38 |
| CP000446 | CP000446.1 GI:113883030 | 39 |
| CP000444 | CP000444.1 GI:113886955 | 40 |
| JQ996150 | JQ996150.1 GI:440685600 | 41 |
| JN205800 | JN205800.1 GI:347602110 | 42 |
| HM992946 | HM992946.1 GI:304368222 | 43 |
| JX423831 | JX423831.1 GI:444236140 | 44 |
| JX893517 | JX893517.1 GI:442769982 | 45 |
| HQ700343 | HQ700343.1 GI:323817046 | 46 |
| KC902850 | KC902850.1 GI:529273301 | 47 |
| KC902852 | KC902852.1 GI:529273309 | 48 |
| JQ809466 | JQ809466.1 GI:408795221 | 49 |
| JN704570 | JN704570.1 GI:379139226 | 50 |
| KF900153 | KF900153.1 GI:573006828 | 51 |
| JX438001 | JX438001.1 GI:442577759 | 52 |
| GU197550 | GU197550.1 GI:270312218 | 53 |
| HM015773 | HM015773.1 GI:295684221 | 54 |
| AY236073 | AY236073.2 GI:69209772 | 55 |
| JX423832 | JX423832.1 GI:443909556 | 56 |
| HE617182 | HE617182.1 GI:379131236 | 57 |
| JN714122 | JN714122.1 GI:359359955 | 58 |
| JN626286 | JN626286.1 GI:359422634 | 59 |
| KC354801 | KC354801.1 GI:479327067 | 60 |
| KC757416 | KC757416.2 GI:513046067 | 61 |
| KC335143 | KC335143.1 GI:544600294 | 62 |
| KC757417 | KC757417.2 GI:513046066 | 63 |
| JX438000 | JX438000.1 GI:442577757 | 64 |

A non-exhaustive list of VIM carbapenemase gene of the inventions is set out below. Thus, a VIM carbapenemase gene of the invention may have a polynucleotide sequence of any one of the accession numbers or SEQ ID Nos below, or a fragment or variant of one of said sequences, as defined herein.

| Genbank Accession | | |
|---|---|---|
| Number | Version Number | SEQ ID NO: |
| AJ536835 | AJ536835.1 GI:30014071 | 65 |
| DQ365886 | DQ365886.1 GI:86279732 | 66 |
| AM778091 | AM778091.1 GI:157310266 | 67 |
| AY524987 | AY524987.1 GI:42406212 | 68 |
| EU090799 | EU090799.1 GI:156628070 | 69 |
| AF300454 | AF300454.1 GI:11037698 | 70 |
| DQ835309 | DQ835309.1 GI:112785289 | 71 |
| AY605049 | AY605049.2 GI:54887317 | 72 |
| DQ022682 | DQ022682.1 GI:66734335 | 73 |
| JN982330 | JN982330.1 GI:388253361 | 74 |
| GQ414736 | GQ414736.1 GI:256692864 | 75 |
| EU118148 | EU118148.2 GI:208879769 | 76 |
| EU419745 | EU419745.1 GI:171705293 | 77 |
| EU419746 | EU419746.1 GI:171705295 | 78 |
| GQ242167 | GQ242167.1 GI:253684194 | 79 |
| AY524988 | AY524988.1 GI:42406214 | 80 |
| AY524989 | AY524989.1 GI:42406216 | 81 |
| EF522838 | EF522838.1 GI:145321079 | 82 |
| AY165025 | AY165025.1 GI:26892103 | 83 |
| HM855205 | HM855205.1 GI:302340960 | 84 |
| JX982635 | JX982635.1 GI:441494187 | 85 |
| JN129451 | JN129451.1 GI:355526407 | 86 |
| AF191564 | AF191564.1 GI:7381447 | 87 |
| AJ586617 | AJ586617.2 GI:46559085 | 88 |
| KC469971 | KC469971.2 GI:465214873 | 89 |
| AY144612 | AY144612.1 GI:24415594 | 90 |
| DQ143913 | DQ143913.1 GI:72536051 | 91 |
| JX013656 | JX013656.1 GI:396586112 | 92 |
| JX982636 | JX982636.1 GI:441494189 | 93 |
| HQ858608 | HQ858608.1 GI:323473327 | 94 |
| JF803952 | JF803952.1 GI:336327549 | 95 |
| FR748153 | FR748153.1 GI:315064910 | 96 |
| FJ445404 | FJ445404.1 GI:219872982 | 97 |
| AY635904 | AY635904.1 GI:49035768 | 98 |
| FJ499397 | FJ499397.1 GI:237688234 | 99 |
| FJ822963 | FJ822963.1 GI:262180929 | 100 |
| JF900599 | JF900599.1 GI:336169750 | 101 |
| EU581706 | EU581706.1 GI:187960474 | 102 |
| EF693948 | EF693948.1 GI:157411581 | 103 |
| AY135661 | AY135661.1 GI:22758830 | 104 |
| JX311308 | JX311308.1 GI:401716858 | 105 |
| JX258134 | JX258134.1 GI:401713038 | 106 |
| JX982634 | JX982634.1 GI:441494185 | 107 |
| JN676230 | JN676230.2 GI:389656440 | 108 |
| GU724872 | GU724872.1 GI:291586255 | 109 |
| Y18050 | Y18050.2 GI:30840267 | 110 |
| HM750249 | HM750249.1 GI:308746072 | 111 |
| DQ023222 | DQ023222.1 GI:66277416 | 112 |
| FJ915116 | FJ915116.1 GI:229560242 | 113 |
| AY456196 | AY456196.1 GI:38456063 | 114 |

A non-exhaustive list of KPC carbapenemase gene of the inventions is set out below. Thus, a KPC carbapenemase gene of the invention may have a polynucleotide sequence of any one of the accession numbers or SEQ ID Nos below, or a fragment or variant of one of said sequences, as defined herein.

| Accession Number | Version Number | SEQ ID NO: |
|---|---|---|
| JF431928 | JF431928.1 GI:343952330 | 115 |
| KC433553 | KC433553.1 GI:451824560 | 116 |
| JX524191 | JX524191.1 GI:409974861 | 117 |
| KC465199 | KC465199.1 GI:478437782 | 18 |
| HQ641421 | HQ641421.1 GI:326467060 | 119 |
| AF297554 | AF297554.1 GI:10121874 | 120 |
| KC465200 | KC465200.1 GI:478437784 | 121 |
| AY034847 | AY034847.1 GI:14626419 | 122 |
| AY700571 | AY700571.1 GI:51557251 | 123 |
| HM066995 | HM066995.1 GI:296939581 | 124 |
| EU400222 | EU400222.2 GI:214010993 | 125 |
| HQ342890 | HQ342890.1 GI:327397926 | 126 |
| HQ342889 | HQ342889.1 GI:327397924 | 127 |
| FJ624872 | FJ624872.1 GI:223558116 | 128 |
| EU555534 | EU555534.1 GI:172054654 | 129 |
| GQ140348 | GQ140348.1 GI:239925270 | 130 |
| FJ234412 | FJ234412.1 GI:209892783 | 131 |
| HM769262 | HM769262.1 GI:312623594 | 132 |
| AF395881 | AF395881.1 GI:15705412 | 133 |
| EU729727 | EU729727.1 GI:189916550 | 134 |

A non-exhaustive list of NDM carbapenemase gene of the inventions is set out below. Thus, an NDM carbapenemase gene of the invention may have a polynucleotide sequence of any one of the accession numbers or SEQ ID Nos below, or a fragment or variant of one of said sequences, as defined herein.

| Accession Number | Version Number | SEQ ID NO: |
|---|---|---|
| KF361506 | KF361506.1 GI:530540024 | 135 |
| AB744718 | AB744718.1 GI:451810087 | 136 |
| JX262694 | JX262694.1 GI:401884433 | 137 |
| JN104597 | JN104597.1 GI:344226673 | 138 |
| JQ348841 | JQ348841.1 GI:378405410 | 139 |
| KC999080 | KC999080.2 GI:672816753 | 140 |
| JQ734687 | JQ734687.1 GI:388890592 | 141 |
| JN967644 | JN967644.1 GI:366085572 | 142 |
| FN396876 | FN396876.1 GI:255031061 | 143 |
| JF703135 | JF703135.1 GI:327244638 | 144 |

A non-exhaustive list of IMP carbapenemase gene of the inventions is set out below. Thus, an IMP carbapenemase gene of the invention may have a polynucleotide sequence of any one of the accession numbers or SEQ ID Nos below, or a fragment or variant of one of said sequences, as defined herein.

| Accession Number | Version Number | SEQ ID NO: |
|---|---|---|
| FN556190 | FN556190.1 GI:308026240 | 145 |
| HM138673 | HM138673.1 GI:299780310 | 146 |
| EF184215 | EF184215.1 GI:134035513 | 147 |
| AY780674 | AY780674.2 GI:76008227 | 148 |
| KF148593 | KF148593.1 GI:529283985 | 149 |
| JQ002629 | JQ002629.1 GI:403309640 | 150 |
| GQ302618 | GQ302618.2 GI:268627877 | 151 |
| GQ302617 | GQ302617.2 GI:268627875 | 152 |
| AY553332 | AY553332.1 GI:62736339 | 153 |
| AB204557 | AB204557.1 GI:59891390 | 154 |
| AB074436 | AB074436.1 GI:17026025 | 155 |
| AB777501 | AB777501.1 GI:459978576 | 156 |
| AB753458 | AB753458.1 GI:408535170 | 157 |
| DQ361087 | DQ361087.2 GI:126574581 | 158 |
| AJ584652 | AJ584652.2 GI:37653301 | 159 |
| AY033653 | AY033653.3 GI:194359396 | 160 |
| HM175876 | HM175876.1 GI:300391789 | 161 |
| EU588392 | EU588392.2 GI:194245456 | 162 |
| HQ438058 | HQ438058.1 GI:314912176 | 163 |
| EF184216 | EF184216.1 GI:134035518 | 164 |
| AY553333 | AY553333.1 GI:62736340 | 165 |
| JF810083 | JF810083.1 GI:342161745 | 166 |
| AF290912 | AF290912.1 GI:13491138 | 167 |
| JQ407409 | JQ407409.1 GI:383216584 | 168 |
| GQ221781 | GQ221781.1 GI:253987477 | 169 |
| AB777500 | AB777500.1 GI:459978567 | 170 |
| AF318077 | AF318077.1 GI:13094933 | 171 |
| JF894248 | JF894248.1 GI:335345650 | 172 |
| DQ307573 | DQ307573.1 GI:83583500 | 173 |
| GQ221782 | GQ221782.1 GI:253987479 | 174 |
| GU045307 | GU045307.1 GI:261864919 | 175 |
| HQ875573 | HQ875573.1 GI:345128596 | 176 |
| FJ384365 | FJ384365.1 GI:217038355 | 177 |
| AF288045 | AF288045.2 GI:195977007 | 178 |
| AF244145 | AF244145.1 GI:13128916 | 179 |
| AJ420864 | AJ420864.1 GI:25278055 | 180 |
| AJ243491 | AJ243491.1 GI:8546572 | 181 |
| EF192154 | EF192154.1 GI:122934823 | 182 |
| AB196988 | AB196988.1 GI:56783616 | 183 |
| EF118171 | EF118171.1 GI:122891986 | 184 |
| EU042136 | EU042136.1 GI:154867189 | 185 |
| AF322577 | AF322577.2 GI:14161935 | 186 |
| JN848782 | JN848782.2 GI:536749749 | 187 |
| JX131372 | JX131372.1 GI:401668653 | 188 |
| GU207399 | GU207399.1 GI:281021955 | 189 |
| AJ550807 | AJ550807.1 GI:33086392 | 190 |
| AJ512502 | AJ512502.1 GI:40644310 | 191 |
| AB715422 | AB715422.2 GI:697287983 | 192 |
| AB010417 | AB010417.1 GI:4760642 | 193 |
| AM283490 | AM283490.1 GI:110350566 | 194 |
| EU541448 | EU541448.1 GI:171188393 | 195 |
| AB753456 | AB753456.1 GI:408535166 | 196 |
| AB753457 | AB753457.1 GI:408535168 | 197 |
| AB195637 | AB195637.1 GI:57158242 | 198 |
| AB074433 | AB074433.1 GI:17026019 | 199 |
| AB188812 | AB188812.1 GI:51555859 | 200 |
| AB040994 | AB040994.1 GI:10566736 | 201 |
| DQ522237 | DQ522237.1 GI:102231664 | 202 |
| AB469046 | AB469046.1 GI:211922742 | 203 |
| S71932 | S71932.1 GI:560551 | 204 |
| JF816544 | JF816544.1 GI:350285632 | 205 |

A non-exhaustive list of IMI carbapenemase gene of the inventions is set out below. Thus, an IMI carbapenemase gene of the invention may have a polynucleotide sequence of any one of the accession numbers or SEQ ID NOs below, or a fragment or variant of one of said sequences, as defined herein.

| Accession Number | Version Number | SEQ ID NO: |
|---|---|---|
| U50278 | U50278.1 GI:1245353 | 206 |
| DQ173429 | DQ173429.1 GI:74229921 | 207 |
| GU015024 | GU015024.1 GI:261349302 | 208 |
| KF958750 | KF958750.1 GI:570348258 | 209 |
| KM103296 | KM103296.1 GI:692334119 | 210 |
| KP081315 | KP081315.1 GI:728678245 | 211 |

The following Examples illustrate the invention.

### EXAMPLES

### Example 1: Design and Generation of Primers and Probes; Performance of the Assay as a “Monoplex” Assay for Multiple Targets, Dose Response Curves and Determination of LoD

Extended spectrum beta lactamase (ESBL) and carbapenemase gene DNA sequences were identified from public databases and DNA sequence alignments generated.

Following analysis of the DNA sequence alignments, primer and probe sequences were designed to maximise coverage of the different gene variants. Newly designed primers and probes for KPC, NDM, OXA, VIM, IMP, IMI, CTX-M-15 and CTX-M-14 are shown in Table 2 below.

TABLE 2
Primer/probe sequences for carbapenemase/ESBL
RPA assays

| | | SEQ |
|---|---|---|
| | | ID |
| Name | Sequence (5′ to 3′) | NO |
| KPC_forward | CCCATCTCGGAAAAATATCTGACAACAGG | 1 |
| | C | |
| KPC_reverse | CTCCAGCTCCCAGCGGTCCAGACGGAACG | 2 |
| | TGGTAT | |
| KPC_probe | CGTGCAATACAGTGATAACGCCGCCGCCA | 3 |
| | A567GTTGCTGAAGGAGTT3 | |
| NDM_forward | GACCAGACCGCCCAGATCCTCAACTGGAT | 7 |
| | CAAGCA | |
| NDM_reverse | CTGGTTCGACAACGCATTGGCATAAGTCG | 8 |
| | CAA | |
| NDM_probe | CCCCGCCGCATGCAGCGCGTCCATACCGC | 9 |
| | CCA567TGTCCTGATGCGCG3 | |
| OXA-48- | CCTTATCGGCTGTGTTTTTGGTGGCATCG | 4 |
| like_forward | A | |
| OXA-48- | CCCGTTTAAGATTATTGGTAAATCCTTGCT | 5 |
| like_reverse | G | |
| OXA-48- | TGGCAAGAAAACAAAAGTTGGAATGCTCA | 6 |
| like_probe | C567ACTGAACATAAATCA3 | |
| VIM_forward | CCAGATTGCCGATGGTGTTTGGTCGCATA | 24 |
| | TCGC | |
| VIM_reverse | GGAAGTCCAATTTGCTTTTCAATCTCCGC | 25 |
| | GAGAAG | |
| VIM_probe_1 | CTCATTGTCCGTGATGGTGATGAGTTGCT | 26 |
| | 567GATTGATACAGCGTG3 | |
| VIM_probe_2 | CTTATCGTCCGCGATGCTGATGAGTTGCT | 27 |
| | 567TATTGATACAGCGTGG | |
| IMP_forward_1 | GGAATAGAGTGGCTTAATTCTCAATCTAT | 10 |
| IMP_forward_2 | GGAATAGAGTGGCTTAATTCTCGATCTAT | 11 |
| IMP_reverse | AAAAAACTTCAATTTTATTTTTAACTAGC | 12 |
| | C | |
| IMP_reverse_2 | AAAAAATTTCAATCTTTTTCTTAACTAAC | 13 |
| | C | |
| IMP_reverse_3 | AAAAAACTTCAATTTTGTTTTTAATTAGT | 14 |
| | G | |
| IMP_reverse_4 | AAAAAATTTCAATTTTATTTTTAAGTAGC | 15 |
| | C | |
| IMP_reverse_5 | AGAAAACTTCAATTTTGTTTTTGATAAGT | 16 |
| | G | |
| IMP_reverse_6 | AAAACACTTCTATTTTATCTTTCGCCAGC | 17 |
| | C | |
| IMP_reverse_7 | AAAAAACTTCAATTTTGTTTTTTATTAGT | 18 |
| | G | |
| IMP_reverse_8 | TTCAATTTTATTTTTAACTAGCCAATA | 19 |
| IMP_reverse_9 | TTCAATCTTTTTCTTAACTAACCAATA | 20 |
| IMP_reverse_10 | TTCAATTTTGTTTTTAATTAGTGAATA | 21 |
| IMP_reverse_11 | TTCTATTTTATCTTTCGCCAGCCAATA | 22 |
| IMP_probe | CCCACGTATGCATCTGAATTAACAAATG | 23 |
| | AAC56C7TAAAAAAGACGGTAA3 | |
| IMI_forward | CTTGAACGATATATCGGTGGTCCTGAGGG | 28 |
| IMI_reverse | TTCATCGCCAGGAATAGCTGTGTTTAGAT | 29 |
| | CT | |
| IMI_reverse_2 | GTGTCACGTTCATCGCCAGGAATAGCTGT | 30 |
| IMI_probe | CTAAATTCATGCGGTCGATTGGAGATAAA | 31 |
| | GAT567AGACTCGATCGTTG3 | |
| CTX-M- | CCGAATCTGTTAAATCAGCGAGTTGAGAT | 32 |
| 15_forward | CAAAAA | |
| CTX-M- | CTAGCCGGGCCGCCAACGTGAGCAATCAG | 34 |
| 15_reverse | CTTATT | |
| CTX-M- | GATTGCGGAAAAGCACGTCAATGGGACGA | 36 |
| 15_probe | 567CACTGGCTGAGCTTA3 | |
| CTX-M- | CACGTCAACGGCACAATGACGCTGGCAGA | 33 |
| 14_forward | ACTGAG | |
| CTX-M- | TCCAGACGAAACGTCTCATCGCCGATCGC | 35 |
| 14_reverse | GCGGGC | |
| CTX-M- | AGTACAGCGACAATACCGCCATGAACAAA | 37 |
| 14_probe | T56A7TGCCCAGCTCGGTGGC3 | |

where 5 = FamdT; 6 = abasic; 7 = bhqldT; 3 = propanol blocker

Primers and probes were synthesised using standard techniques. The primers and probes were prepared in 10 mM Tris 1 mM EDTA or water. Reactions were prepared using TwistAmp® Basic or Exo kits (TwistDx, Cambridge, UK) by adding template DNA, primers and probes (TwistAmp® Exo reactions only) to TwistAmp® reagents and initiating reactions with magnesium acetate.

TwistAmp® Basic reactions were incubated for 30 minutes at 39° C. and products purified using a Qiagen PCR purification kit (Qiagen, Germany) prior to visualisation on an agarose gel.

TwistAmp® Exo reactions were placed onto a real time PCR platform (ABI StepOne Plus) set to run for 30 minutes at 39° C. with fluorescence measurements taken every 60 seconds. Data was analysed using StepOne Plus software v2.3. Positive results were called when an exponential signal was detected and crossed a threshold (crossing threshold or Ct).

DNA purified using standard commercial kits from bacteria containing known positives or constructs containing individual carbapenemase/ESBL genes (Table 3) for each of the assays was used for initial assay evaluation. To establish the limit of detection (LoD) purified DNA was serially diluted 1:10 in 10 mM Tris, 1 mM EDTA or water (with the final dilution containing an estimated 1-10 genome copies) and tested against the assays.

TABLE 3
Bacterial strains used for evaluation of RPA assays

| Assay | Organism | Plasmid construct |
|---|---|---|
| KPC | Klebsiella pneumoniae NCTC | Escherichia coli + pACY184-KPC |
| | 13438 | |
| NDM | Klebsiella pneumoniae NCTC | Escherichia coli + pACY184-NDM |
| | 13443 | |
| OXA-48- | Klebsiella pneumoniae 16 | Escherichia coli + pACY184-OXA-48-like |
| like | | |
| VIM | Klebsiella pneumoniae NCTC | Escherichia coli + pACY184-VIM |
| | 13439 | |
| IMP | Escherichia coli NCTC 13476 | Escherichia coli + pACY184-IMP |
| IMI | Enterobacter asburiae ME52 | Escherichia coli + pACY184-IMI |
| CTX-M-15 | Escherichia coli NCTC 13441 | Escherichia coli + pACY184-CTX-M-15 |
| CTX-M-14 | Enterobacter cloacae NCTC 13464 | Not made |

To ensure all gene variants were detected for a particular assay, synthetic fragments representing sequence divergence across the primer and probe regions were obtained (IDT, Leuven, Belgium) and evaluated in the relevant assay. This was conducted for OXA-48-like VIM and IMP carbapenemase assays only since these show a greater degree of sequence diversity than the NDM or KPC genes A similar approach is also used for IMI to assess variance.

To evaluate the background fluorescence of primers/probes and determine the risk of cross contamination between reaction wells, no template control (NTC) reactions which contained no carbapenemase or ESBL target were included in every run.

To ensure that the LoD for target DNA within a particular assay is unaffected by the presence of a high background of non-target DNA, each assay was tested with purified target DNA at the lowest concentration detected in the presence of non-target bacterial DNA at a concentration of 1×10⁶ genome copies.

### Results

For all assays, the positives were detected as expected and no false amplification was observed in the NTC reactions. A limit of detection was established for all assays (Table 4) which was 100 genome copies or less. The time to positive, judged by the point at which the fluorescence signal crossed the threshold at the limit of detection, was recorded for each assay (Table 4). Times ranged from 10-16 minutes for 10-100 genome copies for the DNA evaluated. No effect was observed on the LoD for any of the assays when a high concentration of non-target DNA was included in the reaction.

TABLE 4
Limit of detection and time to detection

| | Limit of detection | Time to detection at |
|---|---|---|
| | (genome copies) | LoD (mins) |
| KPC | 100 | 14 |
| NDM | 10 | 16 |
| OXA-48- | 10 | 10 |
| like | | |
| VIM | 100 | 10 |
| IMP | 100 | 15 |
| IMI | 100 | 15 |
| CTX-M-15 | 10 | 13 |
| CTX-M-14 | 10 | 14 |

All gene variants were detected in the OXA-48 assay and all but one variant (VIM-7, which is highly divergent from other VIM variants) was detected in the VIM assay using VIM_probe_1. When VIM_probe_2 is used instead, this variant can be successfully detected. Using a combination of one of the IMP_forward and one of the IMP_reverse primers all of the IMP variants could be detected. Similarly, using a combination of one of the IMI_forward and one of the IMI_reverse primers all of the IMI variants tested could be detected

### Example 2: Further Validation of Primers and Probes

### Bacterial Isolates

### Test Isolates

The isolates (n=661) were comprised of 648 Enterobacteriaceae and 13 Pseudomonas spp. (see Table 5) that had been submitted to PHE's Antimicrobial Resistance and Healthcare Associated Infections (AMRHAI) reference unit for investigation of ‘unusual’ resistance, mainly to carbapenems.

The isolates had been identified using either API-20E tests (bioMerieux SA, Marcy-l'Etoile, France) or MALDI-ToF MS; Bruker Microflex LT (Brucker Daltonik, GmbH, Bremen, Germany). Antibiotic susceptibilities (minimum inhibitory concentrations—MICs) had been determined by BSAC agar dilutions and interpreted using BSAC breakpoints, where available. Carbapenemase genes had been identified within the AMRHAI reference unit using in-house PCR assays and/or a commercial microarray (Check-MDR CT102: Check-Points, Wageningen, The Netherlands).

The isolates included 611 carbapenemase positives and comprised 158 of KPC, 152 of NDM, 162 of OXA-48-like, 110 of VIM and 24 IMP producers. 5 isolates produced both NDM and OXA-48-like enzymes. A further 19 isolates demonstrated carbapenem resistance due to ESBL and/or AmpC activity plus porin loss and 31 isolates were carbapenem susceptible with ESBL's only. The latter two isolate groups represented predicted negatives for the carbapenemase assays.

TABLE 5
Isolates used as test panel

| | | Carbapenemase families | | | | | | |
|---|---|---|---|---|---|---|---|---|
| | | | | | OXA-48 | | | |
| | ESBL | | | OXA- | like + | | | |
| Species | CTX-M | KPC | NDM | 48 like | NDM | VIM | IMP | none |
| Klebsiella pneumoniae | 14 | 108 | 88 | 108 | 3 | 58 | 0 | 6 |
| Klebsiella oxytoca | 0 | 6 | 1 | 3 | 0 | 7 | 1 | 0 |
| Klebsiella sp. | 0 | 0 | 0 | 0 | 0 | 11 | 1 | 0 |
| Escherichia coli | 13 | 22 | 43 | 42 | 2 | 9 | 2 | 1 |
| Enterobacter spp. | 3 | 17 | 14 | 8 | 0 | 16 | 8 | 9 |
| Pseudomonas spp. | 0 | 0 | 0 | 0 | 0 | 0 | 11 | 2 |
| Others* | 1 | 5 | 6 | 1 | 0 | 9 | 1 | 1 |
| Total | 31 | 158 | 152 | 162 | 5 | 110 | 24 | 19 |

*24 isolates comprising Citrobacter spp. (n = 16), Raoultella spp. (n = 3), Leclercia adecarboxylata (n = 2), Serratia marcescens (n = 2) and Kluyvera georgina (n = 1)

### Negative Panel

The negative panel (Table 6) comprised of 27 bacterial and fungal strains not known to contain any carbapenemase or β-lactamase gene of interest for this set of assays. Human DNA was included to ensure no cross reaction with the assays. A further 19 organisms were included that contained the carbapenemase or ESBL genes of interest which would, depending on the assay being evaluated, would function as the positive control or evaluate whether other carbapenemase/ESBL genes cross-reacted i.e. generate false positive results.

TABLE 6
Negative test panel

| | | Resistance |
|---|---|---|
| Organism | Strain | genes |
| Candida albicans | NCPF 3179 | |
| Candida glabrata | NCPF 8018 | |
| Candida tropicalis | NCPF 8750 | |
| Acinetobacter | NCTC | |
| calcoaceticus | 7461 | |
| Acinetobacter iwoffi | NCTC | |
| | 7976 | |
| Bacillus subtilis | NCTC | |
| | 3610 | |
| Citrobacter freundii | NCTC | |
| | 9750 | |
| Enterobacter | NCTC | |
| cloacae | 13380 | |
| Enterococcus | NCTC | |
| faecalis | 12697 | |
| Enterococcus | NCIMB | |
| faecium | 2699 | |
| Escherichia coli | ATCC | |
| | 11303* | |
| Haemophilus | NCTC | |
| influenzae | 12699 | |
| Klebsiella | MGH | |
| pneumoniae | 78578 | |
| Moraxella | NCTC | |
| catarrhalis | 4103 | |
| Morganella | NCIMB | |
| morganii | 232 | |
| Proteus mirabilis | NCTC | |
| | 13376 | |
| Pseudomonas | PA01 | |
| aeruginosa | | |
| Salmonella | 140285 | |
| typhimurium | | |
| Salmonella | SL1344 | |
| typhimurium | | |
| Staphylococcus | NCTC | |
| aureus | 12493 | |
| Staphylococcus | ATCC | |
| epidermidis | 35984 | |
| Staphylococcus | NCTC | |
| saprophyticus | 7292 | |
| Streptococcus | NCTC | |
| agalactiae | 8181 | |
| Streptococcus mitis | NCIMB | |
| | 13770 | |
| Streptococcus | NCTC | |
| pneumoniae | 7465 | |
| Yersinia | NCTC | |
| enterocolitica | 10460 | |
| Yersinia | | |
| pseudotuberculosis | | |
| Human DNA* | | |
| Enterobacter | ME52 | IMI |
| asburiae | | |
| Enterobacter | NCTC | CTX-M-14 |
| cloacae | 13464 | |
| Escherichia coli | NCTC | CTX-M-15 |
| | 13441 | |
| Escherichia coli | NCTC | IMP |
| | 13476 | |
| Klebsiella | NCTC | NDM + |
| pneumoniae | 13443 | CTX-M-15 |
| Klebsiella | NCTC | KPC |
| pneumoniae | 13438 | |
| Klebsiella | NCTC | VIM-1 |
| pneumoniae | 13439 | |
| Pseudomonas | NCTC | VIM-10 |
| aeruginosa | 13437 | |
| Escherichia coli | TOP10 | pACYC184 + |
| | | CTX-M-15 |
| Escherichia coli | TOP10 | pACYC184 + |
| | | NDM |
| Escherichia coli | TOP10 | pACYC184 + |
| | | KPC |
| Escherichia coli | TOP10 | pACYC184 + |
| | | OXA-48 |
| Escherichia coli | TOP10 | pACYC184 + |
| | | VIM |
| Escherichia coli | TOP10 | pACYC184 + |
| | | IMI |
| Escherichia coli | TOP10 | pACYC184 + |
| | | IMP |
| Escherichia coli | TOP10 | pACYC184 + |
| | | empty |
| Escherichia coli | TOP10 | pACYC184 + |
| | | SHV-m109 |
| Escherichia coli | TOP10 | pACYC184 + |
| | | SHV-m43 |
| Escherichia coli | TOP10 | pACYC184 + |
| | | TEM |

*purchased as purified DNA

### Bacterial Culture and DNA Template Preparation

### Test Panel

Isolates were cultured on CLED agar plates, picked with a 14 loop and suspended in 2004 molecular grade water. The suspension was incubated at 95° C. for 10 minutes followed by centrifugation at 10 000×g for 3 minutes and 24 was used in the RPA reaction.

### Negative Panel

Bacterial or Candida strains were cultured on TSA agar, sub-cultured into TSB media and DNA extracted using standard commercially available extraction kits.

### Real Time Exo-RPA and Data Analysis

Template DNA and primers and probes for each assay (KPC, NDM, OXA-48, VIM, IMP, CTX-M-14 or CTX-M-15) were added to TwistAmp® Exo (TwistDx, Cambridge, UK) reagents and reactions initiated with the addition of magnesium acetate. Reactions were incubated for 30 minutes at 39° C. with fluorescence measurements being taken every 60 seconds. ABI StepOnePlus v2.3 software was used to analyse the data.

### Results

The correct carbapenemase gene was successfully detected for each assay in the 611 isolates evaluated that produced a KPC, NDM, VIM, OXA-48-like or IMP enzyme achieving 100% sensitivity for these targets (Table 7). Successful IMP detection was achieved using a number of assays combining one IMP_forward and one IMP_reverse primers from the selection described. The same approach is used for IMI detection. No amplification was observed for non-target genes or within the carbapenem resistant, carbapenemase negative or ESBL only panels. Additionally, no amplification was observed with the bacterial specificity panel and synthetic constructs containing non-target carbapenemase, ESBL or other β-lactamase genes with the assays therefore demonstrating 100% specificity. A small number of isolates were evaluated that contained more than one carbapenemase gene and these were all successfully detected by the assays.

TABLE 7
Results of assay

| | Number | CTX- | CTX- | | | | | |
|---|---|---|---|---|---|---|---|---|
| Panel | isolates | M-15 | M-14 | KPC | NDM | OXA | VIM | IMP |
| CRCN | 19 | 2 | 0 | 0 | 0 | 0 | 0 | 0 |
| CTX-M | 31 | 27 | 3 | 0 | 0 | 0 | 0 | 0 |
| KPC | 158 | 12 | 16 | 158 | 0 | 0 | 0 | 0 |
| NDM | 152 | 88 | 1 | 0 | 152 | 0 | 0 | 0 |
| OXA | 162 | 70 | 23 | 0 | 0 | 162 | 0 | 0 |
| NDM/OXA | 5 | 4 | 0 | 0 | 5 | 5 | 0 | 0 |
| VIM | 110 | 54 | 4 | 0 | 0 | 0 | 110 | 0 |
| IMP | 24 | Not | Not | 0 | 0 | 0 | 0 | 24 |
| | | tested | tested | | | | | |
| | | 257 | 45 | | | | | |

### Example 4: Work-Flow for Use of Assays at Point of Care to Inform Prescribing Practice

### Collection of Specimens and Sample Extraction.

Patient samples relevant to the clinical diagnosis and/or screening for the presence of carbapenemase-producing bacteria, are collected by any method known to those familiar with the art. Specific examples include, but are not limited to, the following samples and collection devices:

| Sample | Collection |
|---|---|
| Whole blood, serum, plasma | Vacutainer, syringe, blood-culture bottle |
| Urine | Mid-flow urine collection |
| | device; catheter, |
| | drainage bag |
| Sputum, bronchial | Universal, Bronchoscope, specialised |
| alveolar lavage (BAL), | catheter (miniBAL) |
| Tracheal aspirate | |
| Stools, faeces | Sterile sample collection |
| | device, rectal swab |
| Skin carriage | Swab |
| Wounds, abscesses | Drainage of pus into a sterile collection |
| | device, syringe, swab |

Similar sample collection systems may be applied for animal samples, either for primary diagnosis or screening for carbapenemase carriage, or in other forms of environmental sample (including sewage, drinking water samples, food etc.).

Concentration of the bacteria from the sample prior to analysis may be required depending on the number of bacteria in the sample. Concentration is achieved using a variety of techniques, including, but not limited to, filtration through suitable cut-off membranes, capture of bacteria on magnetic beads (on the basis of charge, general binding of bacteria cell wall components (e.g. vancomycin to conserved regions of the bacterial cell wall, molecules that bind to bacterial lipid A) or specific ligands specific to particular bacteria (antibodies, aptamers specific for particular surface ligands).

### Rapid Lysis of Sample and (Optional) Extraction of Bacterial Genomic DNA

Rapid lysis of bacteria, directly in appropriate clinical samples, is achieved by heating the sample to in excess of 85° C., preferably in excess of 95° C. for at least 5 minutes, preferably 15 minutes. Such direct processing of samples is useful where the amount of bacteria in a sample, is around 10000 to 100,000 colony forming units per ml (as it the case in many urinary tract infections). Under these conditions, the sensitivity of the assays developed in this application allows for direct detection without further purification or enrichment. This offers significant utility for point of care detection.

In clinical samples where the number of bacteria is lower, the total sample, or bacteria captured from it (as outlined above) is lysed and the bacterial genomic and plasmid DNA extracted. A variety of extraction protocols for enrichment of bacterial genomic DNA are commercially available and known to those familiar with the art.

Direct Amplification of Target Sequences from Lysed Clinical Sample

As described above, it may be possible to detect bacteria carrying one or more carbapenemase gene directly from certain sample types, such as urine samples which meet the clinical definition of a urinary tract infection, containing in excess of 10,000 colony forming units per ml.

In these conditions, heat lysis is achieved in a small volume of sample and the lysed sample mixed directly with an RPA masterix in separate wells containing primers and probes for each of the target carbapenemase genes or CTX-M. The assay is performed under the standard amplification conditions with real time detection of fluorescence. A small amount of reduction in signal (equivalent of up to 1-log reduction in titre) may be observed for some urine samples but this is deemed to be acceptable for this sample matrix.

Positive detection is confirmed when the fluorescence level exceeds the value of a no-template amplification control plus 3 standard deviations of the mean. Typically amplification is observed within 20 minutes and any amplification after this time point would usually be treated as a presumptive positive and a second test carried out.

### Interpretation of Assay Results as Basis for Prescribing Decisions

The assays developed have a very high sensitivity and specificity but the tests will only demonstrate the presence of one of the carbapenemase genes that is being tested for and/or the CTX-M gene. Other carbapenemase genes may be included in the test panel, but an entirely new carbapenemase would not be detected. Nor would bacteria where up-regulation of AmpC or perhaps other extended spectrum beta lactamases and porin loss may give a carbapenem-resistant phenotype.

Such carbapenem-resistance carbapenemase-negative bacteria remain uncommon and as such this does not significantly compromise the performance and utility of the assay. They do impact on treatment decisions that may be made on the basis of the test results derived from assays of the invention.

Although local decisions may differ, a clinician may choose to use the carbapenemase gene detection panel as the basis for not prescribing carbapenem antibiotics, such as meropenem and imipenem, which would not be likely to be effective against an infection which test positive for one or more of these genes. Such a “rule-out” test has significant clinical value in directing precious antibiotic, only to infections which are likely to respond, in priming the clinician to look for other treatments which are more likely to be effective and in the cost saved for not using ineffective drugs.

### Re-Test Protocol (Optional)

If the event of a late RPA amplification or if additional certainty is required, a second sample from the patient may be tested to confirm the presence or absence of carbapenemase gene-carrying bacteria in a sample. The second sample will be taken independently of the first and this also reduces the potential for contamination. An amplification for one of the gene targets and/or a second late amplification would likely be interpreted as a genuine detection.

### Example 5: Use of Test as Rapid Confirmatory Test for Detection of Presumptive Carbapenemase Producing Bacteria on Culture Plates

Aside from point of care diagnosis, the rapid output of the carbapenemase gene detection panel of the invention has utility in allowing an early assessment of the probable susceptibility of the bacteria from an infection.

Bacteria from a clinical sample, referred to a hospital microbiology department are typically grown on agar plates for 16-24 hours on receipt. Single colonies or a loop-full of the primary streak, representative of the bacteria in a sample, may be picked from the agar plates and resuspended in a simple salt buffer and heat lysed as described above (85-95° C. for 5-15 minutes, followed optionally by centrifugation at 13000 g for 10 minutes). The lysate is mixed directly with mastermixes for each of the genes to be detected and a standard amplification carried out. Positive amplification is determined as described above.

This rapid confirmatory test allows a positive result, likely to represent the presence of a bacterial strain which will not respond to carbapenem treatment, to be communicated to the clinician. This provides a considerable time saving (in some cases >48 hours) compared to other susceptibility test protocols.

### Example 6: Use of Test as Rapid Confirmatory Test for Detection of Carbapenemase Producing Bacteria in Blood Culture Specimen

Blood culture remains the primary means of diagnosing potential bacteremia or sepsis. Whilst time to treat is a critical parameter, a test which would allow an informed decision to be made on use of carbapenems would have significant clinical value as defined in Example 4. The speed and sensitivity of the current test might be usefully employed in allowing early analysis of growth in blood bottles used to diagnose sepsis, again allowing an early indication of likely treatment efficacy. Such a test would use the following or similar protocol.

Blood samples are collected using standard protocols and cultured as per manufacturer's instructions. At 8 hours post-culture, the presence of growth is identified by turbidity, the production of carbon dioxide, changes in an indicator dye or other means known to those familiar with the art.

A sample from the bottle is taken using sterile means and the sample processed to extract bacterial genomic and plasmid DNA. A wide variety of methods are useful for such extraction purposes, including bead based DNA capture methods.

The extracted DNA is mixed with mastermixes for each of the carbapenemase genes and a standard amplification carried out; positive amplification being determined by real-time generation of fluorescence signal as described above.

The results from such a test are used as a rule out for continued treatment with carbapenems. The results from such a test would be available 24-72 hours earlier than those from traditional susceptibility test methods and as such this has significant value for the clinician.

### Example 7: Use of Combined Carbapenemase Assay Panel to Inform Prescribing Decisions

The clinical value of the assays developed is in providing a rule out assay to inform prescribing practice. To achieve this aim, the assays described herein are used in combination, to provide an overall assessment of the presence of carbapenemase genes likely to result in treatment failure. An example of the use of such a panel assay is described below.

DNA is extracted from bacteria within a clinical sample, colony isolated by culture or positive blood-culture. The DNA is mixed with specific primers and probes for the carbapenemase genes of interest. The choice of genes may be dictated by local prevalence estimates, or may include all of the carbapenemase genes identified here. It is likely that the subset would include a minimum set of primers and probes for KPC, NDM, Oxa-48, IMP, IMI and VIM. Optionally, the primer sets for CTX-M14 and M15 might be included. The primers and probes could be combined into a single multiplex assay, two or more smaller multiplex assays (e.g. 2 reactions each with 3 primer/probe sets; such as reaction 1 KPC, NDM, Oxa-48 and reaction 2 IMP, IMI, VIM) or perhaps most preferentially carried out as single assays on a microfluidic device. The detection method could be achieved by real-time PCR or, more preferably using RPA. A positive amplification control would also be included to ensure that the assay results were valid.

The output of the assay would be a single positive or negative result for the presence of one or more of the carbapenemase genes. A carbapenemase-positive assay result would mean that a clinician could not safely treat with carbapenem antibiotics (e.g. meropenem, imipenem) and should select an alternative treatment option. A carbapenemase-negative assay result would suggest that there is a much greater chance of a treatment with a carbapenem being effective and would encourage the use of this antibiotic if other symptoms and clinical setting were appropriate. The latter result cannot rule out the possibility of resistance to carbapenems due to either a novel carbapenemase gene or resistance mediated by other mechanisms.

1-27. (canceled)

28. A method for determining presence and/or amount of one or more carbapenemase-producing bacteria in a sample comprising determining presence and/or amount of a KPC carbapenemase gene, an OXA-48-like carbapenemase gene, and a VIM carbapenemase gene in said sample, wherein:
(a) the presence and/or amount of the KPC gene is determined using:
i. a forward primer comprising the nucleic acid sequence of SEQ ID NO: 1, or a nucleic acid sequence having at least 90% sequence identity to the full-length of the nucleic acid sequence of SEQ ID NO: 1;
ii. a reverse primer comprising the nucleic acid sequence of SEQ ID NO: 2, or a nucleic acid sequence having at least 90% sequence identity to the full-length of the nucleic acid sequence of SEQ ID NO: 2; and
iii. a probe comprising the nucleic acid sequence of SEQ ID NO: 3, or a nucleic acid sequence having at least 90% sequence identity to the full-length of the nucleic acid sequence of SEQ ID NO: 3;
(b) the presence and/or amount of the OXA-48-like gene is determined using:
i. a forward primer comprising the nucleic acid sequence of SEQ ID NO: 4, or a nucleic acid sequence having at least 90% sequence identity to the full-length of the nucleic acid sequence of SEQ ID NO: 4;
ii. a reverse primer comprising the nucleic acid sequence of SEQ ID NO: 5, or a nucleic acid sequence having at least 90% sequence identity to the full-length of the nucleic acid sequence of SEQ ID NO: 5; and
iii. a probe comprising the nucleic acid sequence of SEQ ID NO: 6, or a nucleic acid sequence having at least 90% sequence identity to the full-length of the nucleic acid sequence of SEQ ID NO: 6; and
(c) the presence and/or amount of the VIM gene is determined using:
i. a forward primer comprising the nucleic acid sequence of SEQ ID NO: 24, or a nucleic acid sequence having at least 90% sequence identity to the full-length of the nucleic acid sequence of SEQ ID NO: 24;
ii. a reverse primer comprising the nucleic acid sequence of SEQ ID NO: 25, or a nucleic acid sequence having at least 95% sequence identity to the full-length of the nucleic acid sequence of SEQ ID NO: 25; and
iii. a probe comprising the nucleic acid sequence of SEQ ID NO: 26 or 27, or a nucleic acid sequence having at least 90% sequence identity to the full-length of the nucleic acid sequence of SEQ ID NO: 26 or 27.

29. The method of claim 28, wherein the presence and/or amount of:
(a) a KPC gene, an OXA-48-like gene, a VIM gene and an NDM gene;
(b) a KPC gene, an OXA-48-like gene, a VIM gene, an NDM gene and an IMI gene;
(c) a KPC gene, an OXA-48-like gene, an NDM gene, an IMP gene and a VIM gene; or
(d) a KPC gene, an OXA-48-like gene, an NDM gene, an IMP gene, a VIM gene and an IMI gene;
is determined.

30. The method of claim 29, wherein the presence and/or amount of the NDM gene is determined using at least one oligonucleotide sequence specific for the NDM gene, which at least one oligonucleotide comprises a nucleic acid sequence selected from:
(a) a nucleic acid sequence of SEQ ID NO: 7, or a nucleic acid sequence having at least 90% sequence identity to the full-length of the nucleic acid sequence of SEQ ID NO: 7;
(b) a nucleic acid sequence of SEQ ID NO: 8, or a nucleic acid sequence having at least 90% sequence identity to the full-length of the nucleic acid sequence of SEQ ID NO: 8; and/or
(c) a nucleic acid sequence of SEQ ID NO: 9, or a nucleic acid sequence having at least 90% sequence identity to the full-length of the nucleic acid sequence of SEQ ID NO: 9;
wherein the at least one oligonucleotide of (a) is a forward primer, the at least one oligonucleotide of (b) is a reverse primer; and/or the at least one oligonucleotide of (c) is a probe.

31. The method of claim 29, wherein the presence and/or amount of the IMP gene is determined using at least one oligonucleotide sequence specific for the IMP gene, which at least one oligonucleotide comprises a nucleic acid sequence selected from:
(d) a nucleic acid sequence of SEQ ID NO: 10 or 11, or a nucleic acid sequence having at least 90% sequence identity to the full-length of the nucleic acid sequence of SEQ ID NO: 10 or 11;
(e) a nucleic acid sequence of any one of SEQ ID Nos: 12-22, or a nucleic acid sequence having at least 90% sequence identity to the full-length of the nucleic acid sequence of any one of SEQ ID Nos: 12-22; and/or
(f) a nucleic acid sequence of SEQ ID NO: 23, or a nucleic acid sequence having at least 90% sequence identity to the full-length of the nucleic acid sequence of SEQ ID NO: 23;
wherein the at least one oligonucleotide of (a) is a forward primer, the at least one oligonucleotide of (b) is a reverse primer; and/or the at least one oligonucleotide of (c) is a probe.

32. The method of claim 29, wherein the presence and/or amount of the IMI gene is determined using at least one oligonucleotide sequence specific for the IMI gene, which at least one oligonucleotide comprises a nucleic acid sequence selected from:
(a) a nucleic acid sequence of SEQ ID NO: 28, or a nucleic acid sequence having at least 90% sequence identity to the full-length of the nucleic acid sequence of SEQ ID NO: 28;
(b) a nucleic acid sequence of SEQ ID NO: 29 or 30, or a nucleic acid sequence having at least 90% sequence identity to the full-length of the nucleic acid sequence of SEQ ID NO: 29 or 30; and/or
(c) a nucleic acid sequence of SEQ ID NO: 31, or a nucleic acid sequence having at least 90% sequence identity to the full-length of the nucleic acid sequence of SEQ ID NO: 31;
wherein the at least one oligonucleotide of (a) is a forward primer, the at least one oligonucleotide of (b) is a reverse primer; and/or the at least one oligonucleotide of (c) is a probe.

33. The method of claim 29, wherein the presence and/or amount of the NDM gene, IMP gene, and/or IMI gene is determined using:
(a) the corresponding forward primer sequences of claim 3;
(b) the corresponding reverse primer sequences of claim 3;
(c) the corresponding forward and reverse primer sequences of claim 3;
and/or
(d) the corresponding forward primer, reverse primer and probe sequences of claim 3.

34. The method of claim 28, further comprising determining:
(a) the presence and/or amount of one or more additional carbapenemase gene; and/or
(b) the presence and/or amount of one or more CTX-M β-lactamase.

35. The method of claim 34, wherein the one or more additional carbapenemase gene is selected from an SME gene, a GES gene and an SPM gene.

36. The method of claim 34, wherein the presence and/or amount of the one or more additional carbapenemase gene is determined using at least one oligonucleotide specific for said one or more additional carbapenemase gene, and/or the presence and/or amount of the one or more CTX-M β-lactamase is determined using at least one oligonucleotide specific for CTX-M β-lactamase.

37. The method of claim 36, wherein the at least one oligonucleotide specific for CTX-M β-lactamase is selected from:
(a) a nucleic acid sequence of SEQ ID NO: 32 or 33, or a nucleic acid sequence having at least 90% sequence identity to the full-length of the nucleic acid sequence of SEQ ID NO: 32 or 33;
(b) a nucleic acid sequence of SEQ ID NO: 34 or 35, or a nucleic acid sequence having at least 90% sequence identity to the full-length of the nucleic acid sequence of SEQ ID NO: 34 or 35; and/or
(c) a nucleic acid sequence of SEQ ID NO: 36 or 37, or a nucleic acid sequence having at least 90% sequence identity to the full-length of the nucleic acid sequence of SEQ ID NO: 36 or 37;
wherein the at least one oligonucleotide of (a) is a forward primer, the at least one oligonucleotide of (b) is a reverse primer; and/or the at least one oligonucleotide of (c) is a probe.

38. The method of claim 28, wherein the presence and/or amount of the KPC gene, the OXA-48-like gene, the NDM gene, the IMP gene, the VIM gene, the IMI gene and/or the one or more additional carbapenemase gene and/or the one or more CTX-M β-lactamase is determined by PCR and/or hybridisation.

39. The method of claim 38, wherein the presence and/or amount of the KPC gene, the OXA-48-like gene, the NDM gene, the IMP gene, the VIM gene, the IMI gene and/or the one or more additional carbapenemase gene and/or the one or more CTX-M β-lactamase is determined by Recombinase Polymerase Amplification (RPA) or real-time PCR.

40. The method of claim 28, wherein:
(a) the presence and/or amount the KPC gene is compared with the presence and/or amount of the KPC gene in a control sample;
(b) the presence and/or amount the OXA-48-like gene is compared with the presence and/or amount of the OXA-48-like in a control sample;
(c) the presence and/or amount the NDM gene is compared with the presence and/or amount of the NDM gene in a control sample;
(d) the presence and/or amount the IMP gene is compared with the presence and/or amount of the IMP gene in a control sample;
(e) the presence and/or amount the VIM gene is compared with the presence and/or amount of the VIM gene in a control sample;
(f) the presence and/or amount the IMI gene is compared with the presence and/or amount of the IMI gene in a control sample;
(g) the presence and/or amount the one or more additional carbapenemase gene is compared with the presence and/or amount of the one or more additional carbapenemase gene in a control sample; and/or
(h) the presence and/or amount the one or more CTX-M β-lactamase is compared with the presence and/or amount of the one or more CTX-M β-lactamase in a control sample.

41. A method for diagnosing a carbapenem-resistant bacteria infection in an individual comprising carrying out a method according to claim 28 on a sample obtained from the individual.

42. The method of according to claim 28, wherein the sample is a sample of whole blood, serum, plasma, cerebral spinal fluid, saliva, urine, cells, a cellular extract, a stool sample, a tissue sample or a tissue biopsy, or a swab from an individual, such as a rectal swab, or a swab from the environment.

43. A device for use in the method of according to claim 28, which comprises:
(a) a forward primer, a reverse primer and a probe specific for each of the KPC gene, the OXA-48-like gene, and the VIM gene, wherein the forward primer, reverse primer and probe for each of the KPC gene, the OXA-48-like gene, and the VIM gene, are the forward primer, reverse primer and probe as defined in claim 28; and
(b) one or more oligonucleotide specific for the NDM gene, the IMP gene, the IMI gene and/or the one or more additional carbapenemase gene, wherein the one or more oligonucleotide specific for the NDM gene, the IMP gene, the IMI gene and/or the one or more additional carbapenemase gene is an oligonucleotide.

44. The device of claim 43, wherein the one or more oligonucleotide is immobilized on a surface.
